(19) United States
(12) Patent Application Publication
(10) Pub. No.: US 2012/0077211 A1
(43) Pub. Date: Mar. 29, 2012

(54) STABLE COMPOSITIONS COMPRISING CHROMOGENIC COMPOUNDS AND METHODS OF USE

(75) Inventors: Lawrence Greenfield, Eugne, OR (US); Shawn Starkenburg, Los Alamos, NM (US); Matthew Shallice, Eugene, OR (US); Julie Nyhus, Terrace, WA (US); Louis Leong, Junction City, OR (US)

(73) Assignee: LIFE TECHNOLOGIES CORPORATION, Carlsbad, CA (US)

(21) Appl. No.: 13/139,873

(22) PCT Filed: Dec. 15, 2009
(86) PCT No.: PCT/US09/68067

§ 371 Date: Dec. 19, 2011

Related U.S. Application Data
(60) Provisional application No. 61/122,692, filed on Dec. 15, 2008.

(51) Int. Cl. G01N 33/53; C12Q 1/28; C09K 3/00; C07C 211/54

(52) U.S. Cl. 435/7.21; 564/7; 564/5; 435/28; 252/182.29

(57) ABSTRACT

Compositions, assays, methods, and kits are disclosed for use in applications that utilize oxidation of a chromogenic electron donor such as diaminobenzidine (DAB) to generate a signal. Applications include, but are not limited to, immunohistochemistry, chromogenic in situ hybridization, Western blots, Northern blots, Southern blots, ELISA assays, and microarray detection. The compositions, assays, methods, and kits disclosed herein make use of a novel, stabilized formulation of DAB and a novel, stabilized formulation of hydrogen peroxide.

### RELATED APPLICATIONS

This application is a U.S. National Stage Application of PCT application no. PCT/US2009/068067, filed Dec. 15, 2009, which claims priority to U.S. application No. 61/122,692, filed Dec. 15, 2008, which disclosures are herein incorporated by reference in their entirety.

### FIELD OF THE INVENTION

The present invention relates to the field of detection of biological targets in general, and nucleic acid and protein targets in particular, where oxidation of a chromogenic electron donor is utilized to generate a detectable signal.

### BACKGROUND OF THE INVENTION

Approximately 75% of the $1.4B histology market resides in the United States. The secondary staining segment (tissue analysis) is currently $600M and is expected to reach $1B by 2011, with 12% to 15% growth per annum. Key products within the histology area include immunohistochemistry (IHC) antibodies and detection reagents, H & E stains (for primary staining), special stains (for infectious disease), chromogenic in situ hybridization (CISH) reagents (e.g., DNA/RNA probes), automation systems (for tissue prep/staining) and imaging systems. Key customer drivers include quality and availability of stains and reagents, automation capabilities, breadth of menu (including antibodies, probes, and detection systems) and pricing.

Successful IHC, CISH, ELISA, and like assays depend on sensitive detection reagents with minimal background signals. Detection systems (e.g., kits) based on oxidation of a chromogenic electron donor such as 3,3′-diaminobenzidine (referred to herein as DAB) can be associated with weak signals, or no signals at all, as well as the presence of a significant amount of DAB background, i.e., the DAB chromogen reagent may undergo unwanted premature oxidation, turn dark brown and, on occasion, form precipitates. Accordingly, some CISH reagents, for example, which result from mixing DAB, hydrogen peroxide and an aqueous buffer, require use within one hour of being mixed.

In order to produce a chromogenic electron donor-based detection system that would be easy to manufacture, contain stable components, improve the resultant signal intensity and simplify the immunohistochemistry staining protocol to minimize user error(s), development efforts were focused on developing a two-component system including a stabilized formulation of DAB, as an exemplary chromogenic electron donor, and a stabilized formulation of hydrogen peroxide, as an exemplary peroxide.

Hydrogen peroxide decomposes to water and oxygen and requires stabilization when stored for prolonged periods of time. Patented compositions and methods for stabilization of hydrogen peroxide include those described in U.S. Pat. Nos. 3,811,833; 3,933,982; 4,070,442; 4,132,762; 4,133,869; 4,304,762; 4,770,808; 4,915,781; 4,981,662; 5,155,025; 5,804,404; and 6,677,466, the disclosures of which are hereby incorporated herein by reference. Classic hydrogen peroxide stabilizing agents described in the literature include: phosphoric acid; tin oxides, such as sodium stannate; dipicolinic acid; sodium pyrophosphate or organic phosphonic acids or their salts; acetone; 8-hydroxyquinoline; sulfolenes; sulfolanes; sulfoxides; sulfones; dialkylaminothiomethyl groups; thioalkylsulfonic acids; aliphatic amines; benzotriazole; nitro-substituted organic compounds, such as nitrobenzene sulfonic acids; thiosulfate; organic compounds, such as organic chelating agents or organic acids; ethylenediamine tetraacetic acid (EDTA); and amino tri-(lower alkylidene phosphonic acid). Most of the prior art compounds and compositions show some stabilization of hydrogen peroxide under acidic conditions, but have poor stabilizing effect under alkaline conditions.

In contrast to the vast amount of stabilizing agents described for hydrogen peroxide, there is little literature describing compositions or methods for stabilizing a chromogenic electron donor such as DAB. Temporary stabilization of DAB has typically been achieved by formulating under acidic conditions, where all four aromatic amino groups of DAB are protonated. However, despite the acidification, DAB continues to oxidize upon storage. Accordingly, a truly stabilized formulation of DAB has heretofore not been realized.

### SUMMARY OF THE INVENTION

The present invention provides a novel, stabilized formulation of DAB, which formulation includes a chelating agent, an antioxidant, and an organic polyol the combination of which reduces unwanted oxidation and/or precipitation of DAB in aqueous solution.

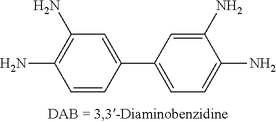

The present invention further provides a novel, stabilized formulation of hydrogen peroxide, which formulation includes a buffer, a chelating agent, and a nitrogen-containing organic compound the combination of which reduces the rate of hydrogen peroxide decomposition in aqueous solution. Upon combination of the aforementioned stabilized formulations, the present invention also provides a horse radish peroxidase (HRP) reaction buffer wherein premature oxidation and/or unwanted precipitation of DAB in the absence of added HRP is reduced. Furthermore, combination of the aforementioned stabilized formulations essentially eliminates any requirement for immediate use of the HRP reaction buffer, thereby lending the stabilized formulations themselves and combinations thereof to use in automation.

Additional aspects, features and advantages of the present invention will become apparent from the following detailed description. It should be understood, however, that the detailed description and the specific examples that follow, while indicating preferred embodiments of the present invention, are given by way of illustration only. It is expected that various changes and modifications within the spirit and scope of the present invention will become apparent to those skilled in the art

### BRIEF DESCRIPTION OF THE FIGURES

FIG. 1 shows stability of DAB formulations: DAB was formulated in a variety of buffers and incubated at 37° C. The 0 Hr, 11.8 Hr (top) and 110.8 Hr (bottom) values listed in Table 12 are graphically shown to highlight lead formulation candidates.

FIG. 2 shows stability of DAB formulations: DAB was formulated in a variety of buffers and incubated at 37° C. Representative data for stability in water, 1 mM DTPA and 85% methanol are plotted from Table 12. A regression line was drawn through the data to demonstrate the linearity of the data and the slope of the line determined.

FIG. 3 shows stability of DAB formulations at 37° C.: Graphical representation of the data listed in Table 13.

FIG. 4 shows stability of DAB Formulations: Graphical representation of the 158-hour time point listed in Table 15. DAB was formulated with different additives and incubated at 37° C. Prior to incubation and at 158 hours, the absorbance at 520 nm was measured using a Nanodrop ND-1000 spectrometer. The absorbance at each time point and the ratio of the absorbance at 158 hours and prior to incubation are plotted.

FIG. 5 shows an assay for HRP, hydrogen peroxide and DAB based on the assay of Herzog, V. and Fahimi, H D. (A New Sensitive Colorimetric Assay for Peroxidase Using 3,3′-Diaminobenzidine as Hydrogen Donor. Analytical Biochemistry 55: 554-562 (1973)). Assay was performed in 100 mM Citrate, pH 5, 0.1% gelatin, 0.5 mM DAB and the indicated final concentration of hydrogen peroxide. The reaction was initiated by the addition of 19 ng of HRP, the reaction mixture was incubated at ambient temperature and the absorbance at 465 nm or 466 nm was measured every 20 seconds using the NanoDrop ND-1000 spectrometer. The lines indicate a linear regression drawn through the initial time points.

FIG. 6 shows an HRP assay: The effect of hydrogen peroxide concentration on the initial reaction rate was determined from the data shown in Table 15. Assay was performed in 100 mM Citrate, pH 5, 0.1% gelatin, 0.5 mM DAB (top graph) or 2.5 mM DAB (middle and bottom graph) at the indicated final concentration of hydrogen peroxide. The reaction was initiated by the addition of 18.7 ng of HRP (top and middle graph) or 9.3 ng of HRP (bottom graph), the reaction mixture was incubated at ambient temperature and the absorbance at 465 nm or 466 nm was measured every 20 seconds using the NanoDrop ND-1000 spectrometer. The data represent a plot of the slope of the linear regression drawn through the initial time points for each hydrogen peroxide concentration.

FIG. 7 shows the stability of hydrogen peroxide when formulated in 200 mM Sodium Citrate, pH 5.0, 1 mM DTPA, and 50 mM Imidazole. The formulation of 200 mM Sodium Citrate, pH 5.0, 1 mM DTPA, 50 mM imidazole, and 0.03% Hydrogen peroxide was incubated at 37° C. for the indicated times. Hydrogen peroxide was measured indirectly by measuring the HRP activity following the addition of 46.6 ng HRP (93.3 ng/mL) and DAB.

FIG. 8 shows the effect of DAB concentration and presence of imidazole on HRP activity. 18.7 ng HRP (37 ng/mL) was assayed in 200 mM Sodium Acetate, pH 5, 1.5 mM DAB, 0.015% H₂O₂, 0.2% Gelatin with the indicated additives. The values are indicated in Table 21.

FIG. 9 shows the stability of DAB and Hydrogen Peroxide buffer formulations. CISH was performed using Invitrogen SuperPicture™ kit DAB, buffer and hydrogen peroxide (C1) or 200 mM Sodium Acetate, pH 5.0, 1 mM DTPA, 50 mM Imidazole, 0.03% Hydrogen Peroxide that had been stored for 27 days at 37° C. and 50 mM DAB, 10 mM DTPA, 65% Propylene Glycol, 10 mM Sodium Sulfite that had been stored at 37° C. for 2 days (C2=test reagents). Note the stronger CISH signal with the test reagents.

FIG. 10 shows a close examination of PowerVision™ Reagents with HRP Activity. HRP (9.3 μg/mL) was assayed in 0.2% gelatin in the indicated buffers. Absorbance at 465 nm was monitored and recorded. Note that the key component to PowerVision™ Reagents appears to be their “DAB.”

FIG. 11 shows the effect of buffer and DAB source on HRP activity.

FIG. 12 shows the effect of buffer on ImmPACT™ DAB performance.

FIG. 13 shows a comparison of competitive DAB detection reagents. 46.65 ng HRP (46.65 ng/mL) was assayed using the indicated DAB detection systems. The “New Detection Reagents” consists of 200 mM Sodium Acetate, pH 5.0, 1 mM DTPA, 50 mM Imidazole, 0.03% Hydrogen Peroxide and 50 mM DAB, 10 mM DTPA, 65% Propylene Glycol, 10 mM Sodium Sulfite. Note the superior performance of the New Detection Reagents compared to the Invitrogen SuperPicture™ kit formulation.

FIG. 14 shows Next Generation DAB Stability. In Panels A and B, DAB from the Invitrogen-Zymed SuperPicture™ kit (87-9633) and Vision ImmPact™ DAB (SK-4105) were stored at 4° C. and freshly prepared according to the manufacturer's instructions. In Panel C, a prototype lot of Next Generation DAB was freshly formulated on the day of the experiment. In Panel D, a separate prototype lot of the Next Generation DAB formulation was incubated at 37° C. for 9 days and monitored for performance via IHC.

### DETAILED DESCRIPTION OF THE INVENTION

Before describing the present invention in detail, it is to be understood that this invention is not limited to specific compositions or process steps per se, as such may vary. Further, it should be noted that, as used in this specification and the appended claims, the singular form “a”, “an” and “the” include plural references unless the context clearly dictates otherwise. Unless defined otherwise, all technical and scientific terms used herein have the same meaning as commonly understood by one of ordinary skill in the art to which this invention is related.

As used herein, the term “Next Generation Detection Reagents” refers to the final paired formulations (developed herein) of the DAB chromogen, i.e., 200 mM DAB, 20 mM HCl, 10 mM DTPA, 1 mM Sodium Sulfite, 65% Propylene Glycol, and the Hydrogen Peroxide Buffer, i.e., 200 mM Sodium Acetate, pH 5.5, 50 mM Imidazole, 1 mM DTPA, and 0.03% Hydrogen Peroxide. “Next Generation DAB chromogen” refers to the final formulation of the DAB chromogen, i.e., 200 mM DAB, 20 mM HCl, 10 mM DTPA, 1 mM Sodium Sulfite, and 65% Propylene Glycol. “Next Generation Hydrogen Peroxide Buffer” refers to the final formulation of the Hydrogen Peroxide Buffer, i.e., 200 mM Sodium Acetate, pH 5.5, 50 mM Imidazole, 1 mM DTPA, and 0.03% Hydrogen Peroxide. “Next Generation” products as described herein are those that are based on “Next Generation Detection Reagents.”

### Illustrative Embodiments of the Invention

The present invention provides compositions, assays, methods, and kits for use in applications that utilize oxidation of a chromogenic electron donor such as diaminobenzidine (DAB) to generate a signal. Applications include, but are not limited to, immunohistochemistry (IHC), chromogenic in situ hybridization (CISH), Western blots, Northern blots, Southern blots, ELISA assays, and microarray detection. The compositions, assays, methods, and kits of the present invention make use of a novel, stabilized formulation of DAB and a novel, stabilized formulation of hydrogen peroxide.

In one illustrative embodiment, the present invention provides a novel, stabilized formulation of DAB, which formulation includes a chelating agent, an antioxidant, and an organic polyol the combination of which reduces unwanted oxidation and/or precipitation of DAB in aqueous solution. In one illustrative aspect, chelating agents such as DTPA or EDTA, for example, may be used in the formulation. In another illustrative aspect, antioxidants such as sodium sulfite or sodium metabisulfite, for example, may be used in the formulation. In yet another illustrative aspect, organic polyols such as, for example, propylene glycol or a sugar (e.g., ribose) may be used in the formulation.

In another illustrative embodiment, the present invention provides a novel, stabilized formulation of hydrogen peroxide, which formulation includes a buffer, a chelating agent, and a nitrogen-containing organic compound the combination of which reduces the rate of hydrogen peroxide decomposition in aqueous solution. In one illustrative aspect, a buffer such as sodium acetate may be used in the formulation. In another illustrative aspect, chelating agents such as DTPA or EDTA, for example, may be used in the formulation. In yet another illustrative aspect, nitrogen-containing compounds such as imidazole, for example, may be used in the formulation.

Upon combination of the aforementioned stabilized formulations in another illustrative embodiment, the present invention also provides a horse radish peroxidase (HRP) reaction buffer wherein premature oxidation and/or unwanted precipitation of DAB in the absence of added HRP is reduced. In one illustrative aspect, combination of the aforementioned stabilized formulations essentially eliminates any requirement for immediate use of the resulting HRP reaction buffer.

In another illustrative embodiment, the present invention provides assays and methods for use in applications that utilize oxidation of a chromogenic electron donor such as diaminobenzidine (DAB) to generate a signal (see, paragraph [0026] for several exemplary applications). Assays and methods provided by the present invention make use of a novel, stabilized formulation of DAB and a novel, stabilized formulation of hydrogen peroxide. In one illustrative aspect, a novel, stabilized formulation of DAB (for use in assays and methods of the present invention) includes a chelating agent, e.g., DTPA, an antioxidant, e.g., sodium sulfite, and an organic polyol, e.g., propylene glycol, the combination of which reduces unwanted oxidation and/or precipitation of DAB in aqueous solution. In another illustrative aspect, a novel, stabilized formulation of hydrogen peroxide (for use in assays and methods of the present invention) includes a buffer, e.g., sodium acetate, a chelating agent, e.g., DTPA, and a nitrogen-containing organic compound, e.g., imidazole, the combination of which reduces the rate of hydrogen peroxide decomposition in aqueous solution.

In another illustrative embodiment, the present invention provides kits for use in detection applications that utilize oxidation of a chromogenic electron donor such as diaminobenzidine (DAB) to generate a signal (see, paragraph [0026] for several exemplary applications). Kits provided by the present invention for use in detection applications make use of a novel, stabilized formulation of DAB and a novel, stabilized formulation of hydrogen peroxide. In one illustrative aspect, a novel, stabilized formulation of DAB (for use in kits of the present invention) includes a chelating agent, e.g., DTPA, an antioxidant, e.g., sodium sulfite, and an organic polyol, e.g., propylene glycol, the combination of which reduces unwanted oxidation and/or precipitation of DAB in aqueous solution. In another illustrative aspect, a novel, stabilized formulation of hydrogen peroxide (for use in kits of the present invention) includes a buffer, e.g., sodium acetate, a chelating agent, e.g., DTPA, and a nitrogen-containing organic compound, e.g., imidazole, the combination of which reduces the rate of hydrogen peroxide decomposition in aqueous solution.

### Particular Aspects of the Invention:

In order to produce a chromogenic electron donor-based detection system that would be easy to manufacture, contain stable components, improve the resultant signal intensity and simplify the immunohistochemistry staining protocol to minimize user error(s), it was first necessary to understand any limitations associated with a typical DAB-based detection kit.

Detection kits for anatomical pathology, for example those available from Invitrogen, have typically consisted of three components: Reagent B1 or I1, i.e., the buffer/substrate buffer component; Reagent B2 or I2, i.e., the DAB chromogen; and Reagent B3 or I3, i.e., hydrogen peroxide. Color development is performed by mixing each of the three components with 1 mL of water, which is supplied by the user. For in situ hybridization kits, one drop of each of the three reagents is added to 1 mL of water. However, for immunohistochemistry kits such as the Invitrogen SuperPicture™ kit and the Histostain® kit, two drops of the DAB chromogen are added with one drop of the buffer and hydrogen peroxide to 1 mL of water. The DAB chromogen is formulated in 85% methanol. Because of the inherent surface tension characteristics of methanol, the size of a DAB chromogen-containing drop can vary over a wide range of volumes.

### Troubleshooting the SuperPicture™ Kit

The primary limitation of the SuperPicture™ kit was identified to be the Buffer/Substrate Buffer (Reagent B1 or I1) having insufficient buffering capacity. Addition of one drop of the DAB chromogen (Reagent B2 or I2) to one drop of Buffer/Substrate Buffer (Reagent B1 or I1), as is carried out with most Invitrogen detection kits, decreases the pH from ˜7 to pH 6.5-7.1. Addition of two drops of the DAB chromogen (Reagent B2 or I2) to one drop of Buffer/Substrate Buffer (Reagent B1 or I1), as is carried out with the SuperPicture™ kit, decreases the pH to 3.8 to 5.1. Therefore, as shown in Table 1, depending upon the actual mixture of components, the resulting pH may fall well outside of the buffering range possible for a Tris buffer, which buffering range is pH 7.5 to pH 9.0. Without knowing the optimal pH for a particular application, a recommendation could not be made as to an optimal final reaction pH.

Further, as shown in Table 1, the data also indicate that the pH drop is more pronounced with a new lot of Tris, whereas the drop in pH is similar for new and old lots of DAB. Therefore, although there appears to be less buffering capacity in a new lot of Tris as compared to an older lot, instructions for use of the SuperPicture™ kit do not call for adjusting the pH of the final solution. It is assumed that the final pH of the solution is controlled by mixing the appropriate amounts of Tris-HCl and Tris-Base.

The composition of Buffer/Substrate Buffer Reagent B1 is:

| | | |
|---|---|---|
| Description | Qty. Req/Unit | Concentration (M) |

| | | | |
|---|---|---|---|
| Tris-HCl | 0.396 | g | 0.837M |
| Tris-Base | 0.058 | g | 0.160M |
| Reagent Water | 3 | mL | |

The composition of DAB chromogen Reagent B2 is:

| | | |
|---|---|---|
| Description | Qty. Req/Unit | Concentration (M) |

| | | | |
|---|---|---|---|
| 0.1N HCl | 0.15 | mL | 5 mM |
| Methanol | 2.55 | mL | |
| DAB Powder | 0.054 | g | 50 mM |
| Reagent Water | 0.3 | mL | |

DAB contains four amino groups all of which are protonated in the DAB-HCl used to make DAB chromogen Reagent B2.

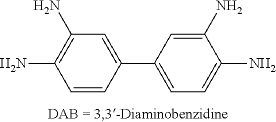

DAB chromogen Reagent B2 contains a total of 205 mM in acid (4×50 mM+5 mM). Under the assumption that one drop is 50 μL, the final concentrations in the working detection reagent when one drop of DAB chromogen Reagent B2 is added are approximately 2.5 mM DAB and approximately 10.25 mM acid. Two drops, as in the SuperPicture™ DAB kit, results in 5 mM DAB and 20.5 mM in acid.

The final concentration of Tris in the Buffer/Substrate Buffer Reagent B1 is approximately 1M. Tris has a pKₐ of 8.06 at 23° C., and a workable buffered pH range between 7.5 and 9.0. Again under the assumption that one drop is 50 μL, the final concentration in the working detection reagent is 50 mM Tris.

As shown in Table 1, the addition of one or two drops of the DAB chromogen Reagent B2 to one drop of Buffer/Substrate Buffer Reagent B1 lowers the pH below the pH range over which Tris functions as a buffer. Therefore, small variations in acid introduced with the DAB solution would be expected to greatly affect the final pH of the mixture.

To ensure that additional acid was not being introduced with DAB chromogen Reagent B2, a pH titration curve was determined for a solution of DAB chromogen Reagent B2. Addition of 100 μL of DAB chromogen Reagent B2 should result in 5×10⁻⁶ moles of DAB and 2.05×10⁻⁵ equivalents of H⁺ ion. The titration data suggested that the solution may contain a small amount of additional acid. DAB chromogen Reagent B2 is formulated in methanol; thus, evaporation of some methanol, which would be expected to result in an increase in concentration of both the DAB and the acid, could explain the higher apparent concentration of acid in the stock.

The effect of Buffer/Substrate Buffer Reagent B1 on the pH titration curve for a solution of DAB chromogen Reagent B2 was also examined. It was observed that the drop in pH upon addition of the DAB solution was less (pH 4.18 versus 3.12) in the presence of Buffer/Substrate Buffer Reagent B1. Without being bound to theory, it is believed that there is an initial titration of the protons introduced with the DAB Chromogen Reagent B2 followed by Tris buffering the pH against further change with subsequent addition of base.

A similar pH titration was measured when 1 M Tris, pH 8 was used in place of Buffer/Substrate Buffer Reagent B1. Replacement of Buffer/Substrate Buffer B1 by 1 M Tris, pH 8 resulted in effective buffering when the DAB chromogen Reagent B2 solution was added.

A similar pH titration was measured when 1 M Tris, pH 7.5 was used in place of Buffer/Substrate Buffer Reagent B1. Replacement of Buffer/Substrate Buffer Reagent B1 by 1 M Tris, pH 7.5 was somewhat less effective than 1 M Tris, pH 8 in buffering when the DAB chromogen Reagent B2 solution was added. Although both Buffer/Substrate Buffer Reagent B1 and 1 M Tris, pH 7.5 solutions are effective at buffering the pH around the pKₐ of Tris, there is a substantial difference in pH in the absence of added base. Specifically, when Buffer/Substrate Buffer Reagent B1 is the buffer, the resulting pH is over 0.6 pH units lower than when 1 M Tris, pH 7.5 is used as the buffer.

All of the above experiments were performed by addition of equal amounts of the Buffer/Substrate Buffer Reagent B1 to DAB Chromogen Reagent B2, as is the case with many Invitrogen detection kits. However, the SuperPicture™ detection kits introduce more DAB and acid by adding twice the amount of Chromogen Reagent B2 compared to Buffer/Substrate Reagent B1. Therefore, the effect of increasing volumes of DAB Chromogen Reagent B2 on the pH of a solution containing 100 μL of Buffer/Substrate Buffer Reagent B1 was investigated. The initial pH of the diluted Buffer/Substrate Buffer Reagent B1 was found to be 7.08. When an equivalent volume of DAB Chromogen Reagent B2 is added (i.e., one drop of each), the pH decreased to 4.0. When twice the volume of Chromogen Reagent B2 is added (i.e., one drop of Buffer/Substrate Buffer Reagent B1, and two drops of DAB Chromogen Reagent B2), the pH decreased to around 3.1.

In the above experiments, the volumes of Buffer/Substrate Buffer Reagent B1 and DAB chromogen Reagent B2 were actually measured. However, it is likely that the use of drops, rather than actually measured volumes, may result in significant differences in the volume of each reagent actually added, thereby resulting in large variation in the reaction pH. Aging of DAB chromogen Reagent B2, which contains 85% methanol, can be expected to lead to increased concentrations of DAB and acid through evaporative loss of methanol, while also contributing to fluctuations in the amount of acid added to the final reaction mixture.

The pH optimum for Horseradish Peroxidase (HRP) reported in the literature varies. The pH optimum for oxidation of DAB by HRP has been reported to be 4.3, with a sharp drop off in activity above and below this pH (see, Herzog, V. and Fahimi, H D. (1973). A New Sensitive Colorimetric Assay for Peroxidase Using 3,3′-Diaminobenzidine as Hydrogen Donor. Analytical Biochemistry 55: 554-562). This extreme dependence of DAB oxidation on pH is consistent with the problems described herein observed upon use of newer lots of Tris.

In light of the data obtained in support of the present invention, it was deemed advisable to determine the optimal pH for the SuperPicture™ kit and then select a buffer that has a pKa in this range to be formulated at a concentration sufficient to control the final pH. Further, it was deemed advisable to reformulate the DAB chromogen Reagent B2 in order to i) remove the methanol from the Reagent and (based on results described below) ii) include a chelating agent to reduce metal-oxygen induced oxidation, and likely concomitant color change and/or precipitation, of the DAB.

### Enhancement of Signals in CISH

Work was also initiated to identify buffer formulations that would result in stronger CISH signals. The incorporation of nitrogenous ligands has been reported to interact with HRP thereby increasing its activity and extending the pH optimum for the reaction (see, Kuo, Che-Fu and Fridovich, Irwin. (1988). Stimulation of the Activity of Horseradish Peroxidase by Nitrogenous Compounds. Journal of Biological Chemistry. 263, No. 8: 3811-3817; Fridovich, Irwin. (1963). The Stimulation of Horseradish Peroxidase by Nitrogenous Ligands. Journal of Biological Chemistry. 283, No. 12: 3821-3927; and Claiborne, Al and Fridovich, Irwin. (1979). Chemical and Enzymatic Intermediates in the Peroxidation of o-Diansidine by Horseradish Peroxidase. 2. Evidence for a Substrate Radical-Enzyme Complex and Its Reaction with Nucleophiles. Biochemistry. 18, No. 11: 2329-2335). Accordingly, a series of buffer compositions covering the pH range from pH 3.0 to 7.5, with and without the addition of imidazole, were examined. CISH was performed on non-amplified breast cancer tissue using the digoxigenin-labeled HER2 probe. Development of HRP was performed with DAB as the substrate using the reaction mixtures outlined in Table 2. The final pH of the buffers following addition of the DAB was measured as was the color of the solution (Table 2).

Many of the solutions containing DAB and hydrogen peroxide turned various shades of brown (Table 2). The brown coloration was most intense in the solutions containing sodium acetate (Solutions 5, 6, 9 and 10 in Table 2). The intensity of the brown coloration increased with time. The brown coloration seen in many of the solutions was more intense at the interphase between the liquid and the atmosphere, thereby suggesting that the mechanism of color formation involved oxidation in the presence of molecular oxygen. With overnight incubation, precipitation was noted even when no HRP was present.

The resulting CISH intensity scores obtained when each of the buffers were used for HRP color development are summarized in Table 3. The use of acetate-containing buffers resulted in very high backgrounds. Incorporation of imidazole in the HRP reaction increased the signal intensity. Optimal HER2 CISH signals were obtained in buffers 11, 12, 15, 16, 20 and 22 (Table 3).

Oxidation frequently is accelerated by the presence of metals. Therefore, it seemed reasonable that the addition of chelating agents to the DAB solution might reduce the formation of the brown product. Incubation of DAB and hydrogen peroxide in acetate-containing buffers for prolonged periods was observed to result in the formation of a brown precipitate Addition of 0.8 mM DTPA was found to prevent formation of brown color as well as the development of a precipitate. These results suggest that acetate-containing buffers may contain a sufficient amount of trace metals such that, in combination with oxygen, oxidation of DAB is promoted. Addition of HRP to solutions containing 165 mM Acetate, pH 4, 2.0 mM DAB, 0.8 mM DTPA and 0.025% hydrogen peroxide resulted in the formation of a large precipitate, indicating that addition of the DTPA did not inhibit the enzymatic reaction.

The effect of three different buffers (MES, HEPES and Tris), the presence of imidazole, and the presence of DTPA in the final HRP color development step were examined for HER2 CISH detection. The resulting data are summarized in Table 4. Buffers containing MES or HEPES resulted in very strong CISH HER2 signals. The presence of higher concentrations of Tris showed stronger, more consistent signals than the Tris concentration typically used in Invitrogen HER2 CISH kits. Incorporation of imidazole resulted in more consistent and darker CISH signals. The presence of DTPA in the buffer did not negatively impact the signal intensity or increase background.

A broader range of DTPA and imidazole concentrations were evaluated in Tris and HEPES buffers. The resulting data are summarized in Table 5. The addition of DTPA reduced the amount of precipitate that formed in the final reaction mixture (containing substrate, buffer and hydrogen peroxide) without having a negative impact on the intensity of the final CISH signal. The presence of imidazole increased the signal intensity in all buffer conditions. The presence of DTPA up to 9 mM concentration did not negatively impact signal intensity. Therefore, the addition of DTPA results in a more stable final reaction mixture (containing buffer, hydrogen peroxide and DAB) without having a negative impact on CISH signal intensity.

### Formulation of a Stable DAB Solution

Two problems were sought to be overcome with a typical DAB formulation as presented by an Invitrogen DAB formulation. First, storage of such a DAB formulation results in sporadic oxidation and precipitation of DAB; DAB-containing vials which show intense color and those that show some precipitation result in increased backgrounds when used in CISH. Second, formulation of a reaction mixture consisting of Buffer/Substrate Buffer (Reagent B1 or I1), DAB chromogen (Reagent B2 or I2), and hydrogen peroxide (Reagent B3 or I3) results in varying rates of DAB oxidation and precipitation following mixing of the three components. Typically, the presence of intense coloration and/or precipitation resulting from oxidation also contributes to increased background when used in CISH. Because this oxidation occurs as a function of time, typical Invitrogen kits have recommended using the reaction mixture essentially immediately after mixing the three aforementioned components (i.e., Reagent B1 or I1+Reagent B2 or I2+Reagent B3 or I3). Requiring such immediate use of the reaction mixture is clearly disadvantageous for automation. Formulation of a more stable reaction mixture would reasonably be expected to result in a more robust kit as well as increased compatibility with automation.

The absorbance spectrum of DAB formulated in water shows a strong absorbance in the UV region of the spectrum with a maximum absorption around 270 nm. Upon storage, there is an increase in brown coloration with a corresponding increase in absorbance in the 465 nm to 520 nm region of the spectrum. These peaks are likely due to the formation of DAB oxidation products. Upon addition of imidazole to 100 mM concentration and hydrogen peroxide to 0.03%, there is a splitting of the UV peak with resulting absorption maxima around 270 nm and 310 nm. In addition, the visible peak shifts from an absorption maximum of 520 nm to around 460 nm. Following incubation of the mixture in the presence of HRP there is a loss of both the visible and UV peaks due to precipitation of the DAB.

The absorbance spectra of a DAB reaction mixture containing DAB, Tris (pH 7.4) and hydrogen peroxide were measured upon incubation at room temperature. After 14 hours of incubation, conjugate containing HRP was added and the mixture was incubated for an additional 5 minutes. The absorbances at 280 nm and 307 nm that were observed are similar to those described when DAB is incubated in water. Also similar to what was found when DAB is incubated in water are absorbance peaks that appear in the range of 465 nm to 478 nm and increase upon incubation at room temperature.

For purposes of the present invention, the absorbance at 465 nm to 520 nm was used to monitor the appearance of the oxidation product(s) of DAB. The absorbance at 280 nm or 307 nm was used to monitor the loss of DAB due to precipitation.

The effect of a series of additives on the stability of DAB formulated in 100 mM Tris, pH 8; 100 mM HEPES, pH 7.4; 100 mM MES, pH 6.5; and 100 mM Imidazole were evaluated based on color changes and the appearance of precipitation upon incubation at 37° C. (Table 6 and Table 7). Loss of DAB through precipitation was monitored by the absorbance at 280 nm (Table 8) while DAB oxidation was monitored by the absorbance at 478 nm (Table 9). With the exception of ascorbic acid and EGTA, most of the additives resulted in reduced DAB precipitation upon incubation at 37° C. (Table 8). Depending on the buffer, the additives propylene glycol, acetonitrile, ribose and DTPA reduced the background oxidation rate of DAB when incubated at elevated temperature (Table 9). The effect of the additives on HRP activity was evaluated following addition of Anti-Mouse HRP Polymer conjugate. The addition of metals has been reported to increase the intensity of the DAB signal generated from the HRP reaction. However, the addition of metals (cobalt, copper and magnesium) resulted in significant precipitation of DAB prior to the addition of enzyme.

The effect of a series of additives on DAB stability when formulated in a wider range of buffers and pH conditions was evaluated spectrophotometrically. Under most of the conditions examined, there was little visible precipitation of DAB with corresponding little loss of UV absorbance (Table 10). The significant loss of absorbance at 280 nm resulting from DAB precipitation upon addition of HRP suggested that the enzyme remained active in all evaluated buffers. Oxidation of DAB was monitored by increased absorbance at 520 nm (Table 11). In general, the incorporation of chelating agents (i.e., DTPA, EDTA, EGTA, 1,10-phenanthroline or diethylene-triaminepentamethylenephosphonic acid) reduced DAB oxidation rates in many of the buffers. With some of the buffers, the additives ascorbic acid, glycerol, and ribose also had a protective effect.

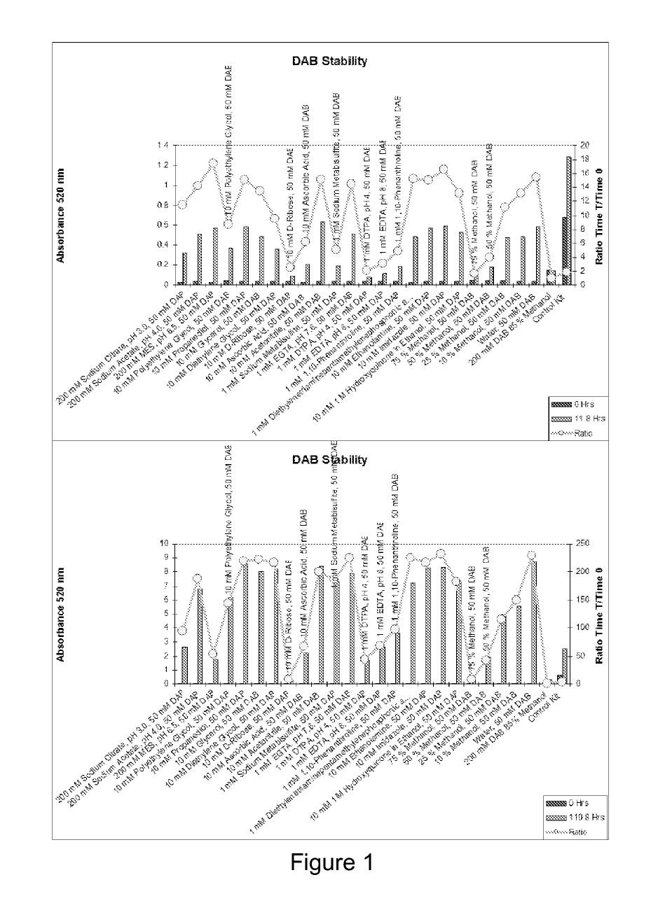

When DAB is formulated in water, there is a 230-fold increase in absorbance at 520 nm upon incubation at 37° C. for 5 days (Table 12). Formulation in 85% methanol had the greatest effect in preventing DAB oxidation (Table 12). The protective effect of methanol shows a dose dependence (Table 12). D-ribose, ascorbate and the chelating agents DTPA, EDTA and EGTA had the greatest effect in reducing DAB oxidation. Upon storage, solutions containing ascorbate and D-ribose turned orange resulting in modification of the corresponding absorption spectra. FIG. 1 demonstrates the protective effect of methanol, D-ribose, ascorbic acid, and the chelating agents DTPA, EDTA, and 1,10-phenanthroline at 11.8 and 110.8 hours.

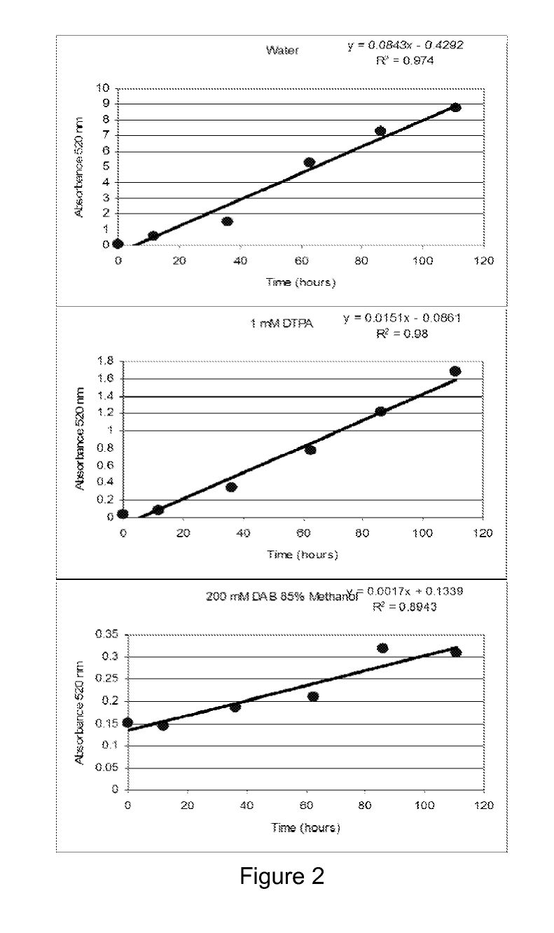

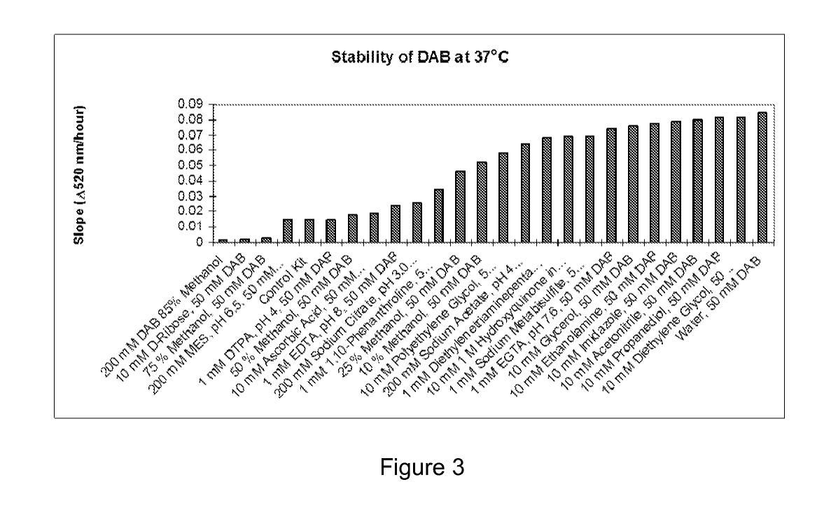

By plotting the increase in absorbance at 520 nm with time (FIG. 2), an initial rate of oxidation can be derived from the slope of the linear regression line (Table 13). Plotting the rate of 520 nm absorbance increase and ordering the values demonstrated that the most effective protective agents are methanol, D-ribose, DTPA, EDTA and 1,10-phenanthroline (FIG. 3).

A study to examine the ability of DTPA, EDTA, 1,10-phenanthroline, D-ribose, polyethylene glycol, sodium metabisulfite and ascorbate concentration to decrease DAB oxidation was performed (Table 14). Optimum protection occurred at 2 mM DTPA, 20 mM D-ribose, 50 mM polyethylene glycol, 5 mM sodium metabisulfite and at all ascorbate concentrations.

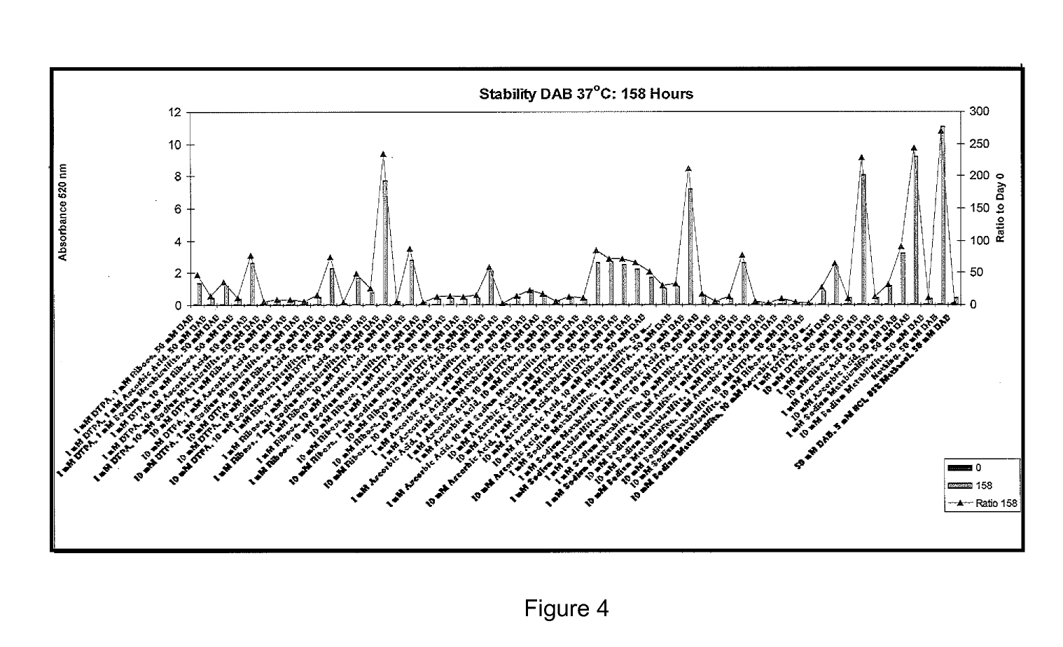

Combinations of agents were evaluated at two concentrations (Table 15 and Table 16). Combinations containing DTPA, sodium metabisulfite and/or D-ribose showed the greatest protective effect. Formulations containing 10 mM sodium metabisulfite contained visible precipitation, while those containing D-ribose or ascorbate became dark orange or brown (Table 16). As a result of the color change, D-ribose and ascorbate were avoided. The absorbance at 520 nm of each of the buffers is summarized in FIG. 4.

Additional formulations and stability studies revealed that sodium metabisulfite tended to form precipitates even when formulated at 1 mM concentration. Sodium sulfite was evaluated as a potential alternative to sodium metabisulfite (Table 17). Whereas concentrations of sodium metabisulfite above 1 mM resulted in precipitation, concentrations of sodium sulfite as high as 50 mM did not show significant precipitation (Table 17). Concentrations of sodium sulfite in the range of 1 mM to 50 mM displayed a protective effect against DAB oxidation similar to that seen with 1-10 mM sodium metabisulfite.

Additional DAB stabilizers were also tested. Polyethylene glycol (PEG), propylene glycol (PG), dimethylsulfoxide (DMSO), glycerol, and 1-methyl-2-pyrrolidone were added to a 50 mM DAB solution containing 10 mM DTPA and 1 mM sodium metabisulfite. The addition of propylene glycol and PEG had a positive effect on DAB stability, reducing the absorbance maximum more than the DAB solution without these additives. Addition of DMSO, glycerol or 1-methyl-2-pyrrolidone had a negative effect as absorbance values increased compared to the control solution. Higher concentrations of PG were also tested. DAB solutions containing 60% PG and 70% PG displayed significantly reduced absorbance maxima compared to DAB solutions without PG and a DAB/85% Methanol solution over the same time course.

Several modifications of the final components were retested to ensure robustness. Sodium sulfite was again compared to sodium metabisulfite. The performance of two DAB solutions formulated with either sodium metabisulfite or sodium sulfite was compared using IHC. Replacement of sodium metabisulfite with sodium sulfite in the DAB formulation slightly improved signal intensity although both formulations outperformed the current Invitrogen-Zymed DAB solution. Based on the results obtained, DAB was formulated with 10 mM DTPA, 1 mM sodium sulfite and 65% propylene glycol.

Notwithstanding the observation that prototypical DAB formulations containing sodium sulfite, DTPA, and propylene glycol display greatly improved DAB stability, the concomitant staining intensity thereof was lower than that of DAB formulations from many competitors. A higher staining intensity was produced by increasing the concentration of DAB in the stock solution (Table 18). The prototypical DAB chromogen solution containing 5 mM DAB outperformed 3 of 4 competitor products when tested by IHC. Higher concentrations of DAB resulted in an increase in signal intensity, but also generated an increase in background staining.

Based on the data obtained, a final formulation of DAB including 200 mM DAB, 20 mM HCl, 10 mM DTPA, 1 mM Sodium Sulfite, and 65% Propylene Glycol was deemed optimal.

### Formulation of a Stable Hydrogen Peroxide Solution

Another improvement sought to be developed for Invitrogen detection kits, which kits have heretofore consisted of three components (see paragraph [0033] above), is a two-component system, including a DAB chromogen component and a hydrogen peroxide component, in which the hydrogen peroxide component would be stably formulated in a reaction buffer.

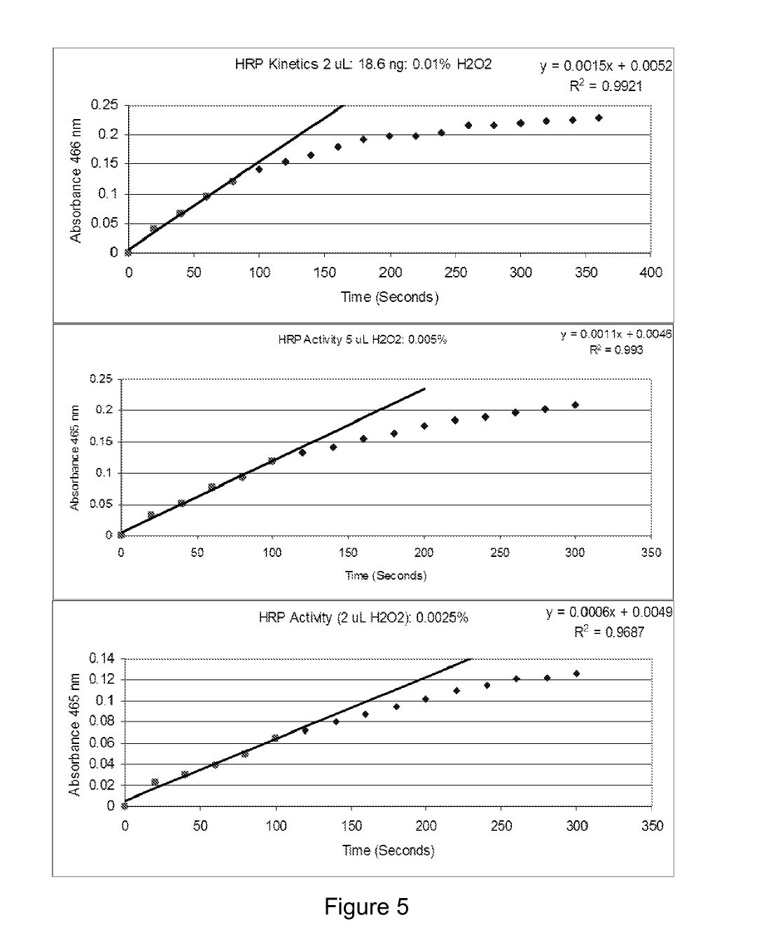

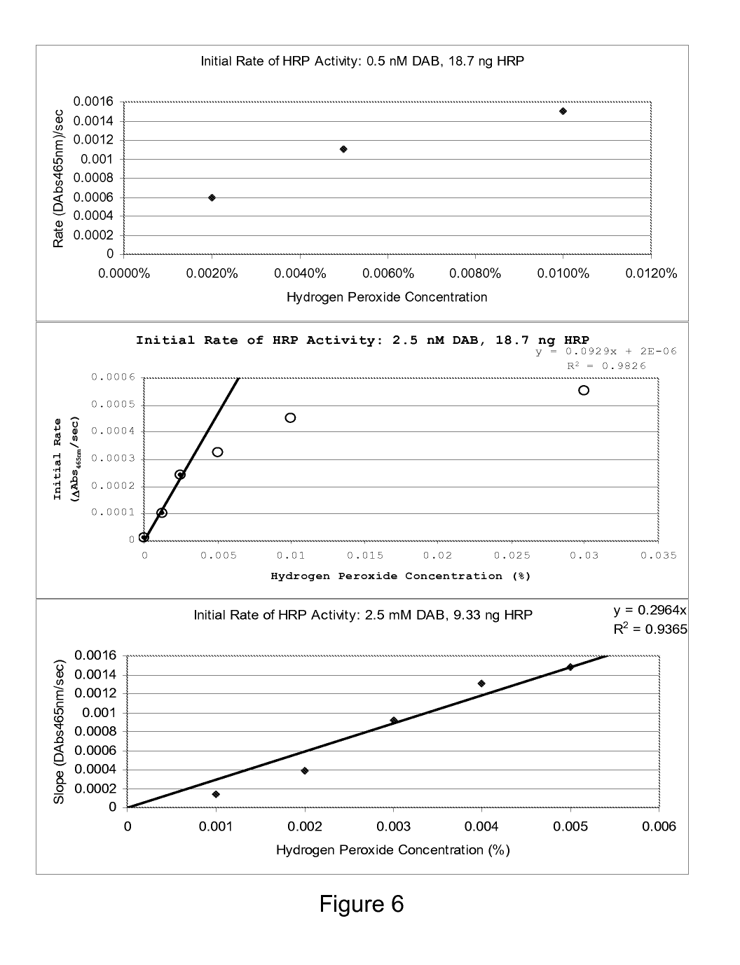

An assay for HRP activity was developed based on Herzog and Fahimi (see, Herzog, V. and Fahimi, H D. (1973). A New Sensitive Colorimetric Assay for Peroxidase Using 3,3′-Diaminobenzidine as Hydrogen Donor. Analytical Biochemistry 55: 554-562) to indirectly assay for active hydrogen peroxide concentration (FIG. 5 and FIG. 6). Incorporation of imidazole into HRP reaction buffers has been reported to increase enzymatic activity and extend the pH range over which the enzyme is active (see, Kuo, Che-Fu and Fridovich, Irwin. (1988). Stimulation of the Activity of Horseradish Peroxidase by Nitrogenous Compounds. Journal of Biological Chemistry. 263, No. 8: 3811-3817; Fridovich, Irwin. (1963). The Stimulation of Horseradish Peroxidase by Nitrogenous Ligands. Journal of Biological Chemistry. 283, No. 12: 3821-3927; and Claiborne, Al and Fridovich, Irwin. (1979). Chemical and Enzymatic Intermediates in the Peroxidation of o-Diansidine by Horseradish Peroxidase. 2. Evidence for a Substrate Radical-Enzyme Complex and Its Reaction with Nucleophiles. Biochemistry. 18, No. 11: 2329-2335.) The HRP activity using Invitrogen kit reagents was found to be 0.0039 Abs₄₆₅ₙₘ/sec (Table 19). In the presence of 100 mM imidazole, the enzymatic rate increased to 0.00765 Abs₄₆₅ₙₘ/sec. Increasing the imidazole concentration to 200 mM increased the rate only slightly, i.e., to 0.00830 Abs₄₆₅ₙₘ/sec. The effect of hydrogen peroxide concentration on HRP activity was measured in the presence of imidazole (Table 19). Increasing the hydrogen peroxide concentration from 0.001% to 0.015% resulted in increased HRP activity. The HRP activity was similar at both 0.015% and 0.030% hydrogen peroxide. Therefore, 0.015% was selected as the concentration in the final buffer.

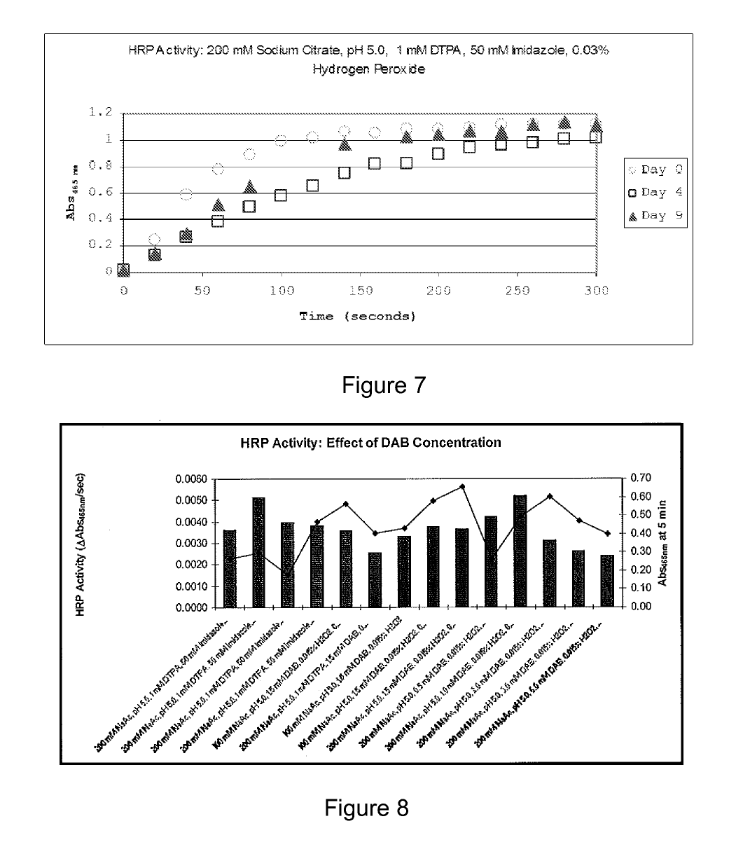

With respect to pH, the greatest activity of HRP is found with acidic buffers (Table 20). With the exception of 200 mM MES, pH 6.5, 200 mM HEPES, pH 7.4, and 50 mM Imidazole, hydrogen peroxide appears stable when stored at 37° C. for 112 hours (Table 20). Based on the activity of HRP and hydrogen peroxide stability, focus was placed on citrate and acetate buffers in the pH range of 4.0 to 5.0. A reaction mixture containing 200 mM Sodium Citrate, pH 5.0, 1 mM DTPA, 50 mM imidazole, and 0.03% hydrogen peroxide was observed to lose only a small portion of its activity when stored at 37° C. for 9 days (FIG. 7).

The effect of DAB concentration on HRP activity was evaluated in buffers containing 200 mM sodium acetate, pH 5.0, 1 mM DTPA, 50 mM imidazole and 0.015% hydrogen peroxide (Table 21, FIG. 8). There was little effect on both HRP activity and total amount of oxidized signal over DAB concentrations ranging between 0.5 mM to 1.5 mM. In the absence of imidazole and DTPA, HRP activity decreased slightly at concentrations between 2.0 mM to 6.0 mM while the total amount of signal was lower at 0.5 mM DAB (Table 21, FIG. 8). Under some conditions, increasing the DAB concentration to 6 mM resulted in substantial decrease in HRP activity with smaller decrease in the total amount of DAB signals.

Although the HRP rate increased with increasing imidazole concentration, the total amount of signal seen at 5 minutes remained constant (Table 22). The effect of imidazole concentration was investigated using three different buffering systems: 0 to 1 M imidazole, and in the absence of imidazole either 50 mM Tris at pH 8 or 100 mM phosphate at pH 6 and 7. As was seen with acetate buffers, the enzymatic HRP rate was affected more than the total amount of DAB signal. The HRP rate reached a maximum at around 100 mM to 200 mM imidazole. In contrast, the DAB signal reached a maximum value at 50 mM imidazole.

The effect of a few other additives in the reaction buffer was also investigated (Table 23). Although higher concentrations of dextran sulfate increased the overall catalytic rate of HRP, the total amount of signal did not increase.

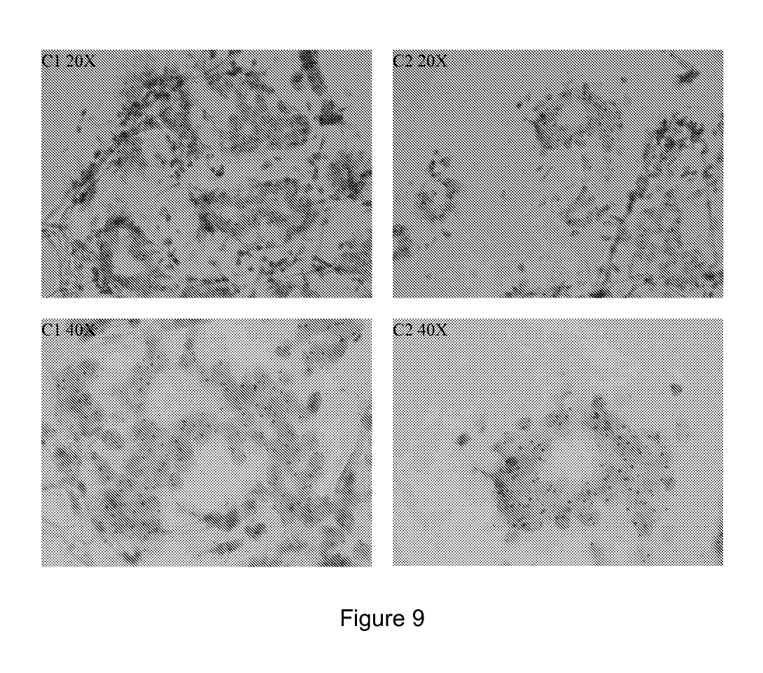

The stability of the hydrogen peroxide-containing buffer was tested in CISH. Reaction buffer consisting of 200 mM sodium acetate, pH 5.0, 1 mM DTPA, 50 mM Imidazole and 0.03% hydrogen peroxide was stored at 37° C. for 27 days. When used as the detection buffer in CISH with 50 mM DAB, 10 mM DTPA, 65% propylene glycol and 10 mM sodium sulfite, the signal intensity was stronger than that seen using the control reagents (FIG. 9).

Based on the data obtained, a final formulation of stable reaction buffer including 200 mM Sodium Acetate, pH 5.5, 1 mM DTPA, 50 mM Imidazole, and 0.03% Hydrogen Peroxide was deemed optimal.

A detailed description of the invention having been provided above, the following examples are given for the purpose of illustrating the invention and shall not be construed as being a limitation on the scope of the invention or claims.

### EXAMPLES

### Evaluation of Competitive Reagents

Several competitive DAB-based HRP detection systems were analyzed. In initial screening of competitive reagents, the most intense signals were obtained with the PowerVision™ Plus and Vector ImmPACT™ reagents. The pH of the components from a number of the kits were measured (Table 24).

The effect of several competitive detection systems on HRP activity was assessed (Table 25). The PowerVision™ and ImmPACT™ DAB kits displayed significantly higher HRP catalytic activity than that found with standard Invitrogen reagents. With the PowerVision™ buffer system, 1.5 mM DAB was found to be less effective than the PowerVision™ chromogen. In contrast, 200 mM NaOAc, pH 5.0, 1 mM DTPA, 50 mM Imidazole, 0.03% H₂O₂ was found to be more active than the PowerVision™ buffer system with the PowerVision™ chromogen. The ImmPACT™ buffer system was found to be more active with 1.5 mM DAB than a 200 mM sodium acetate, pH 5.0, 1 mM DTPA, 50 mM Imidazole, 0.03% H₂O₂ formulation of the present invention.

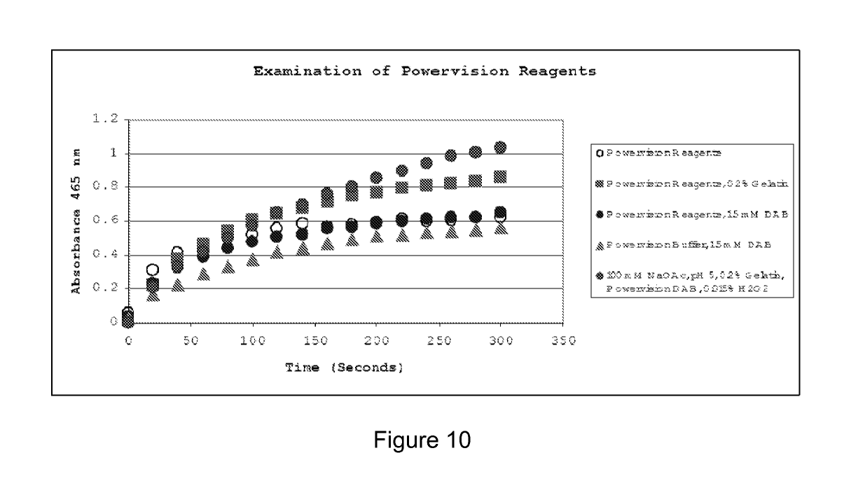

Each of the components of the PowerVision™ detection system was assessed in the HRP assay (FIG. 10). Inclusion of 0.2% gelatin as is typically done in the assay increases the initial increase in signal as well as the total signal generated after 5 minutes. Replacing the PowerVision™ chromogen with 1.5 mM DAB resulted in a decrease in both the initial rate and the total DAB product generated. Replacement of the PowerVision™ buffer system with 100 mM sodium acetate, pH 5 and 0.015% H₂O₂ significantly improved the total signal and prolonged the duration of signal increase (FIG. 10).

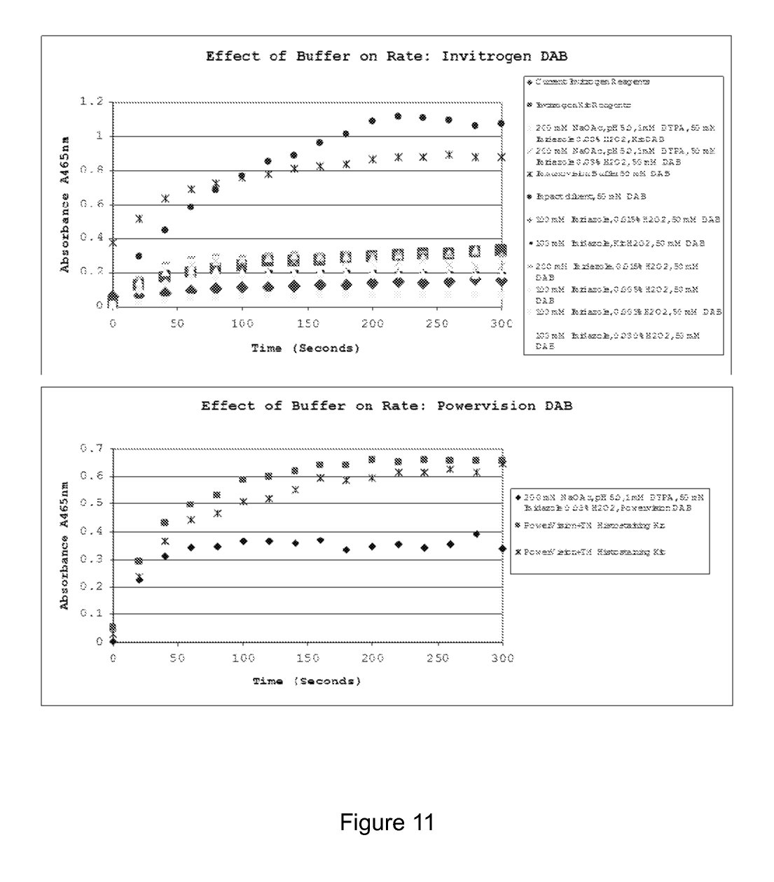

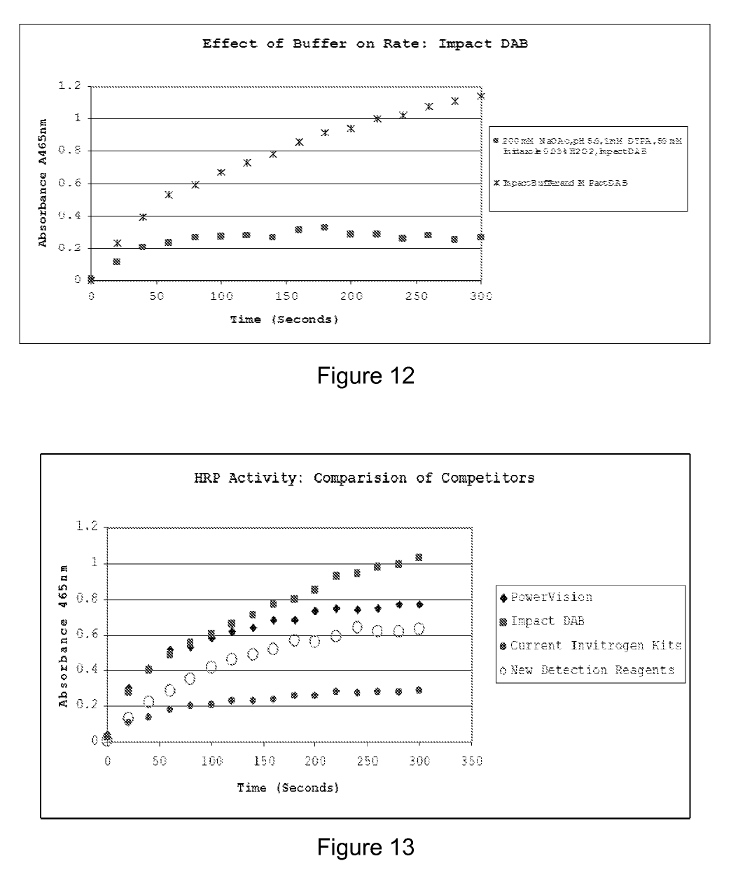

The absorption spectrum of the PowerVision™ Plus chromogen reagent suggested that this chromogen reagent utilizes a modified DAB. Evaluation of the ImmPACT™ and ImmunoVision™ buffers revealed their superiority over the formulation in current Invitrogen CISH kits. Using imidazole in the buffer did not improve the enzymatic activity to the level found in the two competitive kits (FIG. 11). The improved performance appears related to the buffer formulation (FIG. 12). The new formulation of reagents of the present invention (“New Detection Reagents” in FIG. 13) shows superior performance to the formulation in heretofore current Invitrogen CISH kits, approaching that seen in the PowerVision™ and ImmPACT™ DAB detection kits (FIG. 13).

### Design Verification and Validation

The final formulations of the paired DAB chromogen (i.e., 200 mM DAB, 20 mM HCl, 10 mM DTPA, 1 mM Sodium Sulfite, and 65% Propylene Glycol) and Hydrogen Peroxide Buffer (i.e., 200 mM Sodium Acetate, pH 5.5, 50 mM Imidazole, 1 mM DTPA, and 0.03% Hydrogen Peroxide) solutions, referred to herein as “Next Generation Detection Reagents,” were tested by IHC for reproducibility, robustness of the DAB source material, general stability of the components, and equivalency to current Invitrogen products and competitor offerings.

### Reproducibility

To evaluate the repeatability and reproducibility of the Next Generation Detection reagents, as provided by the present invention, multiple tissue types and protein targets were tested to assess for broad spectrum functionality (Table 26, Table 27, Table 28, and Table 30). Using IHC, three independently manufactured R&D lots of the final formulation of the Next Generation DAB chromogen and Hydrogen Peroxide Buffer were compared to assess lot-to-lot reproducibility (Table 26). With regard to signal intensity, each lot produced similar results (signal intensity score varied <0.5 units) when tested by IHC using two different primary antibodies. Day-to-day reproducibility (Table 28) and intra-run reproducibility (Table 27) were also assessed and exceeded the Design Input Specifications for this program. Specifically, using the Next Generation DAB chromogen and Hydrogen Peroxide Buffer, 90% of the samples (n=10) gave the same signal intensity when run on 3 separate days and 100% of the samples (n=12) gave the same signal intensity (≦0.43) when run in triplicate in the same assay.

### Robustness

Four sources of DAB from different manufacturers were tested to determine the robustness of the new DAB detection system (Table 29). Four identical formulations of Next Generation DAB chromogen solution were prepared with each DAB source and compared by IHC. Regardless of the DAB source material, the signal intensity was reproducible and consistent. Each of the four Next Generation DAB formulations outperformed the Invitrogen-Zymed DAB chromogen and buffer currently supplied in the Invitrogen SuperPicture™ Polymer Detection Kit (87-9663).

### Stability

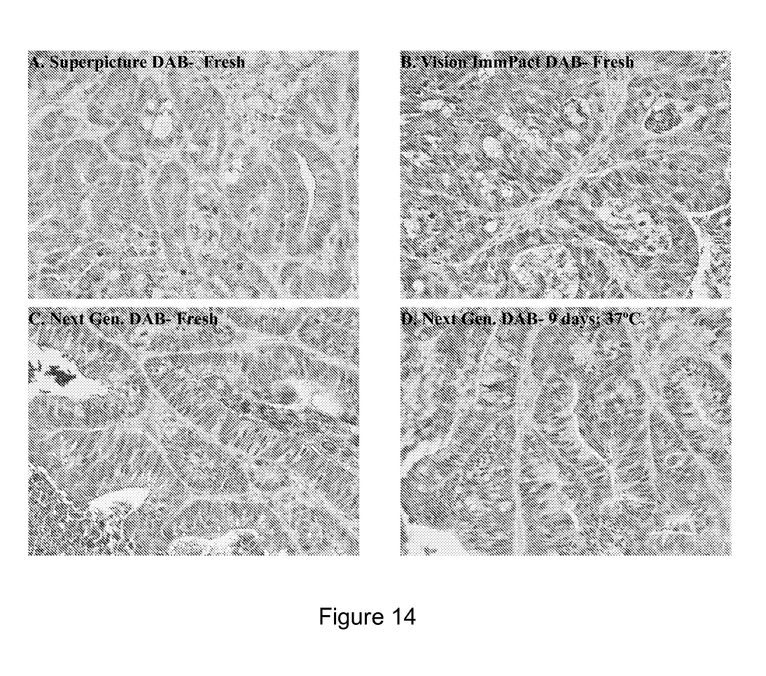

The final formulation of the Next Generation DAB Chromogen and Hydrogen Peroxide Buffer were first tested for long term stability by assessing performance of these formulations after storage at 37° C. for 9 days (comparable to 6 months to 1 year at 4° C.; see, Anderson, Geoffrey and Scott, Milda. (1991). Determination of Product Shelf Life and Activation Energy for Five Drugs of Abuse. Clin. Chem. 37, No. e: 398-402.). As shown in FIG. 14, the Next Generation DAB chromogen incubated for 9 days at 37° C. generated a robust signal compared to freshly prepared Next Generation DAB chromogen and Hydrogen Peroxide Buffer and outperformed the Invitrogen-Zymed DAB chromogen and buffer currently supplied in the Invitrogen SuperPicture™ Polymer Detection Kit. The same DAB solution was tested again at 30 days during competitor comparison testing and produced the same signal intensity as fresh DAB (Table 30). Assuming an activation energy of 20 kcal, the accelerated 30 day stability of the DAB solution is comparable to a real time stability of 2-3 years at 4° C.

### Equivalency/Competitor Comparison Testing

IHC was performed to evaluate the effectiveness of the Next Generation Detection platform versus the Invitrogen SuperPicture™ detection kit and four competitor product lines (Table 30). The Next Generation detection platform consistently outperformed the Invitrogen SuperPicture™ Detection system as evidenced by an increase in mean signal intensity (3.5 vs. 3.18) when compared across 11 tissues using three different primary antibodies. On average, the Next Generation DAB kit outperformed 2 of 4 competitive DAB products and was equivalent to the other two competitive products tested (Table 30).

Each of the above-cited references are hereby incorporated herein by reference as if set forth fully herein.

TABLE 1
pH of mixtures of new and old lots of Buffer/Substrate
Buffer (Reagent B1) and DAB Chromogen (Reagent B2)

pH

| | Old Tris | New Tris |
|---|---|---|
| No DAB | 7.61 | 7.57 |
| Old DAB | | |
| 1 drop | 7.09 | 6.64 |
| 2 drops | 4.71 | 3.77 |
| New DAB | | |
| 1 drop | 7.11 | 6.55 |
| 2 drops | 5.06 | 3.97 |

TABLE 2
Screen of Buffers for CISH.

| | | Color | Final pH |
|---|---|---|---|
| 1 | 166 mM Sodium Citrate pH 3.0, | Light Brown | 3.26 |
| | 0 mM Imidazole, 2 mM, DAB, | | |
| | 0.025% H₂O₂ | | |
| 2 | 160 mM Sodium Citrate pH 3.0, | Light Brown | 3.61 |
| | 40 mM Imidazole, 2 mM, DAB, | | |
| | 0.024% H₂O₂ | | |
| 3 | 166 mM Sodium Citrate pH 4.0, | Light Brown | 4.38 |
| | 0 mM Imidazole, 2 mM, DAB, | | |
| | 0.025% H₂O₂ | | |
| 4 | 160 mM Sodium Citrate pH 4.0, | Light Brown | 4.71 |
| | 40 mM Imidazole, 2 mM, DAB, | | |
| | 0.024% H₂O₂ | | |
| 5 | 166 mM Sodium Acetate pH 4.0, | Dark Brown | 4.03 |
| | 0 mM Imidazole, 2 mM, DAB, | | |
| | 0.025% H₂O₂ | | |
| 6 | 160 mM Sodium Acetate pH 4.0, | Dark Brown | 4.52 |
| | 40 mM Imidazole, 2 mM, DAB, | | |
| | 0.024% H₂O₂ | | |
| 7 | 166 mM Sodium Citrate pH 5.0, | Light Brown | 5.62 |
| | 0 mM Imidazole, 2 mM, DAB, | | |
| | 0.025% H₂O₂ | | |
| 8 | 160 mM Sodium Citrate pH 5.0, | Light Brown | 5.96 |
| | 40 mM Imidazole, 2 mM, DAB, | | |
| | 0.024% H₂O₂ | | |
| 9 | 166 mM Sodium Acetate pH 5.0, | Dark Brown | 5.01 |
| | 0 mM Imidazole, 2 mM, DAB, | | |
| | 0.025% H₂O₂ | | |
| 10 | 160 mM Sodium Acetate pH 5.0, | Dark Brown | 5.62 |
| | 40 mM Imidazole, 2 mM, DAB, | | |
| | 0.024% H₂O₂ | | |
| 11 | 166 mM Sodium Phosphate, pH 6.0, | Light Brown | 6.63 |
| | 0 mM Imidazole, 2 mM, DAB, | | |
| | 0.025% H₂O₂ | | |
| 12 | 160 mM Sodium Phosphate, pH 6.0, | Light Brown | 6.89 |
| | 40 mM Imidazole, 2 mM, DAB, | | |
| | 0.024% H₂O₂ | | |
| 13 | 166 mM Sodium Citrate pH 6.0, | Light Brown | 6.58 |
| | 0 mM Imidazole, 2 mM, DAB, | | |
| | 0.025% H₂O₂ | | |
| 14 | 160 mM Sodium Citrate pH 6.0, | Light Brown | 7.10 |
| | 40 mM Imidazole, 2 mM, DAB, | | |
| | 0.024% H₂O₂ | | |
| 15 | 166 mM MES, pH 6.5, 0 mM | Light Brown | 6.25 |
| | Imidazole, 2 mM, DAB, | | |
| | 0.025% H₂O₂ | | |
| 16 | 160 mM MES, pH 6.5, 40 mM | Light Brown | 6.61 |
| | Imidazole, 2 mM, DAB, | | |
| | 0.024% H₂O₂ | | |
| 17 | 166 mM Sodium Phosphate, pH 7.0, | Light Brown | 7.36 |
| | 0 mM Imidazole, 2 mM, DAB, | | |
| | 0.025% H₂O₂ | | |
| 18 | 160 mM Sodium Phosphate, pH 7.0, | Light Brown | 7.51 |
| | 40 mM Imidazole, 2 mM, DAB, | | |
| | 0.024% H₂O₂ | | |
| 19 | 166 mM HEPES, pH 7.4, 0 mM | Light Brown | 7.15 |
| | Imidazole, 2 mM, DAB, 0.025% H₂O₂ | | |
| 20 | 160 mM HEPES, pH 7.4, 40 mM | Light Brown | 7.30 |
| | Imidazole, 2 mM, DAB, 0.024% H₂O₂ | | |
| 21 | 181 mM Tris pH 8.0, 0 mM | Medium Brown | 7.99 |
| | Imidazole, 2 mM, DAB, | | |
| | 0.027% H₂O₂ | | |
| 22 | 173 mM Tris pH 8.0, 43 mM | Medium Brown | 8.04 |
| | Imidazole, 2 mM, DAB, | | |
| | 0.026% H₂O₂ | | |
| 23 | 166 mM Tris, pH 7.5, 0 mM | Medium Brown | 5.33 |
| | Imidazole, 2 mM, DAB, | | |
| | 0.025% H₂O₂ | | |
| 24 | 160 mM Tris, pH 7.5, 40 mM | Light Brown | 7.50 |
| | Imidazole, 2 mM, DAB, | | |
| | 0.024% H₂O₂ | | |

The indicated buffers were made (1 mL). The mixture was added to develop the chromogenic signal using CISH on nonamplified slides. The final pH of the reagent was measured and the color of the final solution measured. The brown color in the buffers 5, 6, 9 and 10 began forming almost immediately. The solution color formation was noted toward the top of the liquid, suggesting that the color development involved oxidation by oxygen in the air.

TABLE 3
Summary of CISH results using a variety of buffers.

| | | CISH | |
|---|---|---|---|
| | | Inten | |
| 1 | 166 mM Sodium Citrate pH 3.0, | 2.5 | Many cells not |
| | 0 mM Imidazole, 2 mM, DAB, | | labeled. Dots Red |
| | 0.025% H₂O₂ | | in color |
| 2 | 160 mM Sodium Citrate pH 3.0, | 3.0 | More cells |
| | 40 mM Imidazole, 2 mM, DAB | | labeled |
| | 0.024% H₂O₂ | | |
| 3 | 166 mM Sodium Citrate pH 4.0, | 3.0 | |
| | 0 mM Imidazole, 2 mM, DAB, | | |
| | 0.025% H₂O₂ | | |
| 4 | 160 mM Sodium Citrate pH 4.0, | 3.0 | |
| | 40 mM Imidazole, 2 mM, DAB, | | |
| | 0.024% H₂O₂ | | |
| 5 | 166 mM Sodium Acetate pH 4.0, | 3.0 | Some background. |
| | 0 mM Imidazole, 2 mM, DAB, | | Dots red in color. |
| | 0.025% H₂O₂ | | |
| 6 | 160 mM Sodium Acetate pH 4.0, | 3.0 | Some Background. |
| | 40 mM Imidazole, 2 mM, DAB, | | Dots black in |
| | 0.024% H₂O₂ | | color. |
| 7 | 166 mM Sodium Citrate pH 5.0, | 3.0 | Background clear. |
| | 0 mM Imidazole, 2 mM, DAB, | | Dots darker. |
| | 0.025% H₂O₂ | | Better |
| 8 | 160 mM Sodium Citrate pH 5.0, | 3.0 | Blacker, smaller |
| | 40 mM Imidazole, 2 mM, DAB, | | dots. |
| | 0.024% H₂O₂ | | |
| 9 | 166 mM Sodium Acetate pH 5.0, | 3.0 | Very high |
| | 0 mM Imidazole, 2 mM, DAB, | | background. |
| | 0.025% H₂O₂ | | Black dots. |
| 10 | 160 mM Sodium Acetate pH 5.0, | 3.0 | Black dots, very |
| | 40 mM Imidazole, 2 mM, DAB, | | high background. |
| | 0.024% H₂O₂ | | |
| 11 | 166 mM Sodium Phosphate, | 3.5 | Very nice black |
| | pH 6.0, 0 mM Imidazole, 2 mM, | | dots clearly |
| | DAB, 0.025% H₂O₂ | | visible |
| 12 | 160 mM Sodium Phosphate, | 3.5 | Very nice black |
| | pH 6.0, 40 mM Imidazole, 2 mM, | | dots clearly |
| | DAB, 0.024% H₂O₂ | | visible. |
| 13 | 166 mM Sodium Citrate pH 6.0, | 3.0 | Dots not as |
| | 0 mM Imidazole, 2 mM, DAB, | | evident. |
| | 0.025% H₂O₂ | | |
| 14 | 160 mM Sodium Citrate pH 6.0, | 3.0 | Smaller blacker |
| | 40 mM Imidazole, 2 mM, DAB, | | dots. Fewer cells |
| | 0.024% H₂O₂ | | stain. |
| 15 | 166 mM MES, pH 6.5, 0 mM | 3.5 | Dark black dots. |
| | Imidazole, 2 mM, DAB, | | Good. |
| | 0.025% H₂O₂ | | |
| 16 | 160 mM MES, pH 6.5, 40 mM | 3.5 | Dark black dots. |
| | Imidazole, 2 mM, DAB, | | Good. |
| | 0.024% H₂O₂ | | |
| 17 | 166 mM Sodium Phosphate, | 2.5 | Weaker staining. |
| | pH 7.0, 0 mM Imidazole, 2 mM, | | Fewer cells show |
| | DAB, 0.025% H₂O₂ | | dots. |
| 18 | 160 mM Sodium Phosphate, | 3.0 | Weaker staining. |
| | pH 7.0, 40 mM Imidazole, 2 mM, | | Fewer cells show |
| | DAB, 0.024% H₂O₂ | | dots. |
| 19 | 166 mM HEPES, pH 7.4, 0 mM | 3.0 | Fewer cells show |
| | Imidazole, 2 mM, DAB, | | staining. |
| | 0.025% H₂O₂ | | |
| 20 | 160 mM HEPES, pH 7.4, 40 mM | 3.5 | Black dots. Good |
| | Imidazole, 2 mM, DAB, | | staining. |
| | 0.024% H₂O₂ | | |
| 21 | 181 mM Tris pH 8.0, 0 mM | 2.5 | Fewer cells stain. |
| | Imidazole, 2 mM, DAB, | | |
| | 0.027% H₂O₂ | | |
| 22 | 173 mM Tris pH 8.0, 43 mM | 3.5 | Good staining |
| | Imidazole, 2 mM, DAB, | | |
| | 0.026% H₂O₂ | | |
| 23 | 166 mM Tris, pH 7.5, 0 mM | 2.5 | Weak staining. |
| | Imidazole, 2 mM, DAB, | | |
| | 0.025% H₂O₂ | | |
| 24 | 160 mM Tris, pH 7.5, 40 mM | 3.5 | Good staining |
| | Imidazole, 2 mM, DAB, | | |
| | 0.024% H₂O₂ | | |

CISH was performed using the indicated solutions for color development.

TABLE 4
Summary of CISH results using a variety of buffers.

| | Buffer | Inten | Description |
|---|---|---|---|
| 1 | 166 mM, MES pH 6.17, 0 mM | 4 | Areas not staining |
| | Imidazole, 0.0 mM DTPA, | | |
| | 0.025% H₂O₂, 2.1 mM DAB | | |
| 2 | 160 mM, MES pH 6.55, 40 mM | 4 | |
| | Imidazole, 0.0 mM DTPA, | | |
| | 0.024% H₂O₂, 2.0 mM DAB | | |
| 3 | 153 mM, MES pH 6.82, 76 mM | 4 | |
| | Imidazole, 0.0 mM DTPA, | | |
| | 0.023% H₂O₂, 1.9 mM DAB | | |
| 4 | 152 mM, MES pH 6.93, 76 mM | 4 | Slightly Small Dots |
| | Imidazole, 7.6 mM DTPA, | | |
| | 0.023% H₂O₂, 1.9 mM DAB | | |
| 5 | 142 mM, MES pH 7.37, 142 mM | 4 | |
| | Imidazole, 0.0 mM DTPA, | | |
| | 0.021% H₂O₂, 1.8 mM DAB | | |
| 6 | 141 mM, MES pH 7.35, 141 mM | 4 | |
| | Imidazole, 7.1 mM DTPA, | | |
| | 0.021% H₂O₂, 1.8 mM DAB | | |
| 7 | 166 mM, HEPES pH 7.03, 0 mM | 4 | |
| | Imidazole, 0.0 mM DTPA, | | |
| | 0.025% H₂O₂, 2.1 mM DAB | | |
| 8 | 160 mM, HEPES pH 7.2, 40 mM | 4 | Very Nice |
| | Imidazole, 0.0 mM DTPA, | | |
| | 0.024% H₂O₂, 2.0 mM DAB | | |
| 9 | 153 mM, HEPES pH 7.34, 76 mM | 4 | |
| | Imidazole, 0.0 mM DTPA, | | |
| | 0.023% H₂O₂, 1.9 mM DAB | | |
| 10 | 152 mM, HEPES pH 7.35, 76 mM | 4 | |
| | Imidazole, 7.6 mM DTPA, | | |
| | 0.023% H₂O₂, 1.9 mM DAB | | |
| 11 | 142 mM, HEPES pH 7.54, 142 mM | 4 | |
| | Imidazole, 0.0 mM DTPA, | | |
| | 0.021% H₂O₂, 1.8 mM DAB | | |
| 12 | 141 mM, HEPES pH 7.48, 141 mM | 4 | Very Nice |
| | Imidazole, 7.1 mM DTPA, | | |
| | 0.021% H₂O₂, 1.8 mM DAB | | |
| 13 | 181 mM, Tris pH 7.93, 0 mM | 4 | Very Nice |
| | Imidazole, 0.0 mM DTPA, | | |
| | 0.027% H₂O₂, 2.3 mM DAB | | |
| 14 | 173 mM, Tris pH 8, 43 mM | 4 | Very Nice |
| | Imidazole, 0.0 mM DTPA, | | |
| | 0.026% H₂O₂, 2.2 mM DAB | | |
| 15 | 166 mM, Tris pH 8.05, 83 mM | 4 | Very Nice |
| | Imidazole, 0.0 mM DTPA, | | |
| | 0.025% H₂O₂, 2.1 mM DAB | | |
| 16 | 165 mM, Tris pH 8.06, 82 mM | 3.5 | |
| | Imidazole, 8.3 mM DTPA, | | |
| | 0.025% H₂O₂, 2.1 mM DAB | | |
| 17 | 153 mM, Tris pH 8.11, 153 mM | 3.5 | Dots black |
| | Imidazole, 0.0 mM DTPA, | | |
| | 0.023% H₂O₂, 1.9 mM DAB | | |
| 18 | 152 mM, Tris pH 8.12, 152 mM | 3.5 | |
| | Imidazole, 7.6 mM DTPA, | | |
| | 0.023% H₂O₂, 1.9 mM DAB | | |
| 19 | 47 mM, Tris pH 4.09, 0 mM | 3.5 | |
| | Imidazole, 0.0 mM DTPA, | | |
| | 0.029% H₂O₂, 2.4 mM DAB | | |
| 20 | 45 mM, Tris pH 7.32, 45 mM | 3.5 | |
| | Imidazole, 0.0 mM DTPA, | | |
| | 0.027% H₂O₂, 2.3 mM DAB | | |
| 21 | 43 mM, Tris pH 7.73, 86 mM | 3 | Not all cells |
| | Imidazole, 0.0 mM DTPA, | | staining |
| | 0.026% H₂O₂, 2.2 mM DAB | | |
| 22 | 43 mM, Tris pH 7.72, 86 mM | 3 | areas not staining |
| | Imidazole, 8.6 mM DTPA, | | |
| | 0.026% H₂O₂, 2.2 mM DAB | | |
| 23 | 40 mM, Tris pH 7.96, 160 mM | 3 | areas not staining |
| | Imidazole, 0.0 mM DTPA, | | |
| | 0.024% H₂O₂, 2.0 mM DAB | | |
| 24 | 39 mM, Tris pH 7.96, 158 mM | 2.5 | Not good |
| | Imidazole, 7.9 mM DTPA, | | haematoxylin |
| | 0.024% H₂O₂, 2.0 mM DAB | | |

CISH was performed using the indicated solutions for color development.

TABLE 5
Screen of Buffers for CISH.

| | | | | Precip |
|---|---|---|---|---|
| | | | CISH | After |
| | | Inten | Signals | 7 hrs |
| 1 | 181 mM Tris pH 7.97, 0 mM | 3.0 | Many cells | Slight |
| | Imidazole, 0.0 mM DTPA, | | not stained | |
| | 0.027% H₂O₂, 2.3 mM DAB | | | |
| 2 | 181 mM Tris pH 8.04, 90 mM | 3.5 | More cells | Slight |
| | Imidazole, 0.0 mM DTPA, | | stained | |
| | 0.027% H₂O₂, 2.3 mM DAB | | | |
| 3 | 181 mM Tris pH 8.04, 90 mM | 3.5 | More cells | None |
| | Imidazole, 0.9 mM DTPA, | | stained | |
| | 0.027% H₂O₂, 2.3 mM DAB | | | |
| 4 | 181 mM Tris pH 8.03, 90 mM | 3.0 | Many Cells | None |
| | Imidazole, 4.5 mM DTPA, | | Not Stained | |
| | 0.027% H₂O₂, 2.3 mM DAB | | | |
| 5 | 181 mM Tris pH 8.01, 90 mM | 3.5 | Most Cells | None |
| | Imidazole, 9.1 mM DTPA, | | Stained | |
| | 0.027% H₂O₂, 2.3 mM DAB | | | |
| 6 | 181 mM Tris pH 8.09, 181 mM | 3.5 | Most Cells | Slight |
| | Imidazole, 0.0 mM DTPA, | | Stained | |
| | 0.027% H₂O₂, 2.3 mM DAB | | | |
| 7 | 181 mM Tris pH 8.17, 363 mM | 3.5 | Most Cells | Slight |
| | Imidazole, 0.0 mM DTPA, | | Stained. | |
| | 0.027% H₂O₂, 2.3 mM DAB | | Small Black | |
| | | | dots | |
| 8 | 181 mM Tris pH 8.24, 545 mM | 3.5 | Most Cells | ++ |
| | Imidazole, 0.0 mM DTPA, | | Stained. | |
| | 0.027% H₂O₂, 2.3 mM DAB | | Small Black | |
| | | | dots | |
| 9 | 181 mM HEPES, pH 7.17, 0 mM | 3.0 | Most Cells | None |
| | Imidazole, 0.0 mM DTPA, | | Stained | |
| | 0.027% H₂O₂, 2.3 mM DAB | | | |
| 10 | 181 mM HEPES, pH 7.39, 90 mM | 3.0 | Most Cells | None |
| | Imidazole, 0.0 mM DTPA, | | Stained | |
| | 0.027% H₂O₂, 2.3 mM DAB | | | |
| 11 | 181 mM HEPES, pH 7.39, 90 mM | 3.5 | Most Cells | None |
| | Imidazole, 0.9 mM DTPA, | | Stained | |
| | 0.027% H₂O₂, 2.3 mM DAB | | | |
| 12 | 181 mM HEPES, pH 7.38, 90 mM | 3.5 | Most Cells | None |
| | Imidazole, 4.5 mM DTPA, | | Stained. | |
| | 0.027% H₂O₂, 2.3 mM DAB | | Small Black | |
| | | | dots | |
| 13 | 181 mM HEPES, pH 7.35, 90 mM | 3.5 | Most Cells | None |
| | Imidazole, 9.1 mM DTPA, | | Stained. | |
| | 0.027% H₂O₂, 2.3 mM DAB | | Small Black | |
| | | | dots | |
| 14 | 181 mM HEPES, pH 7.53, 181 mM | 4.0 | Most Cells | Slight |
| | Imidazole, 0.0 mM DTPA, | | Stained. | |
| | 0.027% H₂O₂, 2.3 mM DAB | | Small Black | |
| | | | dots | |
| 15 | 181 mM HEPES, pH 7.71, 363 mM | 3.0 | Most Cells | Slight |
| | Imidazole, 0.0 mM DTPA, | | Stained. | |
| | 0.027% H₂O₂, 2.3 mM DAB | | Small Black | |
| | | | dots | |
| 16 | 181 mM HEPES, pH 7.82, 545 mM | 4.0 | Most Cells | Slight |
| | Imidazole, 0.0 mM DTPA, | | Stained. | Brown |
| | 0.027% H₂O₂, 2.3 mM DAB | | Small Black | |
| | | | dots | |
| 17 | 45 mM Tris pH 4.67, 0 mM | 3.0 | Black | Color |
| | Imidazole, 0.0 mM DTPA, | | Precipitate | |
| | 0.027% H₂O₂, 2.3 mM DAB | | | |
| 18 | 45 mM Tris pH 7.64, 90 mM | 3.5 | | Slight |
| | Imidazole, 0.0 mM DTPA, | | | |
| | 0.027% H₂O₂, 2.3 mM DAB | | | |
| 19 | 45 mM Tris pH 7.65, 90 mM | 3.5 | | − |
| | Imidazole, 0.9 mM DTPA, | | | |
| | 0.027% H₂O₂, 2.3 mM DAB | | | |
| 20 | 45 mM Tris pH 7.63, 90 mM | 3.5 | | None |
| | Imidazole, 4.5 mM DTPA, | | | |
| | 0.027% H₂O₂, 2.3 mM DAB | | | |
| 21 | 45 mM Tris pH 7.6, 90 mM | 3.5 | | None |
| | Imidazole, 9.1 mM DTPA, | | | |
| | 0.027% H₂O₂, 2.3 mM DAB | | | |
| 22 | 45 mM Tris pH 7.9, 181 mM | 4.0 | | Slight |
| | Imidazole, 0.0 mM DTPA, | | | |
| | 0.027% H₂O₂, 2.3 mM DAB | | | |
| 23 | 45 mM Tris pH 8.11, 363 mM | 4.0 | | + |
| | Imidazole, 0.0 mM DTPA, | | | |
| | 0.027% H₂O₂, 2.3 mM DAB | | | |
| 24 | 45 mM Tris pH 8.25, 545 mM | 3.0 | | + |
| | Imidazole, 0.0 mM DTPA, | | | |
| | 0.027% H₂O₂, 2.3 mM DAB | | | |

The indicated buffers were made (1 mL). The mixture was added to develop the chromogenic signal using CISH on nonamplified slides. The final pH of the reagent was measured and the color of the final solution measured, as indicated. Seven (7) hours after addition, the presence of precipitate was indicated (“Precip”).

TABLE 6
Color characteristics of solutions.

| | | Observed color after addition of: | | |
|---|---|---|---|---|
| | | H₂O₂ | DAB | After 1 hour |
| 1 | 100 mM Tris, pH 8, Nothing, 0.03% H₂O₂, 2.5 mM DAB | | | |
| 2 | 100 mM Tris, pH 8, 50 mM Polyethylene Glycol, 0.03% H₂O₂, 2.5 mM DAB | | | |
| 3 | 100 mM Tris, pH 8, 50 mM Diethylene Glycol, 0.03% H₂O₂, 2.5 mM DAB | | | |
| 4 | 100 mM Tris, pH 8, 50 mM Propanediol, 0.03% H₂O₂, 2.5 mM DAB | | | |
| 5 | 100 mM Tris, pH 8, 50 mM Glycerol, 0.03% H₂O₂, 2.5 mM DAB | | | |
| 6 | 100 mM Tris, pH 8, 50 mM Ribose, 0.03% H₂O₂, 2.5 mM DAB | | | |
| 7 | 100 mM Tris, pH 8, 1 mM EDTA, 0.03% H₂O₂, 2.5 mM DAB | | | |
| 8 | 100 mM Tris, pH 8, 1 mM DTPA, 0.03% H₂O₂, 2.5 mM DAB | | | |
| 9 | 100 mM Tris, pH 8, 1 mM EGTA, 0.03% H₂O₂, 2.5 mM DAB | | | |
| 10 | 100 mM Tris, pH 8, 1 mM 1,10-Phenanthroline, 0.03% H₂O₂, 2.5 mM DAB | | | |
| 11 | 100 mM Tris, pH 8, 1 mM Diethylenetriamine.., 0.03% H₂O₂, 2.5 mM DAB | | | |
| 12 | 100 mM Tris, pH 8, 10 mM Sodium Metabisulfite, 0.03% H₂O₂, 2.5 mM DAB | | | |
| 13 | 100 mM Tris, pH 8, 10 mM Ascorbic Acid, 0.03% H₂O₂, 2.5 mM DAB | | | Dark Yellow |
| 14 | 100 mM Tris, pH 8, 10 mM Acetonitrile, 0.03% H₂O₂, 2.5 mM DAB | | | |
| 15 | 100 mM Tris, pH 8, 1 mM CuCl₂, 0.03% H₂O₂, 2.5 mM DAB | Light Purple | Dark Black | Black |
| 16 | 100 mM Tris, pH 8, 1 mM CoCl₂, 0.03% H₂O₂, 2.5 mM DAB | Light Yellow | Lighter Black | Black |
| 17 | 100 mM Tris, pH 8, 1 mM Mg₂SO₄, 0.03% H₂O₂, 2.5 mM DAB | | | |
| 18 | 100 mM HEPES, pH 7.4, Nothing, 0.03% H₂O₂, 2.5 mM DAB | | | |
| 19 | 100 mM HEPES, pH 7.4, 50 mM Polyethylene Glycol, 0.03% H₂O₂, 2.5 mM DAB | | | |
| 20 | 100 mM HEPES, pH 7.4, 50 mM Diethylene Glycol, 0.03% H₂O₂, 2.5 mM DAB | | | |
| 21 | 100 mM HEPES, pH 7.4, 50 mM Propanediol, 0.03% H₂O₂, 2.5 mM DAB | | | |
| 22 | 100 mM HEPES, pH 7.4, 50 mM Glycerol, 0.03% H₂O₂, 2.5 mM DAB | | | |
| 23 | 100 mM HEPES, pH 7.4, 50 mM Ribose, 0.03% H₂O₂, 2.5 mM DAB | | | |
| 24 | 100 mM HEPES, pH 7.4, 1 mM EDTA, 0.03% H₂O₂, 2.5 mM DAB | | | |
| 25 | 100 mM HEPES, pH 7.4, 1 mM DTPA, 0.03% H₂O₂, 2.5 mM DAB | | | |
| 26 | 100 mM HEPES, pH 7.4, 1 mM EGTA, 0.03% H₂O₂, 2.5 mM DAB | | | |
| 27 | 100 mM HEPES, pH 7.4, 1 mM 1,10-Phenanthroline, 0.03% H₂O₂, 2.5 mM DAB | | | |
| 28 | 100 mM HEPES, pH 7.4, 1 mM Diethylenetriamine.., 0.03% H₂O₂, 2.5 mM DAB | | | |
| 29 | 100 mM HEPES, pH 7.4, 10 mM Sodium Metabisulfite, 0.03% H₂O₂, 2.5 mM DAB | | | Dark Yellow |
| 30 | 100 mM HEPES, pH 7.4, 10 mM Ascorbic Acid, 0.03% H₂O₂, 2.5 mM DAB | | | |
| 31 | 100 mM HEPES, pH 7.4, 10 mM Acetonitrile, 0.03% H₂O₂, 2.5 mM DAB | Light Green | Dark Yellow | Brown, Cloudy |
| 32 | 100 mM HEPES, pH 7.4, 1 mM CuCl₂, 0.03% H₂O₂, 2.5 mM DAB | | | Deep Purple |
| 33 | 100 mM HEPES, pH 7.4, 1 mM CoCl₂, 0.03% H₂O₂, 2.5 mM DAB | | | |
| 34 | 100 mM HEPES, pH 7.4, 1 mM Mg₂SO₄, 0.03% H₂O₂, 2.5 mM DAB | | | |
| 35 | 100 mM MES, pH 6.5, Nothing, 0.03% H₂O₂, 2.5 mM DAB | | | |
| 36 | 100 mM MES, pH 6.5, 50 mM Polyethylene Glycol, 0.03% H₂O₂, 2.5 mM DAB | | | |
| 37 | 100 mM MES, pH 6.5, 50 mM Diethylene Glycol, 0.03% H₂O₂, 2.5 mM DAB | | | |
| 38 | 100 mM MES, pH 6.5, 50 mM Propanediol, 0.03% H₂O₂, 2.5 mM DAB | | | |
| 39 | 100 mM MES, pH 6.5, 50 mM Glycerol, 0.03% H₂O₂, 2.5 mM DAB | | | |
| 40 | 100 mM MES, pH 6.5, 50 mM Ribose, 0.03% H₂O₂, 2.5 mM DAB | | | |
| 41 | 100 mM MES, pH 6.5, 1 mM EDTA, 0.03% H₂O₂, 2.5 mM DAB | | | |
| 42 | 100 mM MES, pH 6.5, 1 mM DTPA, 0.03% H₂O₂, 2.5 mM DAB | | | |
| 43 | 100 mM MES, pH 6.5, 1 mM EGTA, 0.03% H₂O₂, 2.5 mM DAB | | | |
| 44 | 100 mM MES, pH 6.5, 1 mM 1,10-Phenanthroline, 0.03% H₂O₂, 2.5 mM DAB | | | |
| 45 | 100 mM MES, pH 6.5, 1 mM Diethylenetriamine.., 0.03% H₂O₂, 2.5 mM DAB | | | Dark |
| | | | | Yellow |
| 46 | 100 mM MES, pH 6.5, 10 mM Sodium Metabisulfite, 0.03% H₂O₂, 2.5 mM DAB | | | Deep |
| | | | | Yellow |
| 47 | 100 mM MES, pH 6.5, 10 mM Ascorbic Acid, 0.03% H₂O₂, 2.5 mM DAB | Light Yellow | Black | Black with |
| | | | | Precipitate |
| 48 | 100 mM MES, pH 6.5, 10 mM Acetonitrile, 0.03% H₂O₂, 2.5 mM DAB | | | Light Purple |
| 49 | 100 mM MES, pH 6.5, 1 mM CuCl₂, 0.03% H₂O₂, 2.5 mM DAB | | | Light Brown |
| 50 | 100 mM MES, pH 6.5, 1 mM CoCl₂, 0.03% H₂O₂, 2.5 mM DAB | | | Clear |
| 51 | 100 mM MES, pH 6.5, 1 mM Mg₂SO₄, 0.03% H₂O₂, 2.5 mM DAB | | | Light Brown |
| 52 | 100 mM Imidazole, Nothing, 0.03% H₂O₂, 2.5 mM DAB | | | Clear |
| 53 | 100 mM Imidazole, 50 mM Polyethylene Glycol, 0.03% H₂O₂, 2.5 mM DAB | | | Clear |
| 54 | 100 mM Imidazole, 50 mM Diethylene Glycol, 0.03% H₂O₂, 2.5 mM DAB | | | Clear |
| 55 | 100 mM Imidazole, 50 mM Propanediol, 0.03% H₂O₂, 2.5 mM DAB | | | Clear |
| 56 | 100 mM Imidazole, 50 mM Glycerol, 0.03% H₂O₂, 2.5 mM DAB | | | Clear |
| 57 | 100 mM Imidazole, 50 mM Ribose, 0.03% H₂O₂, 2.5 mM DAB | | | Medium Brown |
| 58 | 100 mM Imidazole, 1 mM EDTA, 0.03% H₂O₂, 2.5 mM DAB | | | Clear |
| 59 | 100 mM Imidazole, 1 mM DTPA, 0.03% H₂O₂, 2.5 mM DAB | | | Clear |
| 60 | 100 mM Imidazole, 1 mM EGTA, 0.03% H₂O₂, 2.5 mM DAB | | | Clear |
| 61 | 100 mM Imidazole, 1 mM 1,10-Phenanthroline, 0.03% H₂O₂, 2.5 mM DAB | | | Light Yellow, from top |
| 62 | 100 mM Imidazole, 1 mM Diethylenetriamine.., 0.03% H₂O₂, 2.5 mM DAB | | | Clear |
| 63 | 100 mM Imidazole, 10 mM Sodium Metabisulfite, 0.03% H₂O₂, 2.5 mM DAB | Light Blue | Black | Black |
| 64 | 100 mM Imidazole, 10 mM Ascorbic Acid, 0.03% H₂O₂, 2.5 mM DAB | Light Purple | Black | Black |
| 65 | 100 mM Imidazole, 10 mM Acetonitrile, 0.03% H₂O₂, 2.5 mM DAB | | | Clear |
| 66 | 100 mM Imidazole, 1 mM CuCl₂, 0.03% H₂O₂, 2.5 mM DAB | | | |
| 67 | 100 mM Imidazole, 1 mM CoCl₂, 0.03% H₂O₂, 2.5 mM DAB | | | |
| 68 | 100 mM Imidazole, 1 mM Mg₂SO₄, 0.03% H₂O₂, 2.5 mM DAB | | | |

Buffer and additives were mixed and, when present, color was noted after addition of hydrogen peroxide and DAB. Following one hour of incubation at 37° C., further color changes were also noted.

TABLE 7
Color characteristics of solutions.

| | | 1 Hour | 4 Hours | 16 Hours |
|---|---|---|---|---|
| 1 | 100 mM Tris, pH 8, Nothing, 0.03% H₂O₂, 2.5 mM DAB | Clear | Light Brown | 2+ ppt, slight brown |
| 2 | 100 mM Tris, pH 8, 50 mM Polyethylene Glycol, 0.03% H₂O₂, 2.5 mM DAB | Clear | Light Brown | 1+ ppt, slight brown |
| 3 | 100 mM Tris, pH 8, 50 mM Diethylene Glycol, 0.03% H₂O₂, 2.5 mM DAB | Clear | Light Brown | 2+ ppt, slight brown |
| 4 | 100 mM Tris, pH 8, 50 mM Propanediol, 0.03% H₂O₂, 2.5 mM DAB | Clear | Light Brown | 2+ ppt, slight brown |
| 5 | 100 mM Tris, pH 8, 50 mM Glycerol, 0.03% H₂O₂, 2.5 mM DAB | Clear | Light Brown | 2+ ppt, slight brown |
| 6 | 100 mM Tris, pH 8, 50 mM Ribose, 0.03% H₂O₂, 2.5 mM DAB | Clear | Light Brown | 1+ ppt, slight brown |
| 7 | 100 mM Tris, pH 8, 1 mM EDTA, 0.03% H₂O₂, 2.5 mM DAB | Brown | Slightly | 2+ ppt, slight brown |
| | | | Dark Brown | |
| 8 | 100 mM Tris, pH 8, 1 mM DTPA, 0.03% H₂O₂, 2.5 mM DAB | Clear | Clearer | 2+ ppt, slight brown |
| 9 | 100 mM Tris, pH 8, 1 mM EGTA, 0.03% H₂O₂, 2.5 mM DAB | Brown, Cloudy | Murky Brown; | 4+ ppt, slight brown |
| | | | Ppt | |
| 10 | 100 mM Tris, pH 8, 1 mM 1,10-Phenanthroline, 0.03% H₂O₂, 2.5 mM DAB | Clear | Light Brown | 2+ ppt, slight brown |
| 11 | 100 mM Tris, pH 8, 1 mM Diethylenetriamine.., 0.03% H₂O₂, 2.5 mM DAB | Clear | Very Clear | 1+ ppt, slight brown |
| 12 | 100 mM Tris, pH 8, 10 mM Sodium Metabisulfite, 0.03% H₂O₂, 2.5 mM DAB | Clear | Very Clear | 0 ppt, clear |
| 13 | 100 mM Tris, pH 8, 10 mM Ascorbic Acid, 0.03% H₂O₂, 2.5 mM DAB | Dark Yellow | Dark Yellow | 2+ ppt, yellow |
| 14 | 100 mM Tris, pH 8, 10 mM Acetonitrile, 0.03% H₂O₂, 2.5 mM DAB | Light Brown | Light Brown | 2+ ppt, slight brown |
| 15 | 100 mM Tris, pH 8, 1 mM CuCl₂, 0.03% H₂O₂, 2.5 mM DAB | Black, Large | Black | 4+ ppt, slight black |
| | | Ppt | | |
| 16 | 100 mM Tris, pH 8, 1 mM CoCl₂, 0.03% H₂O₂, 2.5 mM DAB | Black, Ppt | Black | 3+ ppt, slight black |
| 17 | 100 mM Tris, pH 8, 1 mM Mg₂SO₄, 0.03% H₂O₂, 2.5 mM DAB | Clear | Light Brown | 2+ ppt, slight brown |
| 18 | 100 mM HEPES, pH 7.4, Nothing, 0.03% H₂O₂, 2.5 mM DAB | Clear | Very Light | 2+ ppt, slight brown |
| | | | Brown | |
| 19 | 100 mM HEPES, pH 7.4, 50 mM Polyethylene Glycol, 0.03% H₂O₂, 2.5 mM DAB | Clear | Clear | 0 ppt, slight brown |
| 20 | 100 mM HEPES, pH 7.4, 50 mM Diethylene Glycol, 0.03% H₂O₂, 2.5 mM DAB | Clear | Clear | 2+ ppt, slight brown |
| 21 | 100 mM HEPES, pH 7.4, 50 mM Propanediol, 0.03% H₂O₂, 2.5 mM DAB | Clear | Clear | 2+ ppt, slight brown |
| 22 | 100 mM HEPES, pH 7.4, 50 mM Glycerol, 0.03% H₂O₂, 2.5 mM DAB | Clear | Clear | 2+ ppt, slight brown |
| 23 | 100 mM HEPES, pH 7.4, 50 mM Ribose, 0.03% H₂O₂, 2.5 mM DAB | Clear | Clear | No ppt, almost clear |
| 24 | 100 mM HEPES, pH 7.4, 1 mM EDTA, 0.03% H₂O₂, 2.5 mM DAB | Brown | Medium Brown | 2+ ppt, slight brown |
| 25 | 100 mM HEPES, pH 7.4, 1 mM DTPA, 0.03% H₂O₂, 2.5 mM DAB | Clear | Light Brown | 2+ ppt, slight brown |
| 26 | 100 mM HEPES, pH 7.4, 1 mM EGTA, 0.03% H₂O₂, 2.5 mM DAB | Slightly | Dark Murky | 3+ ppt, slight brown |
| | | Darker Brown | Brown | |
| 27 | 100 mM HEPES, pH 7.4, 1 mM 1,10-Phenanthroline, 0.03% H₂O₂, 2.5 mM DAB | Clear | Light Brown | 2+ ppt, slight brown |
| 28 | 100 mM HEPES, pH 7.4, 1 mM Diethylenetriamine.., 0.03% H₂O₂, 2.5 mM DAB | Clear | Clear | 1+ ppt, clear |
| 29 | 100 mM HEPES, pH 7.4, 10 mM Sodium Metabisulfite, 0.03% H₂O₂, 2.5 mM DAB | Clear | Clear | 0 ppt, clear |
| 30 | 100 mM HEPES, pH 7.4, 10 mM Ascorbic Acid, 0.03% H₂O₂, 2.5 mM DAB | Orange | Orange | 2+ ppt, yellow |
| 31 | 100 mM HEPES, pH 7.4, 10 mM Acetonitrile, 0.03% H₂O₂, 2.5 mM DAB | Clear | Very Light | 1+ ppt, slight brown |
| | | | Brown | |
| 32 | 100 mM HEPES, pH 7.4, 1 mM CuCl₂, 0.03% H₂O₂, 2.5 mM DAB | Murky Brown | Murky Green | 4+ ppt, light yellow |
| | | | Brown | |
| 33 | 100 mM HEPES, pH 7.4, 1 mM CoCl₂, 0.03% H₂O₂, 2.5 mM DAB | Black | Black Large | 4+ ppt, black |
| | | | Ppt | |
| 34 | 100 mM HEPES, pH 7.4, 1 mM Mg₂SO₄, 0.03% H₂O₂, 2.5 mM DAB | Clear | Light Brown | 1+ ppt, slight brown |
| 35 | 100 mM MES, pH 6.5, Nothing, 0.03% H₂O₂, 2.5 mM DAB | Clear | Clear | 2+ ppt, slight brown |
| 36 | 100 mM MES, pH 6.5, 50 mM Polyethylene Glycol, 0.03% H₂O₂, 2.5 mM DAB | Clear | Clear | 0 ppt, slight brown |
| 37 | 100 mM MES, pH 6.5, 50 mM Diethylene Glycol, 0.03% H₂O₂, 2.5 mM DAB | Clear | Clear | 2+ ppt, slight brown |
| 38 | 100 mM MES, pH 6.5, 50 mM Propanediol, 0.03% H₂O₂, 2.5 mM DAB | Clear | Clear | 2+ ppt, slight brown |
| 39 | 100 mM MES, pH 6.5, 50 mM Glycerol, 0.03% H₂O₂, 2.5 mM DAB | Clear | Clear | 2+ ppt, slight brown |
| 40 | 100 mM MES, pH 6.5, 50 mM Ribose, 0.03% H₂O₂, 2.5 mM DAB | Clear | Clear | No ppt, clear |
| 41 | 100 mM MES, pH 6.5, 1 mM EDTA, 0.03% H₂O₂, 2.5 mM DAB | Slight Brown | Slight Brown | 2+ ppt, slight brown |
| 42 | 100 mM MES, pH 6.5, 1 mM DTPA, 0.03% H₂O₂, 2.5 mM DAB | Clear | Clear | 1+ ppt, slight brown |
| 43 | 100 mM MES, pH 6.5, 1 mM EGTA, 0.03% H₂O₂, 2.5 mM DAB | Dark Brown | Brown, Murky | 3+ ppt, brown |
| 44 | 100 mM MES, pH 6.5, 1 mM 1,10-Phenanthroline, 0.03% H₂O₂, 2.5 mM DAB | Clear | Clear | 1+ ppt, slight brown |
| 45 | 100 mM MES, pH 6.5, 1 mM Diethylenetriamine.., 0.03% H₂O₂, 2.5 mM DAB | Clear | Clear | 0 ppt, clear |
| 46 | 100 mM MES, pH 6.5, 10 mM Sodium Metabisulfite, 0.03% H₂O₂, 2.5 mM DAB | Clear | Clear | 0 ppt, clear |
| 47 | 100 mM MES, pH 6.5, 10 mM Ascorbic Acid, 0.03% H₂O₂, 2.5 mM DAB | Yellow | Dark Yellow | 2+ ppt, yellow |
| 48 | 100 mM MES, pH 6.5, 10 mM Acetonitrile, 0.03% H₂O₂, 2.5 mM DAB | | Clear | 2+ ppt, slight brown |
| 49 | 100 mM MES, pH 6.5, 1 mM CuCl₂, 0.03% H₂O₂, 2.5 mM DAB | Black with | Black, Ppt | 3+ ppt, black |
| | | Large Ppt | | |
| 50 | 100 mM MES, pH 6.5, 1 mM CoCl₂, 0.03% H₂O₂, 2.5 mM DAB | Black, | Black | 4+ ppt, black |
| | | No Ppt | | |
| 51 | 100 mM MES, pH 6.5, 1 mM Mg₂SO₄, 0.03% H₂O₂, 2.5 mM DAB | Clear | Clear | 1+ ppt, slight brown |
| 52 | 100 mM Imidazole, Nothing, 0.03% H₂O₂, 2.5 mM DAB | Light Brown | Light Brown | 2+ ppt, slight brown |
| 53 | 100 mM Imidazole, 50 mM Polyethylene Glycol, 0.03% H₂O₂, 2.5 mM DAB | Light Brown | Light Brown | 0 ppt, medium brown |
| 54 | 100 mM Imidazole, 50 mM Diethylene Glycol, 0.03% H₂O₂, 2.5 mM DAB | Light Brown | Light Brown | 2+ ppt, slight brown |
| 55 | 100 mM Imidazole, 50 mM Propanediol, 0.03% H₂O₂, 2.5 mM DAB | Light Brown | Light Brown | 1+ ppt, slight brown |
| 56 | 100 mM Imidazole, 50 mM Glycerol, 0.03% H₂O₂, 2.5 mM DAB | Light Brown | Light Brown | 2+ ppt, slight brown |
| 57 | 100 mM Imidazole, 50 mM Ribose, 0.03% H₂O₂, 2.5 mM DAB | Light Brown | Light Brown | 2+ ppt, slight brown |
| 58 | 100 mM Imidazole, 1 mM EDTA, 0.03% H₂O₂, 2.5 mM DAB | Dark Brown | Murky Brown | 3+ ppt, medium brown |
| 59 | 100 mM Imidazole, 1 mM DTPA, 0.03% H₂O₂, 2.5 mM DAB | Light Brown | Light Brown | 2+ ppt, medium brown |
| 60 | 100 mM Imidazole, 1 mM EGTA, 0.03% H₂O₂, 2.5 mM DAB | Light, murky | Darker Brown | 4+ ppt, medium brown |
| | | Brown | | |
| 61 | 100 mM Imidazole, 1 mM 1,10-Phenanthroline, 0.03% H₂O₂, 2.5 mM DAB | Light Brown | Clear | 2+ ppt, medium brown |
| 62 | 100 mM Imidazole, 1 mM Diethylenetriamine.., 0.03% H₂O₂, 2.5 mM DAB | Light Brown | Clear | 0.5 ppt, slight brown |
| 63 | 100 mM Imidazole, 10 mM Sodium Metabisulfite, 0.03% H₂O₂, 2.5 mM DAB | Light Brown | Clear | 0 ppt, clear |
| 64 | 100 mM Imidazole, 10 mM Ascorbic Acid, 0.03% H₂O₂, 2.5 mM DAB | Dark Orange | Darker | 2+ ppt, yellow |
| | | | Yellow | |
| 65 | 100 mM Imidazole, 10 mM Acetonitrile, 0.03% H₂O₂, 2.5 mM DAB | Light Brown | Light Brown | 2+ ppt, slight brown |
| 66 | 100 mM Imidazole, 1 mM CuCl₂, 0.03% H₂O₂, 2.5 mM DAB | Black, | Black | 3+ ppt, black |
| | | No Ppt | | |
| 67 | 100 mM Imidazole, 1 mM CoCl₂, 0.03% H₂O₂, 2.5 mM DAB | Black, | Black | 4+ ppt, black |
| | | No Ppt | | |
| 68 | 100 mM Imidazole, 1 mM Mg₂SO₄, 0.03% H₂O₂, 2.5 mM DAB | Very Light | Light Brown | 2+ ppt, slight brown |
| | | Brown | | |
| 69 | Control | Medium Brown | Dark Brown | 3+ ppt, medium brown |

Buffer, hydrogen peroxide and DAB were mixed and the color was noted after incubation at 37° C. for the indicated period of time. “Ppt” refers to the formation of a precipitate.

TABLE 8
Evaluation of DAB Absorbance spectra in different buffers. The indicated components were mixed and the absorbance spectra
determined using a NanoDrop ND-1000 spectrophotometer. The absorbance at 280 nm was recorded following incubation at 37° C. for the
indicated period of times. After 16 hours, 1 μL of Anti-Mouse HRP Polymer Conjugate (Cat. No. 84-0146) was added, the reactions were
incubated at 37° C. for 1 hour and the spectrum again measured. The loss of signal with time indicates the precipitation of DAB with the
corresponding loss of dye in solution.

| | | Abs 280 nm at | | | | | Ratio Signal Time T/ | | | |
|---|---|---|---|---|---|---|---|---|---|---|
| | | indicated time | | | | | Signal Time 0 | | | |
| | | | | | | After | | | | After |
| | Abs 280 nm | 0 | 2 Hr | 4 Hr | 16 Hr | Enz | 2 hr | 4 hr | 16 Hr | Enz |
| 1 | 100 mM Tris, pH 8, Nothing, 0.03% H₂O₂, 2.5 mM DAB | 3.43 | 3.36 | 3.19 | 2.94 | 2.83 | 1.0 | 0.9 | 0.9 | 0.8 |
| 2 | 100 mM Tris, pH 8, 50 mM Polyethylene Glycol, 0.03% H₂O₂, 2.5 mM DAB | 3.61 | 3.65 | 3.52 | 3.34 | 2.96 | 1.0 | 1.0 | 0.9 | 0.8 |
| 3 | 100 mM Tris, pH 8, 50 mM Diethylene Glycol, 0.03% H₂O₂, 2.5 mM DAB | 3.52 | 3.49 | 3.43 | 3.11 | 3.01 | 1.0 | 1.0 | 0.9 | 0.9 |
| 4 | 100 mM Tris, pH 8, 50 mM Propanediol, 0.03% H₂O₂, 2.5 mM DAB | 3.64 | 3.55 | 3.48 | 3.24 | 3.04 | 1.0 | 1.0 | 0.9 | 0.8 |
| 5 | 100 mM Tris, pH 8, 50 mM Glycerol, 0.03% H₂O₂, 2.5 mM DAB | 3.52 | 3.49 | 3.44 | 3.2 | 3.04 | 1.0 | 1.0 | 0.9 | 0.9 |
| 6 | 100 mM Tris, pH 8, 50 mM Ribose, 0.03% H₂O₂, 2.5 mM DAB | 3.27 | 3.56 | 3.52 | 3.41 | 3.3 | 1.1 | 1.1 | 1.0 | 1.0 |
| 7 | 100 mM Tris, pH 8, 1 mM EDTA, 0.03% H₂O₂, 2.5 mM DAB | 3.45 | 3.48 | 3.3 | 2.66 | 2.44 | 1.0 | 1.0 | 0.8 | 0.7 |
| 8 | 100 mM Tris, pH 8, 1 mM DTPA, 0.03% H₂O₂, 2.5 mM DAB | 3.27 | 3.61 | 3.51 | 2.89 | 2.61 | 1.1 | 1.1 | 0.9 | 0.8 |
| 9 | 100 mM Tris, pH 8, 1 mM EGTA, 0.03% H₂O₂, 2.5 mM DAB | 3.54 | 2.88 | 2.16 | 0.17 | 0.07 | 0.8 | 0.6 | 0.0 | 0.0 |
| 10 | 100 mM Tris, pH 8, 1 mM 1,10-Phenanthroline, 0.03% H₂O₂, 2.5 mM DAB | 4.88 | 4.76 | 4.84 | 4.34 | 4.09 | 1.0 | 1.0 | 0.9 | 0.8 |
| 11 | 100 mM Tris, pH 8, 1 mM Diethylenetriamine . . . , 0.03% H₂O₂, 2.5 mM DAB | 3.48 | 3.31 | 3.31 | 3.22 | 3.09 | 1.0 | 1.0 | 0.9 | 0.9 |
| 12 | 100 mM Tris, pH 8, 10 mM Sodium Metabisulfite, 0.03% H₂O₂, 2.5 mM DAB | 3.58 | 3.57 | 3.56 | 3.55 | 3.57 | 1.0 | 1.0 | 1.0 | 1.0 |
| 13 | 100 mM Tris, pH 8, 10 mM Ascorbic Acid, 0.03% H₂O₂, 2.5 mM DAB | 9.54 | 8.87 | 7.75 | 5.43 | 4.75 | 0.9 | 0.8 | 0.6 | 0.5 |
| 14 | 100 mM Tris, pH 8, 10 mM Acetonitrile, 0.03% H₂O₂, 2.5 mM DAB | 3.66 | 3.59 | 3.53 | 3.22 | 3.07 | 1.0 | 1.0 | 0.9 | 0.8 |
| 15 | 100 mM Tris, pH 8, 1 mM CuCl₂, 0.03% H₂O₂, 2.5 mM DAB | 0.33 | 0.12 | 0.17 | 0.16 | 0.21 | 0.4 | 0.5 | 0.5 | 0.6 |
| 16 | 100 mM Tris, pH 8, 1 mM CoCl₂, 0.03% H₂O₂, 2.5 mM DAB | 2.15 | 0.26 | 0.16 | 0.19 | 0.17 | 0.1 | 0.1 | 0.1 | 0.1 |
| 17 | 100 mM Tris, pH 8, 1 mM Mg₂SO₄, 0.03% H₂O₂, 2.5 mM DAB | 3.47 | 3.43 | 3.36 | 3.03 | 2.94 | 1.0 | 1.0 | 0.9 | 0.8 |
| 18 | 100 mM HEPES, pH 7.4, Nothing, 0.03% H₂O₂, 2.5 mM DAB | 3.59 | 3.63 | 3.61 | 3.36 | 3.08 | 1.0 | 1.0 | 0.9 | 0.9 |
| 19 | 100 mM HEPES, pH 7.4, 50 mM Polyethylene Glycol, 0.03% H₂O₂, | 3.21 | 3.33 | 3.36 | 3.26 | 2.72 | 1.0 | 1.0 | 1.0 | 0.8 |
| | 2.5 mM DAB | | | | | | | | | |
| 20 | 100 mM HEPES, pH 7.4, 50 mM Diethylene Glycol, 0.03% H₂O₂, 2.5 mM DAB | 3.43 | 3.47 | 3.41 | 3.22 | 2.87 | 1.0 | 1.0 | 0.9 | 0.8 |
| 21 | 100 mM HEPES, pH 7.4, 50 mM Propanediol, 0.03% H₂O₂, 2.5 mM DAB | 3.47 | 3.5 | 3.45 | 3.22 | 2.93 | 1.0 | 1.0 | 0.9 | 0.8 |
| 22 | 100 mM HEPES, pH 7.4, 50 mM Glycerol, 0.03% H₂O₂, 2.5 mM DAB | 3.41 | 3.47 | 3.46 | 3.15 | 3 | 1.0 | 1.0 | 0.9 | 0.9 |
| 23 | 100 mM HEPES, pH 7.4, 50 mM Ribose, 0.03% H₂O₂, 2.5 mM DAB | 3.49 | 3.56 | 3.5 | 3.34 | 3.29 | 1.0 | 1.0 | 1.0 | 0.9 |
| 24 | 100 mM HEPES, pH 7.4, 1 mM EDTA, 0.03% H₂O₂, 2.5 mM DAB | 3.66 | 3.57 | 3.4 | 2.7 | 2.43 | 1.0 | 0.9 | 0.7 | 0.7 |
| 25 | 100 mM HEPES, pH 7.4, 1 mM DTPA, 0.03% H₂O₂, 2.5 mM DAB | 3.51 | 3.58 | 3.52 | 3.12 | 2.84 | 1.0 | 1.0 | 0.9 | 0.8 |
| 26 | 100 mM HEPES, pH 7.4, 1 mM EGTA, 0.03% H₂O₂, 2.5 mM DAB | 3.63 | 3.05 | 2.55 | 1.42 | 1.18 | 0.8 | 0.7 | 0.4 | 0.3 |
| 27 | 100 mM HEPES, pH 7.4, 1 mM 1,10-Phenanthroline, 0.03% H₂O₂, 2.5 mM DAB | 4.93 | 0.9 | 5.13 | 4.37 | 4.06 | 0.2 | 1.0 | 0.9 | 0.8 |
| 28 | 100 mM HEPES, pH 7.4, 1 mM Diethylenetriamine . . . , 0.03% H₂O₂, | 3.6 | 3.56 | 3.54 | 3.44 | 3.19 | 1.0 | 1.0 | 1.0 | 0.9 |
| | 2.5 mM DAB | | | | | | | | | |
| 29 | 100 mM HEPES, pH 7.4, 10 mM Sodium Metabisulfite, 0.03% H₂O₂, | 3.38 | 3.45 | 3.4 | 3.44 | 3.43 | 1.0 | 1.0 | 1.0 | 1.0 |
| | 2.5 mM DAB | | | | | | | | | |
| 30 | 100 mM HEPES, pH 7.4, 10 mM Ascorbic Acid, 0.03% H₂O₂, 2.5 mM DAB | 8.61 | 7.11 | 6.43 | 4.95 | 4.66 | 0.8 | 0.7 | 0.6 | 0.5 |
| 31 | 100 mM HEPES, pH 7.4, 10 mM Acetonitrile, 0.03% H₂O₂, 2.5 mM DAB | 3.45 | 3.49 | 3.49 | 3.25 | 2.89 | 1.0 | 1.0 | 0.9 | 0.8 |
| 32 | 100 mM HEPES, pH 7.4, 1 mM CuCl₂, 0.03% H₂O₂, 2.5 mM DAB | 2.82 | 2.54 | 2.3 | 1.3 | 1.18 | 0.9 | 0.8 | 0.5 | 0.4 |
| 33 | 100 mM HEPES, pH 7.4, 1 mM CoCl₂, 0.03% H₂O₂, 2.5 mM DAB | 3.31 | 1.09 | 0.06 | 0.05 | 0.05 | 0.3 | 0.0 | 0.0 | 0.0 |
| 34 | 100 mM HEPES, pH 7.4, 1 mM Mg₂SO₄, 0.03% H₂O₂, 2.5 mM DAB | 3.58 | 3.58 | 3.53 | 3.27 | 2.92 | 1.0 | 1.0 | 0.9 | 0.8 |
| 35 | 100 mM MES, pH 6.5, Nothing, 0.03% H₂O₂, 2.5 mM DAB | 3.55 | 3.6 | 3.49 | 3.1 | 2.88 | 1.0 | 1.0 | 0.9 | 0.8 |
| 36 | 100 mM MES, pH 6.5, 50 mM Polyethylene Glycol, 0.03% H₂O₂, 2.5 mM DAB | 3.62 | 3.61 | 3.53 | 3.46 | 3.14 | 1.0 | 1.0 | 1.0 | 0.9 |
| 37 | 100 mM MES, pH 6.5, 50 mM Diethylene Glycol, 0.03% H₂O₂, 2.5 mM DAB | 3.31 | 3.35 | 3.32 | 2.98 | 2.63 | 1.0 | 1.0 | 0.9 | 0.8 |
| 38 | 100 mM MES, pH 6.5, 50 mM Propanediol, 0.03% H₂O₂, 2.5 mM DAB | 3.5 | 3.57 | 3.54 | 3.12 | 2.68 | 1.0 | 1.0 | 0.9 | 0.8 |
| 39 | 100 mM MES, pH 6.5, 50 mM Glycerol, 0.03% H₂O₂, 2.5 mM DAB | 3.55 | 3.63 | 3.55 | 3.22 | 2.79 | 1.0 | 1.0 | 0.9 | 0.8 |
| 40 | 100 mM MES, pH 6.5, 50 mM Ribose, 0.03% H₂O₂, 2.5 mM DAB | 3.66 | 3.57 | 3.44 | 3.37 | 3.46 | 1.0 | 0.9 | 0.9 | 0.9 |
| 41 | 100 mM MES, pH 6.5, 1 mM EDTA, 0.03% H₂O₂, 2.5 mM DAB | 3.35 | 3.23 | 2.99 | 2.26 | 2 | 1.0 | 0.9 | 0.7 | 0.6 |
| 42 | 100 mM MES, pH 6.5, 1 mM DTPA, 0.03% H₂O₂, 2.5 mM DAB | 3.57 | 3.65 | 3.54 | 3.34 | 3 | 1.0 | 1.0 | 0.9 | 0.8 |
| 43 | 100 mM MES, pH 6.5, 1 mM EGTA, 0.03% H₂O₂, 2.5 mM DAB | 3.33 | 2.91 | 2.54 | 2.1 | 1.76 | 0.9 | 0.8 | 0.6 | 0.5 |
| 44 | 100 mM MES, pH 6.5, 1 mM 1,10-Phenanthroline, 0.03% H₂O₂, 2.5 mM DAB | 5 | 4.99 | 4.96 | 4.55 | 4.12 | 1.0 | 1.0 | 0.9 | 0.8 |
| 45 | 100 mM MES, pH 6.5, 1 mM Diethylenetriamine . . . , 0.03% H₂O₂, | 3.44 | 3.49 | 3.42 | 3.37 | 2.94 | 1.0 | 1.0 | 1.0 | 0.9 |
| | 2.5 mM DAB | | | | | | | | | |
| 46 | 100 mM MES, pH 6.5, 10 mM Sodium Metabisulfite, 0.03% H₂O₂, | 3.62 | 3.74 | 3.59 | 3.65 | 3.58 | 1.0 | 1.0 | 1.0 | 1.0 |
| | 2.5 mM DAB | | | | | | | | | |
| 47 | 100 mM MES, pH 6.5, 10 mM Ascorbic Acid, 0.03% H₂O₂, 2.5 mM DAB | 7.79 | 5.36 | 5.1 | 4.47 | 4.27 | 0.7 | 0.7 | 0.6 | 0.5 |
| 48 | 100 mM MES, pH 6.5, 10 mM Acetonitrile, 0.03% H₂O₂, 2.5 mM DAB | 3.57 | 3.59 | 3.54 | 3.14 | 2.76 | 1.0 | 1.0 | 0.9 | 0.8 |
| 49 | 100 mM MES, pH 6.5, 1 mM CuCl₂, 0.03% H₂O₂, 2.5 mM DAB | 1.06 | 0.09 | 0.02 | 0.04 | 0.04 | 0.1 | 0.0 | 0.0 | 0.0 |
| 50 | 100 mM MES, pH 6.5, 1 mM CoCl₂, 0.03% H₂O₂, 2.5 mM DAB | 3.56 | 2.76 | 1.48 | 0.03 | 0.04 | 0.8 | 0.4 | 0.0 | 0.0 |
| 51 | 100 mM MES, pH 6.5, 1 mM Mg₂SO₄, 0.03% H₂O₂, 2.5 mM DAB | 3.62 | 3.67 | 3.53 | 3.38 | 2.77 | 1.0 | 1.0 | 0.9 | 0.8 |
| 52 | 100 mM Imidazole, Nothing, 0.03% H₂O₂, 2.5 mM DAB | 3.45 | 3.41 | 3.34 | 3 | 2.85 | 1.0 | 1.0 | 0.9 | 0.8 |
| 53 | 100 mM Imidazole, 50 mM Polyethylene Glycol, 0.03% H₂O₂, 2.5 mM DAB | 3.55 | 3.52 | 3.45 | 3.29 | 2.94 | 1.0 | 1.0 | 0.9 | 0.8 |
| 54 | 100 mM Imidazole, 50 mM Diethylene Glycol, 0.03% H₂O₂, 2.5 mM DAB | 3.65 | 3.59 | 3.48 | 3.14 | 3.03 | 1.0 | 1.0 | 0.9 | 0.8 |
| 55 | 100 mM Imidazole, 50 mM Propanediol, 0.03% H₂O₂, 2.5 mM DAB | 3.46 | 3.41 | 3.35 | 3.07 | 2.97 | 1.0 | 1.0 | 0.9 | 0.9 |
| 56 | 100 mM Imidazole, 50 mM Glycerol, 0.03% H₂O₂, 2.5 mM DAB | 3.47 | 3.42 | 3.4 | 3.16 | 3.02 | 1.0 | 1.0 | 0.9 | 0.9 |
| 57 | 100 mM Imidazole, 50 mM Ribose, 0.03% H₂O₂, 2.5 mM DAB | 3.52 | 3.45 | 3.4 | 3.28 | 3.07 | 1.0 | 1.0 | 0.9 | 0.9 |
| 58 | 100 mM Imidazole, 1 mM EDTA, 0.03% H₂O₂, 2.5 mM DAB | 3.58 | 3.07 | 2.62 | 1.02 | 0.82 | 0.9 | 0.7 | 0.3 | 0.2 |
| 59 | 100 mM Imidazole, 1 mM DTPA, 0.03% H₂O₂, 2.5 mM DAB | 3.69 | 3.67 | 3.48 | 2.33 | 2.1 | 1.0 | 0.9 | 0.6 | 0.6 |
| 60 | 100 mM Imidazole, 1 mM EGTA, 0.03% H₂O₂, 2.5 mM DAB | 3.41 | 2.06 | 1.37 | 0.16 | 0.09 | 0.6 | 0.4 | 0.0 | 0.0 |
| 61 | 100 mM Imidazole, 1 mM 1,10-Phenanthroline, 0.03% H₂O₂, 2.5 mM DAB | 5.02 | 4.93 | 4.7 | 4.11 | 3.92 | 1.0 | 0.9 | 0.8 | 0.8 |
| 62 | 100 mM Imidazole, 1 mM Diethylenetriamine . . . , 0.03% H₂O₂, 2.5 mM DAB | 3.5 | 3.55 | 3.45 | 3.35 | 3.16 | 1.0 | 1.0 | 1.0 | 0.9 |
| 63 | 100 mM Imidazole, 10 mM Sodium Metabisulfite, 0.03% H₂O₂, 2.5 mM DAB | 3.45 | 3.51 | 3.45 | 3.47 | 3.4 | 1.0 | 1.0 | 1.0 | 1.0 |
| 64 | 100 mM Imidazole, 10 mM Ascorbic Acid, 0.03% H₂O₂, 2.5 mM DAB | 8.61 | 7.19 | 6.64 | 5.19 | 4.79 | 0.8 | 0.8 | 0.6 | 0.6 |
| 65 | 100 mM Imidazole, 10 mM Acetonitrile, 0.03% H₂O₂, 2.5 mM DAB | 3.59 | 3.58 | 3.44 | 2.57 | 2.35 | 1.0 | 1.0 | 0.7 | 0.7 |
| 66 | 100 mM Imidazole, 1 mM CuCl₂, 0.03% H₂O₂, 2.5 mM DAB | 2.22 | 0.89 | 0.7 | 0.07 | 0.09 | 0.4 | 0.3 | 0.0 | 0.0 |
| 67 | 100 mM Imidazole, 1 mM CoCl₂, 0.03% H₂O₂, 2.5 mM DAB | 3.17 | 2.72 | 2.73 | 0.08 | 0.09 | 0.9 | 0.9 | 0.0 | 0.0 |
| 68 | 100 mM Imidazole, 1 mM Mg₂SO₄, 0.03% H₂O₂, 2.5 mM DAB | 3.66 | 3.57 | 3.47 | 3.25 | 2.98 | 1.0 | 0.9 | 0.9 | 0.8 |
| 69 | Control | 0 | 3.38 | 2.49 | 1.85 | 1.69 | 1.0 | 0.7 | 0.5 | 0.5 |

TABLE 9
Evaluation of DAB Absorbance spectra in different buffers. The indicated components were mixed and the absorbance spectra
determined using a NanoDrop ND-1000 spectrophotometer. The absorbance at 478 nm was recorded following incubation at 37° C. for the
indicated period of times. After 16 hours, 1 μL of Anti-Mouse HRP Polymer Conjugate (Cat. No. 84-0146) was added, the reactions were
incubated at 37° C. for 1 hour and the spectrum again measured. The absorbance at 478 nm must be interpreted in the light of 280 nm, which
indicates the loss of dye from solution.

| | | Abs 478 nm at | | | | | Ratio Signal Time T/ | | | |
|---|---|---|---|---|---|---|---|---|---|---|
| | | indicated time | | | | | Signal Time 0 | | | |
| | | | | | | After | | | | After |
| | Abs 478 nm | 0 | 2 Hr | 4 Hr | 16 Hr | Enz | 2 hr | 4 hr | 16 Hr | Enz |
| 1 | 100 mM Tris, pH 8, Nothing, 0.03% H₂O₂, 2.5 mM DAB | 0.0070 | 0.029 | 0.031 | 0.011 | 0.028 | 4.1 | 4.4 | 1.6 | 4.0 |
| 2 | 100 mM Tris, pH 8, 50 mM Polyethylene Glycol, 0.03% H₂O₂, 2.5 mM DAB | 0.0080 | 0.037 | 0.036 | 0.023 | 0.035 | 4.6 | 4.5 | 2.9 | 4.4 |
| 3 | 100 mM Tris, pH 8, 50 mM Diethylene Glycol, 0.03% H₂O₂, 2.5 mM DAB | 0.0100 | 0.023 | 0.033 | 0.012 | 0.014 | 2.3 | 3.3 | 1.2 | 1.4 |
| 4 | 100 mM Tris, pH 8, 50 mM Propanediol, 0.03% H₂O₂, 2.5 mM DAB | 0.0100 | 0.032 | 0.034 | 0.012 | 0.013 | 3.2 | 3.4 | 1.2 | 1.3 |
| 5 | 100 mM Tris, pH 8, 50 mM Glycerol, 0.03% H₂O₂, 2.5 mM DAB | 0.0110 | 0.03 | 0.034 | 0.012 | 0.017 | 2.7 | 3.1 | 1.1 | 1.5 |
| 6 | 100 mM Tris, pH 8, 50 mM Ribose, 0.03% H₂O₂, 2.5 mM DAB | 0.0090 | 0.017 | 0.027 | 0.015 | 0.016 | 1.9 | 3.0 | 1.7 | 1.8 |
| 7 | 100 mM Tris, pH 8, 1 mM EDTA, 0.03% H₂O₂, 2.5 mM DAB | 0.0120 | 0.036 | 0.045 | 0.012 | 0.011 | 3.0 | 3.8 | 1.0 | 0.9 |
| 8 | 100 mM Tris, pH 8, 1 mM DTPA, 0.03% H₂O₂, 2.5 mM DAB | 0.0080 | 0.012 | 0.024 | 0.015 | 0.013 | 1.5 | 3.0 | 1.9 | 1.6 |
| 9 | 100 mM Tris, pH 8, 1 mM EGTA, 0.03% H₂O₂, 2.5 mM DAB | 0.0170 | 0.121 | 0.119 | 0.016 | 0.005 | 7.1 | 7.0 | 0.9 | 0.3 |
| 10 | 100 mM Tris, pH 8, 1 mM 1,10-Phenanthroline, 0.03% H₂O₂, 2.5 mM DAB | 0.0100 | 0.023 | 0.034 | 0.014 | 0.011 | 2.3 | 3.4 | 1.4 | 1.1 |
| 11 | 100 mM Tris, pH 8, 1 mM Diethylenetriamine . . . , 0.03% H₂O₂, | 0.0090 | 0.014 | 0.013 | 0.013 | 0.017 | 1.6 | 1.4 | 1.4 | 1.9 |
| | 2.5 mM DAB | | | | | | | | | |
| 12 | 100 mM Tris, pH 8, 10 mM Sodium Metabisulfite, 0.03% H₂O₂, 2.5 mM DAB | 0.0100 | 0.008 | 0.005 | 0.008 | 0.005 | 0.8 | 0.5 | 0.8 | 0.5 |
| 13 | 100 mM Tris, pH 8, 10 mM Ascorbic Acid, 0.03% H₂O₂, 2.5 mM DAB | 0.0190 | 0.087 | 0.107 | 0.123 | 0.132 | 4.6 | 5.6 | 6.5 | 6.9 |
| 14 | 100 mM Tris, pH 8, 10 mM Acetonitrile, 0.03% H₂O₂, 2.5 mM DAB | 0.0120 | 0.038 | 0.04 | 0.012 | 0.014 | 3.2 | 3.3 | 1.0 | 1.2 |
| 15 | 100 mM Tris, pH 8, 1 mM CuCl₂, 0.03% H₂O₂, 2.5 mM DAB | 0.0010 | 0.018 | 0.02 | 0.006 | 0.006 | 18.0 | 20.0 | 6.0 | 6.0 |
| 16 | 100 mM Tris, pH 8, 1 mM CoCl₂, 0.03% H₂O₂, 2.5 mM DAB | 0.0870 | 0.012 | 0.007 | 0.017 | 0.019 | 0.1 | 0.1 | 0.2 | 0.2 |
| 17 | 100 mM Tris, pH 8, 1 mM Mg₂SO₄, 0.03% H₂O₂, 2.5 mM DAB | 0.0140 | 0.028 | 0.034 | 0.014 | 0.014 | 2.0 | 2.4 | 1.0 | 1.0 |
| 18 | 100 mM HEPES, pH 7.4, Nothing, 0.03% H₂O₂, 2.5 mM DAB | 0.0070 | 0.012 | 0.017 | 0.014 | 0.017 | 1.7 | 2.4 | 2.0 | 2.4 |
| 19 | 100 mM HEPES, pH 7.4, 50 mM Polyethylene Glycol, 0.03% H₂O₂, | 0.0120 | 0.015 | 0.017 | 0.043 | 0.04 | 1.3 | 1.4 | 3.6 | 3.3 |
| | 2.5 mM DAB | | | | | | | | | |
| 20 | 100 mM HEPES, pH 7.4, 50 mM Diethylene Glycol, 0.03% H₂O₂, | 0.0090 | 0.01 | 0.016 | 0.014 | 0.016 | 1.1 | 1.8 | 1.6 | 1.8 |
| | 2.5 mM DAB | | | | | | | | | |
| 21 | 100 mM HEPES, pH 7.4, 50 mM Propanediol, 0.03% H₂O₂, 2.5 mM DAB | 0.0070 | 0.012 | 0.016 | 0.014 | 0.01 | 1.7 | 2.3 | 2.0 | 1.4 |
| 22 | 100 mM HEPES, pH 7.4, 50 mM Glycerol, 0.03% H₂O₂, 2.5 mM DAB | 0.0110 | 0.012 | 0.015 | 0.013 | 0.016 | 1.1 | 1.4 | 1.2 | 1.5 |
| 23 | 100 mM HEPES, pH 7.4, 50 mM Ribose, 0.03% H₂O₂, 2.5 mM DAB | 0.0090 | 0.009 | 0.009 | 0.012 | 0.015 | 1.0 | 1.0 | 1.3 | 1.7 |
| 24 | 100 mM HEPES, pH 7.4, 1 mM EDTA, 0.03% H₂O₂, 2.5 mM DAB | 0.0100 | 0.037 | 0.035 | 0.016 | 0.019 | 3.7 | 3.5 | 1.6 | 1.9 |
| 25 | 100 mM HEPES, pH 7.4, 1 mM DTPA, 0.03% H₂O₂, 2.5 mM DAB | 0.0080 | 0.014 | 0.021 | 0.016 | 0.019 | 1.8 | 2.6 | 2.0 | 2.4 |
| 26 | 100 mM HEPES, pH 7.4, 1 mM EGTA, 0.03% H₂O₂, 2.5 mM DAB | 0.0160 | 0.061 | 0.036 | 0.022 | 0.014 | 3.8 | 2.3 | 1.4 | 0.9 |
| 27 | 100 mM HEPES, pH 7.4, 1 mM 1,10-Phenanthroline, 0.03% H₂O₂, | 0.0100 | 0.135 | 0.032 | 0.016 | 0.018 | 13.5 | 3.2 | 1.6 | 1.8 |
| | 2.5 mM DAB | | | | | | | | | |
| 28 | 100 mM HEPES, pH 7.4, 1 mM Diethylenetriamine . . . , 0.03% H₂O₂, | 0.0090 | 0.01 | 0.014 | 0.023 | 0.015 | 1.1 | 1.6 | 2.6 | 1.7 |
| | 2.5 mM DAB | | | | | | | | | |
| 29 | 100 mM HEPES, pH 7.4, 10 mM Sodium Metabisulfite, 0.03% H₂O₂, | 0.0080 | 0.008 | 0.008 | 0.01 | 0.013 | 1.0 | 1.0 | 1.3 | 1.6 |
| | 2.5 mM DAB | | | | | | | | | |
| 30 | 100 mM HEPES, pH 7.4, 10 mM Ascorbic Acid, 0.03% H₂O₂, 2.5 mM DAB | 0.1670 | 0.15 | 0.167 | 0.094 | 0.068 | 0.9 | 1.0 | 0.6 | 0.4 |
| 31 | 100 mM HEPES, pH 7.4, 10 mM Acetonitrile, 0.03% H₂O₂, 2.5 mM DAB | 0.0100 | 0.013 | 0.018 | 0.016 | 0.018 | 1.3 | 1.8 | 1.6 | 1.8 |
| 32 | 100 mM HEPES, pH 7.4, 1 mM CuCl₂, 0.03% H₂O₂, 2.5 mM DAB | 0.3270 | 0.358 | 0.391 | 0.035 | 0.024 | 1.1 | 1.2 | 0.1 | 0.1 |
| 33 | 100 mM HEPES, pH 7.4, 1 mM CoCl₂, 0.03% H₂O₂, 2.5 mM DAB | 0.0410 | 0.108 | 0.031 | 0.006 | 0.01 | 2.6 | 0.8 | 0.1 | 0.2 |
| 34 | 100 mM HEPES, pH 7.4, 1 mM Mg₂SO₄, 0.03% H₂O₂, 2.5 mM DAB | 0.0100 | 0.013 | 0.016 | 0.016 | 0.017 | 1.3 | 1.6 | 1.6 | 1.7 |
| 35 | 100 mM MES, pH 6.5, Nothing, 0.03% H₂O₂, 2.5 mM DAB | 0.0090 | 0.014 | 0.016 | 0.014 | 0.014 | 1.6 | 1.8 | 1.6 | 1.6 |
| 36 | 100 mM MES, pH 6.5, 50 mM Polyethylene Glycol, 0.03% H₂O₂, | 0.0100 | 0.014 | 0.017 | 0.037 | 0.028 | 1.4 | 1.7 | 3.7 | 2.8 |
| | 2.5 mM DAB | | | | | | | | | |
| 37 | 100 mM MES, pH 6.5, 50 mM Diethylene Glycol, 0.03% H₂O₂, 2.5 mM DAB | 0.0080 | 0.011 | 0.014 | 0.014 | 0.015 | 1.4 | 1.8 | 1.8 | 1.9 |
| 38 | 100 mM MES, pH 6.5, 50 mM Propanediol, 0.03% H₂O₂, 2.5 mM DAB | 0.0090 | 0.014 | 0.015 | 0.018 | 0.018 | 1.6 | 1.7 | 2.0 | 2.0 |
| 39 | 100 mM MES, pH 6.5, 50 mM Glycerol, 0.03% H₂O₂, 2.5 mM DAB | 0.0080 | 0.013 | 0.018 | 0.015 | 0.029 | 1.6 | 2.3 | 1.9 | 3.6 |
| 40 | 100 mM MES, pH 6.5, 50 mM Ribose, 0.03% H₂O₂, 2.5 mM DAB | 0.0060 | 0.008 | 0.009 | 0.016 | 0.013 | 1.3 | 1.5 | 2.7 | 2.2 |
| 41 | 100 mM MES, pH 6.5, 1 mM EDTA, 0.03% H₂O₂, 2.5 mM DAB | 0.0130 | 0.034 | 0.036 | 0.017 | 0.019 | 2.6 | 2.8 | 1.3 | 1.5 |
| 42 | 100 mM MES, pH 6.5, 1 mM DTPA, 0.03% H₂O₂, 2.5 mM DAB | 0.0090 | 0.014 | 0.011 | 0.016 | 0.019 | 1.6 | 1.2 | 1.8 | 2.1 |
| 43 | 100 mM MES, pH 6.5, 1 mM EGTA, 0.03% H₂O₂, 2.5 mM DAB | 0.0260 | 0.046 | 0.022 | 0.019 | 0.018 | 1.8 | 0.8 | 0.7 | 0.7 |
| 44 | 100 mM MES, pH 6.5, 1 mM 1,10-Phenanthroline, 0.03% H₂O₂, 2.5 mM DAB | 0.0090 | 0.013 | 0.013 | 0.015 | 0.018 | 1.4 | 1.4 | 1.7 | 2.0 |
| 45 | 100 mM MES, pH 6.5, 1 mM Diethylenetriamine . . , , 0.03% H₂O₂, | 0.0060 | 0.011 | 0.008 | 0.019 | 0.016 | 1.8 | 1.3 | 3.2 | 2.7 |
| | 2.5 mM DAB | | | | | | | | | |
| 46 | 100 mM MES, pH 6.5, 10 mM Sodium Metabisulfite, 0.03% H₂O₂, | 0.0060 | 0.008 | 0.007 | 0.011 | 0.008 | 1.3 | 1.2 | 1.8 | 1.3 |
| | 2.5 mM DAB | | | | | | | | | |
| 47 | 100 mM MES, pH 6.5, 10 mM Ascorbic Acid, 0.03% H₂O₂, 2.5 mM DAB | 0.3480 | 0.249 | 0.151 | 0.074 | 0.072 | 0.7 | 0.4 | 0.2 | 0.2 |
| 48 | 100 mM MES, pH 6.5, 10 mM Acetonitrile, 0.03% H₂O₂, 2.5 mM DAB | 0.0080 | 0.015 | 0.017 | 0.016 | 0.02 | 1.9 | 2.1 | 2.0 | 2.5 |
| 49 | 100 mM MES, pH 6.5, 1 mM CuCl₂, 0.03% H₂O₂, 2.5 mM DAB | 0.0040 | 0.008 | 0.002 | 0.007 | 0.005 | 2.0 | 0.5 | 1.8 | 1.3 |
| 50 | 100 mM MES, pH 6.5, 1 mM CoCl₂, 0.03% H₂O₂, 2.5 mM DAB | 0.0300 | 0.017 | 0.009 | 0.008 | 0.007 | 0.6 | 0.3 | 0.3 | 0.2 |
| 51 | 100 mM MES, pH 6.5, 1 mM Mg₂SO₄, 0.03% H₂O₂, 2.5 mM DAB | 0.0090 | 0.016 | 0.012 | 0.016 | 0.022 | 1.8 | 1.3 | 1.8 | 2.4 |
| 52 | 100 mM Imidazole, Nothing, 0.03% H₂O₂, 2.5 mM DAB | 0.0200 | 0.043 | 0.04 | 0.014 | 0.013 | 2.2 | 2.0 | 0.7 | 0.7 |
| 53 | 100 mM Imidazole, 50 mM Polyethylene Glycol, 0.03% H₂O₂, 2.5 mM DAB | 0.0350 | 0.057 | 0.067 | 0.125 | 0.048 | 1.6 | 1.9 | 3.6 | 1.4 |
| 54 | 100 mM Imidazole, 50 mM Diethylene Glycol, 0.03% H₂O₂, 2.5 mM DAB | 0.0180 | 0.004 | 0.032 | 0.012 | 0.015 | 0.2 | 1.8 | 0.7 | 0.8 |
| 55 | 100 mM Imidazole, 50 mM Propanediol, 0.03% H₂O₂, 2.5 mM DAB | 0.0200 | 0.039 | 0.045 | 0.014 | 0.015 | 2.0 | 2.3 | 0.7 | 0.8 |
| 56 | 100 mM Imidazole, 50 mM Glycerol, 0.03% H₂O₂, 2.5 mM DAB | 0.0170 | 0.05 | 0.041 | 0.009 | 0.012 | 2.9 | 2.4 | 0.5 | 0.7 |
| 57 | 100 mM Imidazole, 50 mM Ribose, 0.03% H₂O₂, 2.5 mM DAB | 0.0150 | 0.032 | 0.033 | 0.018 | 0.02 | 2.1 | 2.2 | 1.2 | 1.3 |
| 58 | 100 mM Imidazole, 1 mM EDTA, 0.03% H₂O₂, 2.5 mM DAB | 0.0150 | 0.066 | 0.064 | 0.022 | 0.012 | 4.4 | 4.3 | 1.5 | 0.8 |
| 59 | 100 mM Imidazole, 1 mM DTPA, 0.03% H₂O₂, 2.5 mM DAB | 0.0090 | 0.035 | 0.039 | 0.021 | 0.019 | 3.9 | 4.3 | 2.3 | 2.1 |
| 60 | 100 mM Imidazole, 1 mM EGTA, 0.03% H₂O₂, 2.5 mM DAB | 0.0650 | 0.106 | 0.09 | 0.019 | 0.013 | 1.6 | 1.4 | 0.3 | 0.2 |
| 61 | 100 mM Imidazole, 1 mM 1,10-Phenanthroline, 0.03% H₂O₂, 2.5 mM DAB | 0.0090 | 0.033 | 0.041 | 0.015 | 0.02 | 3.7 | 4.6 | 1.7 | 2.2 |
| 62 | 100 mM Imidazole, 1 mM Diethylenetriamine . . . , 0.03% H₂O₂, | 0.0090 | 0.017 | 0.019 | 0.019 | 0.016 | 1.9 | 2.1 | 2.1 | 1.8 |
| | 2.5 mM DAB | | | | | | | | | |
| 63 | 100 mM Imidazole, 10 mM Sodium Metabisulfite, 0.03% H₂O₂, 2.5 mM DAB | 0.0060 | 0.01 | 0.007 | 0.013 | 0.016 | 1.7 | 1.2 | 2.2 | 2.7 |
| 64 | 100 mM Imidazole, 10 mM Ascorbic Acid, 0.03% H₂O₂, 2.5 mM DAB | 0.0960 | 0.212 | 0.162 | 0.199 | 0.148 | 2.2 | 1.7 | 2.1 | 1.5 |
| 65 | 100 mM Imidazole, 10 mM Acetonitrile, 0.03% H₂O₂, 2.5 mM DAB | 0.0060 | 0.035 | 0.04 | 0.01 | 0.021 | 5.8 | 6.7 | 1.7 | 3.5 |
| 66 | 100 mM Imidazole, 1 mM CuCl₂, 0.03% H₂O₂, 2.5 mM DAB | 0.6240 | 0.459 | 0.443 | 0.008 | 0.007 | 0.7 | 0.7 | 0.0 | 0.0 |
| 67 | 100 mM Imidazole, 1 mM CoCl₂, 0.03% H₂O₂, 2.5 mM DAB | 0.1280 | 0.47 | 0.549 | 0.071 | 0.011 | 3.7 | 4.3 | 0.6 | 0.1 |
| 68 | 100 mM Imidazole, 1 mM Mg₂SO₄, 0.03% H₂O₂, 2.5 mM DAB | 0.0220 | 0.043 | 0.049 | 0.014 | 0.017 | 2.0 | 2.2 | 0.6 | 0.8 |
| 69 | Control | | 0.144 | 0.267 | 0.157 | 0.053 | 1.0 | 1.9 | 1.1 | 0.4 |

TABLE 10
Stability of DAB-containing solutions. DAB-containing solutions were incubated at 37° C., aliquots diluted 200-fold and the
absorbances at 280 nm were measured at 0 hours, 2.5 hours and 5 hours. After 18.5 hours of incubation at 37° C., H₂O₂
was added to a final concentration of 0.03% and the absorbance at 280 nm measured (“H₂O₂”). Each DAB-containing
solution was diluted 200-fold in a solution containing 100 mM imidazole, 0.03% H₂O₂ and 1/20^{th} dilution of HRP polymer
(Solution H, Cat. No. 84-0146). The reactions were incubated an additional 1 hour at 37° C. and the absorbance at
280 nm measured (“Enzyme”).

| | | Absorbance 280 nm | | | | | |
|---|---|---|---|---|---|---|---|
| | | 0 | 2.5 | 5 | 18.5 | H₂O₂ | Enzyme |
| 1 | Water, Nothing, 50 mM DAB | 648 | 806 | 744 | 706 | 624 | 42 |
| 2 | Water, 55 mM Propylene Glycol, 56 mM DAB | 924 | 964 | 850 | 938 | 808 | 116 |
| 3 | Water, 55 mM Propanediol, 56 mM DAB | 966 | 934 | 888 | 938 | 808 | 132 |
| 4 | Water, 55 mM Diethylene Glycol, 56 mM DAB | 772 | 930 | 864 | 914 | 792 | 114 |
| 5 | Water, 55 mM Glycerol, 56 mM DAB | 1050 | 950 | 864 | 772 | 744 | 144 |
| 6 | Water, 55 mM Ribose, 56 mM DAB | 830 | 888 | 824 | 890 | 636 | 262 |
| 7 | Water, 1 mM EDTA, 50 mM DAB | 748 | 812 | 822 | 834 | 664 | 70 |
| 8 | Water, 1 mM DTPA, 50 mM DAB | 798 | 832 | 804 | 832 | 730 | 156 |
| 9 | Water, 1 mM EGTA, 50 mM DAB | 802 | 790 | 798 | 816 | 810 | 214 |
| 10 | Water, 1 mM 1,10 Phenanthroline, 50 mM DAB | 816 | 766 | 810 | 822 | 782 | 94 |
| 11 | Water, 1 mM Diethylenetriaminepentamethylene . . . , 50 mM DAB | 738 | 876 | 806 | 820 | 728 | 102 |
| 12 | Water, 10 mM Acetone, 51 mM DAB | 702 | 834 | 862 | 842 | 720 | 96 |
| 13 | Water, 10 mM Ascorbic Acid, 51 mM DAB | 872 | 842 | 1378 | 814 | 842 | 400 |
| 14 | Water, 1 mM MgCl2, 50 mM DAB | 752 | 808 | 912 | 806 | 714 | 66 |
| 15 | 100 mM Citrate, pH 3.0, Nothing, 50 mM DAB | 848 | 1630 | 638 | 662 | 612 | 30 |
| 16 | 111 mM Citrate, pH 3.0, 55 mM Propylene Glycol, 56 mM DAB | 704 | 1082 | 814 | 940 | 708 | 34 |
| 17 | 111 mM Citrate, pH 3.0, 55 mM Propanediol, 56 mM DAB | 1000 | 892 | 768 | 926 | 734 | 82 |
| 18 | 111 mM Citrate, pH 3.0, 55 mM Diethylene Glycol, 56 mM DAB | 856 | 890 | 794 | 728 | 680 | 76 |
| 19 | 111 mM Citrate, pH 3.0, 55 mM Glycerol, 56 mM DAB | 1074 | 904 | 810 | 846 | 730 | 70 |
| 20 | 111 mM Citrate, pH 3.0, 55 mM Ribose, 56 mM DAB | 734 | 804 | 732 | 900 | 608 | 192 |
| 21 | 100 mM Citrate, pH 3.0, 1 mM EDTA, 50 mM DAB | 710 | 776 | 748 | 748 | 602 | 58 |
| 22 | 100 mM Citrate, pH 3.0, 1 mM DTPA, 50 mM DAB | 660 | 850 | 740 | 744 | 726 | 38 |
| 23 | 100 mM Citrate, pH 3.0, 1 mM EGTA, 50 mM DAB | 732 | 816 | 754 | 696 | 696 | 84 |
| 24 | 100 mM Citrate, pH 3.0, 1 mM 1,10 Phenanthroline, 50 mM DAB | 802 | 884 | 758 | 752 | 728 | 68 |
| 25 | 100 mM Citrate, pH 3.0, 1 mM Diethylenetriaminepentamethylene . . . , | 666 | 722 | 692 | 828 | 714 | 38 |
| | 50 mM DAB | | | | | | |
| 26 | 102 mM Citrate, pH 3.0, 10 mM Acetone, 51 mM DAB | 752 | 786 | 600 | 802 | 644 | 114 |
| 27 | 102 mM Citrate, pH 3.0, 10 mM Ascorbic Acid, 51 mM DAB | 746 | 856 | 1160 | 750 | 764 | 142 |
| 28 | 100 mM Citrate, pH 3.0, 1 mM MgCl2, 50 mM DAB | 676 | 742 | 1046 | 712 | 640 | 32 |
| 29 | 100 mM Acetate, pH 4.0, Nothing, 50 mM DAB | 742 | 664 | 638 | 652 | 672 | 32 |
| 30 | 111 mM Acetate, pH 4.0, 55 mM Propylene Glycol, 56 mM DAB | 794 | 974 | 728 | 1144 | 768 | 122 |
| 31 | 111 mM Acetate, pH 4.0, 55 mM Propanediol, 56 mM DAB | 926 | 818 | 846 | 806 | 670 | 76 |
| 32 | 111 mM Acetate, pH 4.0, 55 mM Diethylene Glycol, 56 mM DAB | 1318 | 762 | 764 | 744 | 662 | 128 |
| 33 | 111 mM Acetate, pH 4.0, 55 mM Glycerol, 56 mM DAB | 1088 | 812 | 830 | 782 | 688 | 222 |
| 34 | 111 mM Acetate, pH 4.0, 55 mM Ribose, 56 mM DAB | 804 | 804 | 766 | 806 | 600 | 240 |
| 35 | 100 mM Acetate, pH 4.0, 1 mM EDTA, 50 mM DAB | 762 | 800 | 754 | 740 | 578 | 50 |
| 36 | 100 mM Acetate, pH 4.0, 1 mM DTPA, 50 mM DAB | 726 | 742 | 728 | 752 | 776 | 128 |
| 37 | 100 mM Acetate, pH 4.0, 1 mM EGTA, 50 mM DAB | 778 | 756 | 738 | 800 | 604 | 84 |
| 38 | 100 mM Acetate, pH 4.0, 1 mM 1,10 Phenanthroline, 50 mM DAB | 664 | 760 | 744 | 782 | 716 | 162 |
| 39 | 100 mM Acetate, pH 4.0, 1 mM Diethylenetriaminepentamethylene . . . , | 734 | 718 | 736 | 746 | 714 | 76 |
| | 50 mM DAB | | | | | | |
| 40 | 102 mM Acetate, pH 4.0, 10 mM Acetone, 51 mM DAB | 776 | 662 | 730 | 748 | 632 | 70 |
| 41 | 102 mM Acetate, pH 4.0, 10 mM Ascorbic Acid, 51 mM DAB | 760 | 748 | 1148 | 756 | 766 | 442 |
| 42 | 100 mM Acetate, pH 4.0, 1 mM MgCl2, 50 mM DAB | 632 | 744 | 908 | 760 | 640 | 276 |
| 43 | 100 mM Citrate, pH 5.0, Nothing, 50 mM DAB | 798 | 512 | 208 | 266 | 218 | 174 |
| 44 | 111 mM Citrate, pH 5.0, 55 mM Propylene Glycol, 56 mM DAB | 422 | 712 | 398 | 430 | 416 | 174 |
| 45 | 111 mM Citrate, pH 5.0, 55 mM Propanediol, 56 mM DAB | 1086 | 604 | 744 | 582 | 318 | 258 |
| 46 | 111 mM Citrate, pH 5.0, 55 mM Diethylene Glycol, 56 mM DAB | 1024 | 868 | 588 | 520 | 284 | 234 |
| 47 | 111 mM Citrate, pH 5.0, 55 mM Glycerol, 56 mM DAB | 1054 | 1194 | 856 | 386 | 298 | 200 |
| 48 | 111 mM Citrate, pH 5.0, 55 mM Ribose, 56 mM DAB | 766 | 1120 | 762 | 1072 | 626 | 398 |
| 49 | 100 mM Citrate, pH 5.0, 1 mM EDTA, 50 mM DAB | 1106 | 854 | 752 | 710 | 332 | 152 |
| 50 | 100 mM Citrate, pH 5.0, 1 mM DTPA, 50 mM DAB | 1100 | 316 | 114 | 236 | 90 | 40 |
| 51 | 100 mM Citrate, pH 5.0, 1 mM EGTA, 50 mM DAB | 1066 | 398 | 342 | 354 | 304 | 254 |
| 52 | 100 mM Citrate, pH 5.0, 1 mM 1,10 Phenanthroline, 50 mM DAB | 862 | 990 | 562 | 556 | 266 | 202 |
| 53 | 100 mM Citrate, pH 5.0, 1 mM Diethylenetriaminepentamethylene . . . , | 768 | 392 | 290 | 338 | 298 | 162 |
| | 50 mM DAB | | | | | | |
| 54 | 102 mM Citrate, pH 5.0, 10 mM Acetone, 51 mM DAB | 1142 | 338 | 466 | 366 | 468 | 172 |
| 55 | 102 mM Citrate, pH 5.0, 10 mM Ascorbic Acid, 51 mM DAB | 544 | 1068 | 518 | 336 | 324 | 370 |
| 56 | 100 mM Citrate, pH 5.0, 1 mM MgCl2, 50 mM DAB | 758 | 388 | 604 | 278 | 326 | 254 |
| 57 | 100 mM MES, pH 6.5, Nothing, 50 mM DAB | 1034 | 868 | 622 | 672 | 610 | 66 |
| 58 | 111 mM MES, pH 6.5, 55 mM Propylene Glycol, 56 mM DAB | 860 | 1478 | 866 | 926 | 850 | 238 |
| 59 | 111 mM MES, pH 6.5, 55 mM Propanediol, 56 mM DAB | 1108 | 874 | 812 | 798 | 708 | 216 |
| 60 | 111 mM MES, pH 6.5, 55 mM Diethylene Glycol, 56 mM DAB | 740 | 1098 | 758 | 1150 | 668 | 76 |
| 61 | 111 mM MES, pH 6.5, 55 mM Glycerol, 56 mM DAB | 1136 | 982 | 1428 | 898 | 762 | 96 |
| 62 | 111 mM MES, pH 6.5, 55 mM Ribose, 56 mM DAB | 1466 | 874 | 744 | 1012 | 638 | 382 |
| 63 | 100 mM MES, pH 6.5, 1 mM EDTA, 50 mM DAB | 1018 | 812 | 738 | 756 | 610 | 86 |
| 64 | 100 mM MES, pH 6.5, 1 mM DTPA, 50 mM DAB | 994 | 772 | 738 | 750 | 720 | 86 |
| 65 | 100 mM MES, pH 6.5, 1 mM EGTA, 50 mM DAB | 1028 | 720 | 766 | 740 | 646 | 48 |
| 66 | 100 mM MES, pH 6.5, 1 mM 1,10 Phenanthroline, 50 mM DAB | 784 | 794 | 782 | 784 | 788 | 100 |
| 67 | 100 mM MES, pH 6.5, 1 mM Diethylenetriaminepentamethylene . . . , 50 mM DAB | 1094 | 758 | 738 | 744 | 722 | 162 |
| 68 | 102 mM MES, pH 6.5, 10 mM Acetone, 51 mM DAB | 690 | 736 | 744 | 790 | 738 | 82 |
| 69 | 102 mM MES, pH 6.5, 10 mM Ascorbic Acid, 51 mM DAB | 812 | 802 | 1582 | 850 | 776 | 448 |
| 70 | 100 mM MES, pH 6.5, 1 mM MgCl2, 50 mM DAB | 702 | 798 | 1298 | 700 | 770 | 262 |
| 71 | 100 HEPES, pH 7.4, Nothing, 50 mM DAB | 776 | 1318 | 688 | 660 | 600 | 150 |
| 72 | 111 HEPES, pH 7.4, 55 mM Propylene Glycol, 56 mM DAB | 768 | 1356 | 836 | 780 | 754 | 234 |
| 73 | 111 HEPES, pH 7.4, 55 mM Propanediol, 56 mM DAB | 1210 | 1270 | 784 | 880 | 728 | 184 |
| 74 | 111 HEPES, pH 7.4, 55 mM Diethylene Glycol, 56 mM DAB | 920 | 1640 | 760 | 824 | 708 | 190 |
| 75 | 111 HEPES, pH 7.4, 55 mM Glycerol, 56 mM DAB | 1026 | 788 | 772 | 802 | 730 | 86 |
| 76 | 111 HEPES, pH 7.4, 55 mM Ribose, 56 mM DAB | 880 | 882 | 774 | 806 | 666 | 430 |
| 77 | 100 HEPES, pH 7.4, 1 mM EDTA, 50 mM DAB | 704 | 740 | 658 | 758 | 578 | 156 |
| 78 | 100 HEPES, pH 7.4, 1 mM DTPA, 50 mM DAB | 774 | 712 | 732 | 742 | 684 | 124 |
| 79 | 100 HEPES, pH 7.4, 1 mM EGTA, 50 mM DAB | 720 | 710 | 722 | 746 | 666 | 134 |
| 80 | 100 HEPES, pH 7.4, 1 mM 1,10 Phenanthroline, 50 mM DAB | 912 | 728 | 732 | 714 | 680 | 42 |
| 81 | 100 HEPES, pH 7.4, 1 mM Diethylenetriaminepentamethylene . . . , 50 mM DAB | 776 | 750 | 714 | 726 | 698 | 54 |
| 82 | 102 HEPES, pH 7.4, 10 mM Acetone, 51 mM DAB | 612 | 712 | 720 | 716 | 670 | 138 |
| 83 | 102 HEPES, pH 7.4, 10 mM Ascorbic Acid, 51 mM DAB | 740 | 968 | 1366 | 702 | 750 | 464 |
| 84 | 100 HEPES, pH 7.4, 1 mM MgCl2, 50 mM DAB | 626 | 748 | 1312 | 684 | 670 | 120 |
| 85 | 100 Tris, pH 8.0, Nothing, 50 mM DAB | 622 | 886 | 716 | 692 | 624 | 288 |
| 86 | 111 Tris, pH 8.0, 55 mM Propylene Glycol, 56 mM DAB | 810 | 1242 | 806 | 834 | 790 | 234 |
| 87 | 111 Tris, pH 8.0, 55 mM Propanediol, 56 mM DAB | 826 | 816 | 760 | 808 | 792 | 92 |
| 88 | 111 Tris, pH 8.0, 55 mM Diethylene Glycol, 56 mM DAB | 782 | 1340 | 806 | 834 | 720 | 124 |
| 89 | 111 Tris, pH 8.0, 55 mM Glycerol, 56 mM DAB | 774 | 1368 | 800 | 856 | 816 | 98 |
| 90 | 111 Tris, pH 8.0, 55 mM Ribose, 56 mM DAB | 810 | 856 | 776 | 920 | 648 | 278 |
| 91 | 100 Tris, pH 8.0, 1 mM EDTA, 50 mM DAB | 764 | 848 | 776 | 732 | 646 | 48 |
| 92 | 100 Tris, pH 8.0, 1 mM DTPA, 50 mM DAB | 722 | 718 | 724 | 750 | 742 | 60 |
| 93 | 100 Tris, pH 8.0, 1 mM EGTA, 50 mM DAB | 708 | 782 | 788 | 714 | 648 | 62 |
| 94 | 100 Tris, pH 8.0, 1 mM 1,10 Phenanthroline, 50 mM DAB | 672 | 770 | 748 | 806 | 718 | 50 |
| 95 | 100 Tris, pH 8.0, 1 mM Diethylenetriaminepentamethylene . . . , 50 mM DAB | 676 | 722 | 736 | 742 | 718 | 44 |
| 96 | 102 Tris, pH 8.0, 10 mM Acetone, 51 mM DAB | 708 | 690 | 748 | 698 | 654 | 52 |
| 97 | 102 Tris, pH 8.0, 10 mM Ascorbic Acid, 51 mM DAB | 746 | 820 | 1184 | 754 | 764 | 252 |
| 98 | 100 Tris, pH 8.0, 1 mM MgCl2, 50 mM DAB | 712 | 784 | 1306 | 776 | 640 | 118 |

TABLE 11
Stability of DAB-containing solutions. DAB-containing solutions were incubated at 37° C., and the absorbance at 520 nm was
measured at 0, 2.5, 5 and 18.5 hours of either diluted (0 and 2.5 hours) or undiluted (5 and 18.5 hours). After 18.5 hours of
incubation at 37° C., H₂O₂ was added to a final concentration of 0.03% and the absorbance at 520 nm measured
(“H₂O₂”) following a 200-fold dilution. Each DAB-containing solution was diluted 200-fold in a solution containing 100 mM
imidazole, 0.03% H₂O₂ and 1/20^{th} dilution of HRP polymer (Solution H, Cat. No. 84-0146). The reactions were incubated an
additional 1 hour at 37° C. and the absorbance at 520 nm measured (“Enzyme”).

| | | 0 | 2.5 | 5 | 18.5 | H₂O₂ | Enzyme |
|---|---|---|---|---|---|---|---|
| 1 | Water, Nothing, 50 mM DAB | 0.60 | 2.00 | 0.35 | 1.43 | 3.00 | 0.80 |
| 2 | Water, 55 mM Propylene Glycol, 56 mM DAB | 4.20 | 4.40 | 0.43 | 1.57 | 9.60 | 5.20 |
| 3 | Water, 55 mM Propanediol, 56 mM DAB | 0.80 | 2.00 | 0.42 | 1.80 | 4.20 | 1.40 |
| 4 | Water, 55 mM Diethylene Glycol, 56 mM DAB | 1.00 | 2.00 | 0.43 | 1.80 | 4.80 | 0.80 |
| 5 | Water, 55 mM Glycerol, 56 mM DAB | 0.80 | 1.00 | 0.11 | 0.85 | 3.20 | 1.20 |
| 6 | Water, 55 mM Ribose, 56 mM DAB | 0.80 | 1.60 | 0.34 | 0.94 | 1.40 | 5.60 |
| 7 | Water, 1 mM EDTA, 50 mM DAB | 4.60 | 1.40 | 0.11 | 0.35 | 3.40 | 1.20 |
| 8 | Water, 1 mM DTPA, 50 mM DAB | 1.00 | 1.00 | 0.09 | 0.23 | 2.20 | 1.00 |
| 9 | Water, 1 mM EGTA, 50 mM DAB | 0.60 | 1.60 | 0.34 | 1.41 | 8.60 | 2.40 |
| 10 | Water, 1 mM 1,10 Phenanthroline, 50 mM DAB | 1.00 | 1.40 | 0.16 | 0.58 | 3.00 | 1.00 |
| 11 | Water, 1 mM Diethylenetriaminepentamethylene . . . , 50 mM DAB | 1.00 | 1.80 | 0.37 | 1.51 | 2.60 | 1.40 |
| 12 | Water, 10 mM Acetone, 51 mM DAB | 0.80 | 2.20 | 0.40 | 1.56 | 2.80 | 0.80 |
| 13 | Water, 10 mM Ascorbic Acid, 51 mM DAB | 3.60 | 1.40 | 0.22 | 0.42 | 3.80 | 50.00 |
| 14 | Water, 1 mM MgCl2, 50 mM DAB | 0.60 | 2.40 | 0.39 | 1.60 | 2.40 | 1.40 |
| 15 | 100 mM Citrate, pH 3.0, Nothing, 50 mM DAB | 1.20 | 4.40 | 0.27 | 1.00 | 2.80 | 1.20 |
| 16 | 111 mM Citrate, pH 3.0, 55 mM Propylene Glycol, 56 mM DAB | 3.60 | 5.20 | 0.40 | 1.51 | 4.40 | 3.00 |
| 17 | 111 mM Citrate, pH 3.0, 55 mM Propanediol, 56 mM DAB | 2.00 | 2.00 | 0.30 | 1.17 | 3.20 | 0.60 |
| 18 | 111 mM Citrate, pH 3.0, 55 mM Diethylene Glycol, 56 mM DAB | 1.40 | 2.60 | 0.33 | 1.24 | 4.00 | 1.00 |
| 19 | 111 mM Citrate, pH 3.0, 55 mM Glycerol, 56 mM DAB | 2.00 | 1.40 | 0.09 | 0.43 | 2.60 | 1.00 |
| 20 | 111 mM Citrate, pH 3.0, 55 mM Ribose, 56 mM DAB | 1.40 | 2.00 | 0.20 | 0.75 | 2.40 | 5.80 |
| 21 | 100 mM Citrate, pH 3.0, 1 mM EDTA, 50 mM DAB | 1.20 | 1.60 | 0.20 | 0.79 | 6.40 | 2.00 |
| 22 | 100 mM Citrate, pH 3.0, 1 mM DTPA, 50 mM DAB | 1.00 | 1.80 | 0.06 | 0.11 | 2.80 | 1.00 |
| 23 | 100 mM Citrate, pH 3.0, 1 mM EGTA, 50 mM DAB | 1.00 | 1.60 | 0.14 | 0.52 | 2.20 | 1.00 |
| 24 | 100 mM Citrate, pH 3.0, 1 mM 1,10 Phenanthroline, 50 mM DAB | 1.40 | 2.00 | 0.20 | 0.74 | 3.80 | 1.60 |
| 25 | 100 mM Citrate, pH 3.0, 1 mM Diethylenetriaminepentamethylene . . . , | 1.20 | 1.80 | 0.19 | 0.73 | 2.40 | 1.00 |
| | 50 mM DAB | | | | | | |
| 26 | 102 mM Citrate, pH 3.0, 10 mM Acetone, 51 mM DAB | 1.60 | 2.20 | 0.32 | 1.17 | 2.80 | 1.60 |
| 27 | 102 mM Citrate, pH 3.0, 10 mM Ascorbic Acid, 51 mM DAB | 0.80 | 1.80 | 0.13 | 0.26 | 3.00 | 4.20 |
| 28 | 100 mM Citrate, pH 3.0, 1 mM MgCl2, 50 mM DAB | 5.80 | 2.20 | 0.28 | 1.07 | 3.20 | 1.00 |
| 29 | 100 mM Acetate, pH 4.0, Nothing, 50 mM DAB | 1.60 | 2.60 | 0.32 | 1.22 | 4.20 | 1.20 |
| 30 | 111 mM Acetate, pH 4.0, 55 mM Propylene Glycol, 56 mM DAB | 4.00 | 5.00 | 0.37 | 1.26 | 8.00 | 3.20 |
| 31 | 111 mM Acetate, pH 4.0, 55 mM Propanediol, 56 mM DAB | 2.20 | 2.80 | 0.38 | 1.49 | 4.20 | 1.00 |
| 32 | 111 mM Acetate, pH 4.0, 55 mM Diethylene Glycol, 56 mM DAB | 1.60 | 2.80 | 0.41 | 1.51 | 3.80 | 3.60 |
| 32 | 111 mM Acetate, pH 4.0, 55 mM Glycerol, 56 mM DAB | 2.00 | 1.80 | 0.10 | 0.66 | 3.20 | 1.60 |
| 34 | 111 mM Acetate, pH 4.0, 55 mM Ribose, 56 mM DAB | 0.80 | 2.20 | 0.14 | 0.59 | 2.00 | 2.80 |
| 35 | 100 mM Acetate, pH 4.0, 1 mM EDTA, 50 mM DAB | 1.20 | 2.20 | 0.14 | 0.46 | 6.20 | 2.00 |
| 36 | 100 mM Acetate, pH 4.0, 1 mM DTPA, 50 mM DAB | 1.20 | 1.80 | 0.07 | 0.16 | 4.00 | 1.40 |
| 37 | 100 mM Acetate, pH 4.0, 1 mM EGTA, 50 mM DAB | 1.60 | 2.80 | 0.31 | 1.20 | 4.40 | 1.80 |
| 38 | 100 mM Acetate, pH 4.0, 1 mM 1,10 Phenanthroline, 50 mM DAB | 1.60 | 2.20 | 0.18 | 0.64 | 5.20 | 2.20 |
| 39 | 100 mM Acetate, pH 4.0, 1 mM Diethylenetriaminepentamethylene . . . , | 1.20 | 2.60 | 0.31 | 1.18 | 3.20 | 0.80 |
| | 50 mM DAB | | | | | | |
| 40 | 102 mM Acetate, pH 4.0, 10 mM Acetone, 51 mM DAB | 1.40 | 3.20 | 0.41 | 1.50 | 4.80 | 1.20 |
| 41 | 102 mM Acetate, pH 4.0, 10 mM Ascorbic Acid, 51 mM DAB | 1.80 | 2.40 | 0.19 | 0.37 | 3.60 | 83.00 |
| 42 | 100 mM Acetate, pH 4.0, 1 mM MgCl2, 50 mM DAB | 2.40 | 3.00 | 0.39 | 1.49 | 4.00 | 60.80 |
| 43 | 100 mM Citrate, pH 5.0, Nothing, 50 mM DAB | 2.20 | 4.60 | 0.18 | 0.12 | 1.80 | 63.40 |
| 44 | 111 mM Citrate, pH 5.0, 55 mM Propylene Glycol, 56 mM DAB | 3.60 | 6.20 | 0.34 | 0.53 | 2.20 | 40.00 |
| 45 | 111 mM Citrate, pH 5.0, 55 mM Propanediol, 56 mM DAB | 2.80 | 3.60 | 0.26 | 0.18 | 2.40 | 52.40 |
| 46 | 111 mM Citrate, pH 5.0, 55 mM Diethylene Glycol, 56 mM DAB | 2.60 | 4.20 | 0.30 | 0.19 | 2.00 | 64.00 |
| 47 | 111 mM Citrate, pH 5.0, 55 mM Glycerol, 56 mM DAB | 1.60 | 4.80 | 0.26 | 0.17 | 2.00 | 50.00 |
| 48 | 111 mM Citrate, pH 5.0, 55 mM Ribose, 56 mM DAB | 1.60 | 3.40 | 0.20 | 0.40 | 4.20 | 5.00 |
| 49 | 100 mM Citrate, pH 5.0, 1 mM EDTA, 50 mM DAB | 2.00 | 2.60 | 0.29 | 0.23 | 4.80 | 49.60 |
| 50 | 100 mM Citrate, pH 5.0, 1 mM DTPA, 50 mM DAB | 7.80 | 2.00 | 0.08 | 0.08 | 2.00 | 13.40 |
| 51 | 100 mM Citrate, pH 5.0, 1 mM EGTA, 50 mM DAB | 1.00 | 2.40 | 0.15 | 0.17 | 2.00 | 36.80 |
| 52 | 100 mM Citrate, pH 5.0, 1 mM 1,10 Phenanthroline, 50 mM DAB | 2.20 | 3.60 | 0.30 | 0.27 | 3.60 | 45.00 |
| 53 | 100 mM Citrate, pH 5.0, 1 mM Diethylenetriaminepentamethylene . . . , | 1.80 | 2.20 | 0.07 | 0.06 | 1.60 | 38.20 |
| | 50 mM DAB | | | | | | |
| 54 | 102 mM Citrate, pH 5.0, 10 mM Acetone, 51 mM DAB | 2.80 | 3.00 | 0.27 | 0.17 | 2.00 | 43.20 |
| 55 | 102 mM Citrate, pH 5.0, 10 mM Ascorbic Acid, 51 mM DAB | 1.60 | 2.60 | 0.16 | 0.19 | 1.60 | 40.60 |
| 56 | 100 mM Citrate, pH 5.0, 1 mM MgCl2, 50 mM DAB | 1.60 | 3.20 | 0.21 | 0.13 | 1.40 | 43.00 |
| 57 | 100 mM MES, pH 6.5, Nothing, 50 mM DAB | 2.60 | 3.60 | 0.32 | 0.66 | 2.40 | 4.00 |
| 58 | 111 mM MES, pH 6.5, 55 mM Propylene Glycol, 56 mM DAB | 3.80 | 8.00 | 0.42 | 1.49 | 6.00 | 13.60 |
| 59 | 111 mM MES, pH 6.5, 55 mM Propanediol, 56 mM DAB | 2.20 | 3.40 | 0.34 | 1.25 | 3.20 | 31.20 |
| 60 | 111 mM MES, pH 6.5, 55 mM Diethylene Glycol, 56 mM DAB | 2.20 | 4.40 | 0.36 | 0.55 | 5.20 | 1.20 |
| 61 | 111 mM MES, pH 6.5, 55 mM Glycerol, 56 mM DAB | 2.60 | 3.20 | 0.10 | 0.47 | 2.80 | 1.40 |
| 62 | 111 mM MES, pH 6.5, 55 mM Ribose, 56 mM DAB | 2.20 | 2.80 | 0.17 | 0.49 | 2.80 | 34.60 |
| 63 | 100 mM MES, pH 6.5, 1 mM EDTA, 50 mM DAB | 1.80 | 2.40 | 0.21 | 0.83 | 7.80 | 2.80 |
| 64 | 100 mM MES, pH 6.5, 1 mM DTPA, 50 mM DAB | 2.00 | 5.00 | 0.06 | 0.12 | 3.40 | 1.40 |
| 65 | 100 mM MES, pH 6.5, 1 mM EGTA, 50 mM DAB | 1.40 | 3.40 | 0.16 | 0.56 | 4.00 | 1.60 |
| 66 | 100 mM MES, pH 6.5, 1 mM 1,10 Phenanthroline, 50 mM DAB | 1.80 | 3.20 | 0.22 | 0.79 | 2.60 | 1.60 |
| 67 | 100 mM MES, pH 6.5, 1 mM Diethylenetriaminepentamethylene . . . , 50 mM DAB | 1.80 | 3.00 | 0.19 | 0.67 | 2.80 | 1.60 |
| 68 | 102 mM MES, pH 6.5, 10 mM Acetone, 51 mM DAB | 2.00 | 3.60 | 0.35 | 0.37 | 3.20 | 0.80 |
| 69 | 102 mM MES, pH 6.5, 10 mM Ascorbic Acid, 51 mM DAB | 1.40 | 2.80 | 0.14 | 0.18 | 2.00 | 69.60 |
| 70 | 100 mM MES, pH 6.5, 1 mM MgCl2, 50 mM DAB | 1.60 | 3.60 | 0.33 | 1.18 | 3.20 | 38.00 |
| 71 | 100 HEPES, pH 7.4, Nothing, 50 mM DAB | 1.60 | 6.60 | 0.41 | 0.97 | 2.60 | 25.80 |
| 72 | 111 HEPES, pH 7.4, 55 mM Propylene Glycol, 56 mM DAB | 3.80 | 8.20 | 0.36 | 0.96 | 3.40 | 15.60 |
| 73 | 111 HEPES, pH 7.4, 55 mM Propanediol, 56 mM DAB | 2.60 | 5.20 | 0.47 | 1.14 | 2.00 | 3.20 |
| 74 | 111 HEPES, pH 7.4, 55 mM Diethylene Glycol, 56 mM DAB | 1.60 | 8.20 | 0.48 | 1.19 | 4.60 | 15.40 |
| 75 | 111 HEPES, pH 7.4, 55 mM Glycerol, 56 mM DAB | 2.20 | 3.80 | 0.38 | 0.97 | 3.20 | 1.20 |
| 76 | 111 HEPES, pH 7.4, 55 mM Ribose, 56 mM DAB | 1.80 | 2.80 | 0.13 | 0.30 | 3.20 | 33.20 |
| 77 | 100 HEPES, pH 7.4, 1 mM EDTA, 50 mM DAB | 1.40 | 3.20 | 0.51 | 1.52 | 9.40 | 5.80 |
| 78 | 100 HEPES, pH 7.4, 1 mM DTPA, 50 mM DAB | 1.40 | 3.00 | 0.21 | 0.63 | 2.80 | 1.60 |
| 79 | 100 HEPES, pH 7.4, 1 mM EGTA, 50 mM DAB | 1.40 | 2.80 | 0.14 | 0.38 | 3.40 | 3.20 |
| 80 | 100 HEPES, pH 7.4, 1 mM 1,10 Phenanthroline, 50 mM DAB | 1.40 | 3.80 | 0.53 | 1.71 | 3.60 | 0.80 |
| 81 | 100 HEPES, pH 7.4, 1 mM Diethylenetriaminepentamethylene . . . , 50 mM DAB | 1.40 | 2.80 | 0.22 | 0.24 | 1.80 | 1.20 |
| 82 | 102 HEPES, pH 7.4, 10 mM Acetone, 51 mM DAB | 1.40 | 3.80 | 0.47 | 1.11 | 2.40 | 1.40 |
| 83 | 102 HEPES, pH 7.4, 10 mM Ascorbic Acid, 51 mM DAB | 1.20 | 3.00 | 0.19 | 0.26 | 2.20 | 73.20 |
| 84 | 100 HEPES, pH 7.4, 1 mM MgCl2, 50 mM DAB | 1.40 | 4.00 | 0.45 | 1.09 | 2.80 | 13.60 |
| 85 | 100 Tris, pH 8.0, Nothing, 50 mM DAB | 1.40 | 3.80 | 0.30 | 0.58 | 3.00 | 88.40 |
| 86 | 111 Tris, pH 8.0, 55 mM Propylene Glycol, 56 mM DAB | 4.00 | 5.60 | 0.30 | 1.12 | 4.20 | 11.60 |
| 87 | 111 Tris, pH 8.0, 55 mM Propanediol, 56 mM DAB | 1.40 | 3.40 | 0.35 | 1.22 | 3.60 | 1.40 |
| 88 | 111 Tris, pH 8.0, 55 mM Diethylene Glycol, 56 mM DAB | 1.60 | 5.00 | 0.36 | 1.25 | 2.60 | 1.20 |
| 89 | 111 Tris, pH 8.0, 55 mM Glycerol, 56 mM DAB | 1.80 | 3.00 | 0.09 | 0.42 | 3.60 | 1.40 |
| 90 | 111 Tris, pH 8.0, 55 mM Ribose, 56 mM DAB | 0.80 | 2.80 | 0.17 | 0.58 | 2.80 | 18.20 |
| 91 | 100 Tris, pH 8.0, 1 mM EDTA, 50 mM DAB | 1.00 | 2.80 | 0.22 | 0.79 | 14.20 | 2.60 |
| 92 | 100 Tris, pH 8.0, 1 mM DTPA, 50 mM DAB | 1.40 | 2.60 | 0.06 | 0.11 | 4.00 | 1.20 |
| 93 | 100 Tris, pH 8.0, 1 mM EGTA, 50 mM DAB | 5.80 | 2.60 | 0.17 | 0.58 | 4.40 | 2.80 |
| 94 | 100 Tris, pH 8.0, 1 mM 1,10 Phenanthroline, 50 mM DAB | 1.40 | 3.00 | 0.22 | 0.73 | 4.40 | 0.60 |
| 95 | 100 Tris, pH 8.0, 1 mM Diethylenetriaminepentamethylene . . . , 50 mM DAB | 1.40 | 2.40 | 0.20 | 0.67 | 3.40 | 1.00 |
| 96 | 102 Tris, pH 8.0, 10 mM Acetone, 51 mM DAB | 1.40 | 3.40 | 0.34 | 1.14 | 3.20 | 2.60 |
| 97 | 102 Tris, pH 8.0, 10 mM Ascorbic Acid, 51 mM DAB | 0.60 | 6.00 | 0.13 | 0.25 | 2.80 | 34.40 |
| 98 | 100 Tris, pH 8.0, 1 mM MgCl2, 50 mM DAB | 1.20 | 3.20 | 0.31 | 1.07 | 2.60 | 4.80 |

TABLE 12
Stability of DAB Formulations. DAB was formulated in a variety of buffers and incubated at 37° C. At the indicated time,
absorbance spectra were obtained using a NanoDrop ND-1000 spectrometer. The absorbance values at 520 nm and the
ratio of the 520 nm absorbance at each time to that prior to incubation (“0 Hrs”) are shown.

| | | Absorbance 520 nm | | | | | | | | | | |
|---|---|---|---|---|---|---|---|---|---|---|---|---|
| | | 0 | 11.8 | | 36.1 | | 62.7 | | 86.2 | | 110.8 | |
| | | Hrs | Hrs | Ratio | Hrs | Ratio | Hrs | Ratio | Hrs | Ratio | Hrs | Ratio |
| 1 | 200 mM Sodium Citrate, pH 3.0, 50 mM DAB | 0.028 | 0.321 | 11.5 | 1.267 | 45.3 | 2.16 | 77.1 | 2.55 | 91.1 | 2.65 | 94.6 |
| 2 | 200 mM Sodium Acetate, pH 4.0, 50 mM DAB | 0.036 | 0.514 | 14.3 | 2.37 | 65.8 | 4.44 | 123.3 | 5.67 | 157.5 | 6.79 | 188.6 |
| 4 | 200 mM MES, pH 6.5, 50 mM DAB | 0.033 | 0.575 | 17.4 | 1.052 | 31.9 | 1.37 | 41.5 | 1.58 | 47.9 | 1.77 | 53.6 |
| 5 | 10 mM Polyethylene Glycol, 50 mM DAB | 0.043 | 0.375 | 8.7 | 1.747 | 40.6 | 3.64 | 84.7 | 4.98 | 115.8 | 6.23 | 144.9 |
| 6 | 10 mM Propanediol, 50 mM DAB | 0.039 | 0.59 | 15.1 | 2.486 | 63.7 | 5.34 | 136.9 | 7.06 | 181.0 | 8.56 | 219.5 |
| 7 | 10 mM Glycerol, 50 mM DAB | 0.036 | 0.487 | 13.5 | 2.487 | 69.1 | 4.94 | 137.2 | 6.58 | 182.8 | 8 | 222.2 |
| 8 | 10 mM Diethylene Glycol, 50 mM DAB | 0.038 | 0.362 | 9.5 | 1.247 | 32.8 | 5.27 | 138.7 | 6.87 | 180.8 | 8.26 | 217.4 |
| 9 | 10 mM D-Ribose, 50 mM DAB | 0.035 | 0.09 | 2.6 | 0.154 | 4.4 | 0.19 | 5.4 | 0.22 | 6.3 | 0.29 | 8.3 |
| 10 | 10 mM Ascorbic Acid, 50 mM DAB | 0.034 | 0.209 | 6.1 | 0.442 | 13.0 | 0.84 | 24.7 | 1.36 | 40.0 | 2.27 | 66.8 |
| 11 | 10 mM Acetonitrile, 50 mM DAB | 0.042 | 0.635 | 15.1 | 2.69 | 64.0 | 5.41 | 128.8 | 7.09 | 168.8 | 8.42 | 200.5 |
| 12 | 1 mM Sodium Metabisulfite, 50 mM DAB | 0.039 | 0.197 | 5.1 | 1.234 | 31.6 | 3.58 | 91.8 | 5.57 | 142.8 | 7.29 | 186.9 |
| 13 | 1 mM EGTA, pH 7.6, 50 mM DAB | 0.035 | 0.507 | 14.5 | 2.368 | 67.7 | 4.69 | 134.0 | 6.43 | 183.7 | 7.87 | 224.9 |
| 14 | 1 mM DTPA, pH 4, 50 mM DAB | 0.038 | 0.081 | 2.1 | 0.346 | 9.1 | 0.78 | 20.5 | 1.21 | 31.8 | 1.68 | 44.2 |
| 15 | 1 mM EDTA, pH 8, 50 mM DAB | 0.038 | 0.119 | 3.1 | 0.565 | 14.9 | 1.26 | 33.2 | 1.95 | 51.3 | 2.63 | 69.2 |
| 16 | 1 mM 1,10-Phenanthroline, 50 mM DAB | 0.038 | 0.184 | 4.8 | 0.868 | 22.8 | 1.91 | 50.3 | 2.83 | 74.5 | 3.69 | 97.1 |
| 17 | 1 mM Diethylenetriaminepentamethylenephosphonic | 0.032 | 0.488 | 15.3 | 2.238 | 69.9 | 4.47 | 139.7 | 6.05 | 189.1 | 7.21 | 225.3 |
| | acid, 50 mM DAB | | | | | | | | | | | |
| 18 | 10 mM Ethanolamine, 50 mM DAB | 0.038 | 0.57 | 15.0 | 2.395 | 63.0 | 5.01 | 131.8 | 6.75 | 177.6 | 8.27 | 217.6 |
| 19 | 10 mM Imidazole, 50 mM DAB | 0.036 | 0.598 | 16.6 | 2.523 | 70.1 | 5.21 | 144.7 | 6.96 | 193.3 | 8.36 | 232.2 |
| 20 | 10 mM 1M Hydroxyquinone in Ethanol, | 0.04 | 0.527 | 13.2 | 2.231 | 55.8 | 4.41 | 110.3 | 6 | 150.0 | 7.3 | 182.5 |
| | 50 mM DAB | | | | | | | | | | | |
| 21 | 75% Methanol, 50 mM DAB | 0.054 | 0.087 | 1.6 | 0.154 | 2.9 | 0.2 | 3.7 | 0.37 | 6.9 | 0.41 | 7.6 |
| 22 | 50% Methanol, 50 mM DAB | 0.045 | 0.177 | 3.9 | 0.601 | 13.4 | 1 | 22.2 | 1.62 | 36.0 | 1.94 | 43.1 |
| 23 | 25% Methanol, 50 mM DAB | 0.043 | 0.479 | 11.1 | 1.792 | 41.7 | 3.12 | 72.6 | 4.32 | 100.5 | 4.94 | 114.9 |
| 24 | 10% Methanol, 50 mM DAB | 0.037 | 0.488 | 13.2 | 1.956 | 52.9 | 3.57 | 96.5 | 4.73 | 127.8 | 5.56 | 150.3 |
| 25 | Water, 50 mM DAB | 0.038 | 0.589 | 15.5 | 1.516 | 39.9 | 5.23 | 137.6 | 7.25 | 190.8 | 8.74 | 230.0 |
| 26 | 200 mM DAB 85% Methanol | 0.152 | 0.144 | 0.9 | 0.187 | 1.2 | 0.21 | 1.4 | 0.32 | 2.1 | 0.31 | 2.0 |
| 27 | Control Kit | 0.677 | 1.287 | 1.9 | 2.895 | 4.3 | 3.46 | 5.1 | 2.45 | 3.6 | 2.51 | 3.7 |

TABLE 13
Stability of DAB Formulations.

| | Slope |
|---|---|
| 200 mM DAB 85% Methanol | 0.0017 |
| 10 mM D-Ribose, 50 mM DAB | 0.0021 |
| 75% Methanol, 50 mM DAB | 0.0033 |
| 200 mM MES, pH 6.5, 50 mM DAB | 0.0146 |
| Control Kit | 0.0146 |
| 1 mM DTPA, pH 4, 50 mM DAB | 0.0151 |
| 50% Methanol, 50 mM DAB | 0.0177 |
| 10 mM Ascorbic Acid, 50 mM DAB | 0.0189 |
| 1 mM EDTA, pH 8, 50 mM DAB | 0.024 |
| 200 mM Sodium Citrate, pH 3.0, 50 mM DAB | 0.0256 |
| 1 mM 1,10-Phenanthroline, 50 mM DAB | 0.0341 |
| 25% Methanol, 50 mM DAB | 0.0464 |
| 10% Methanol, 50 mM DAB | 0.0522 |
| 10 mM Polyethylene Glycol, 50 mM DAB | 0.0583 |
| 200 mM Sodium Acetate, pH 4.0, 50 mM DAB | 0.0639 |
| 1 mM Diethylenetriaminepentamethylenephosphonic | 0.0684 |
| acid, 50 mM DAB | |
| 10 mM 1M Hydroxyquinone in Ethanol, 50 mM DAB | 0.0685 |
| 1 mM Sodium Metabisulfite, 50 mM DAB | 0.0688 |
| 1 mM EGTA, pH 7.6, 50 mM DAB | 0.074 |
| 10 mM Glycerol, 50 mM DAB | 0.0756 |
| 10 mM Ethanolamine, 50 mM DAB | 0.0778 |
| 10 mM Imidazole, 50 mM DAB | 0.0791 |
| 10 mM Acetonitrile, 50 mM DAB | 0.0798 |
| 10 mM Propanediol, 50 mM DAB | 0.081 |
| 10 mM Diethylene Glycol, 50 mM DAB | 0.0813 |
| Water, 50 mM DAB | 0.0843 |

DAB was formulated in a variety of buffers and incubated at 37° C. The absorbance at 520 nm was plotted as a function of time, as demonstrated in FIG. 2 and the slope of a regression line drawn through the data points determined. The data in the table are ordered according to the slopes.

TABLE 14
Stability of DAB Formulations. DAB was formulated in a variety of buffers and incubated at 37° C. At the
indicated time, absorbance spectra were obtained using a NanoDrop ND-1000 spectrometer. The
absorbance values at 520 nm and the ratio of the 520 nm absorbance at each time to that prior to incubation
(“0 Hrs”) are shown.

| | Absorbance 520 nm | | | | | Ratio | | | |
|---|---|---|---|---|---|---|---|---|---|
| | 0 hrs | 12.2 hrs | 41.4 hrs | 62.3 hrs | 84.8 hrs | 12.2 hrs | 41.4 hrs | 62.3 hrs | 84.8 hrs |
| 0.5 mM DTPA, pH 4, 50 mM DAB | 0.885 | 1.481 | 3 | 3.93 | 4.77 | 1.7 | 3.4 | 4.4 | 5.39 |
| 1.0 mM DTPA, pH 4, 50 mM DAB | 0.898 | 1.366 | 2.57 | 3.32 | 4.03 | 1.5 | 2.9 | 3.7 | 4.49 |
| 2.0 mM DTPA, pH 4, 50 mM DAB | 0.884 | 1.257 | 2.13 | 2.67 | 3.24 | 1.4 | 2.4 | 3.0 | 3.67 |
| 5.0 mM DTPA, pH 4, 50 mM DAB | 0.851 | 1.166 | 1.79 | 2.17 | 2.59 | 1.4 | 2.1 | 2.5 | 3.04 |
| 10.0 mM DTPA, pH 4, 50 mM DAB | 0.835 | 1.183 | 1.87 | 2.31 | 2.78 | 1.4 | 2.2 | 2.8 | 3.33 |
| 0.5 mM EDTA, pH 8, 50 mM DAB | 0.865 | 1.503 | 3.17 | 4.13 | 5.05 | 1.7 | 3.7 | 4.8 | 5.84 |
| 1.0 mM EDTA, pH 8, 50 mM DAB | 0.878 | 1.533 | 3.19 | 4.15 | 5.04 | 1.7 | 3.6 | 4.7 | 5.74 |
| 2.0 mM EDTA, pH 8, 50 mM DAB | 0.874 | 1.665 | 3.66 | 4.84 | 5.82 | 1.9 | 4.2 | 5.5 | 6.66 |
| 5.0 mM EDTA, pH 8, 50 mM DAB | 0.863 | 1.839 | 4.33 | 5.54 | 6.69 | 2.1 | 5.0 | 6.4 | 7.75 |
| 10.0 mM EDTA, pH 8, 50 mM DAB | 0.866 | 2.111 | 4.76 | 6.00 | 7.16 | 2.4 | 5.5 | 6.9 | 8.27 |
| 0.5 mM 1,10-Phenanthroline, 50 mM DAB | 0.886 | 1.796 | 4.06 | 5.22 | 6.26 | 2.0 | 4.6 | 5.9 | 7.07 |
| 1.0 mM 1,10-Phenanthroline, 50 mM DAB | 0.904 | 1.808 | 3.99 | 5.12 | 6.1 | 2.0 | 4.4 | 5.7 | 6.75 |
| 2.0 mM 1,10-Phenanthroline, 50 mM DAB | 0.899 | 1.79 | 3.9 | 5.04 | 6.01 | 2.0 | 4.3 | 5.6 | 6.69 |
| 5.0 mM 1,10-Phenanthroline, 50 mM DAB | 0.612 | 1.695 | 3.59 | 4.72 | 5.52 | 2.8 | 5.9 | 7.7 | 9.02 |
| 10.0 mM 1,10-Phenanthroline, 50 mM DAB | 0.946 | 1.8 | 3.82 | 5.07 | 5.92 | 1.9 | 4.0 | 5.4 | 6.26 |
| 5.0 mM Ribose, 50 mM DAB | 0.873 | 0.772 | 1.18 | 1.84 | 2.51 | 0.9 | 1.4 | 2.1 | 2.88 |
| 10.0 mM Ribose, 50 mM DAB | 0.862 | 0.643 | 1.04 | 1.13 | 1.34 | 0.7 | 1.2 | 1.3 | 1.55 |
| 20.0 mM Ribose, 50 mM DAB | 0.842 | 0.508 | 0.96 | 1.09 | 1.2 | 0.6 | 1.1 | 1.3 | 1.43 |
| 50.0 mM Ribose, 50 mM DAB | 0.794 | 0.56 | 1.16 | 1.38 | 1.38 | 0.7 | 1.5 | 1.7 | 1.74 |
| 100.0 mM Ribose, 50 mM DAB | 0.718 | 0.669 | 1.49 | 1.88 | 2.11 | 0.9 | 2.1 | 2.6 | 2.94 |
| 5.0 mM Polyethylene Glycol, 50 mM DAB | 0.906 | 2.364 | 5.43 | 6.86 | 8.09 | 2.6 | 6.0 | 7.6 | 8.93 |
| 10.0 mM Polyethylene Glycol, 50 mM DAB | 0.87 | 2.086 | 4.74 | 6.22 | 7.37 | 2.4 | 5.4 | 7.1 | 8.47 |
| 20.0 mM Polyethylene Glycol, 50 mM DAB | 0.755 | 1.553 | 3.78 | 4.90 | 6.04 | 2.1 | 5.0 | 6.5 | 8.00 |
| 50.0 mM Polyethylene Glycol, 50 mM DAB | 0.751 | 1.151 | 1.8 | 2.52 | 2.81 | 1.5 | 2.4 | 3.4 | 3.74 |
| 5.0 mM Diethylene Glycol, 50 mM DAB | 0.876 | 2.733 | 6.12 | 7.89 | 9.08 | 3.1 | 7.0 | 9.0 | 10.37 |
| 10.0 mM Diethylene Glycol, 50 mM DAB | 0.892 | 2.641 | 6.13 | 7.87 | 9.05 | 3.0 | 6.9 | 8.8 | 10.15 |
| 20.0 mM Diethylene Glycol, 50 mM DAB | 0.893 | 2.521 | 6.02 | 7.64 | 8.89 | 2.8 | 6.7 | 8.6 | 9.96 |
| 50.0 mM Diethylene Glycol, 50 mM DAB | 0.887 | 2.487 | 5.86 | 7.50 | 8.6 | 2.8 | 6.6 | 8.5 | 9.70 |
| 100.0 mM Diethylene Glycol, 50 mM DAB | 0.88 | 2.472 | 5.49 | 7.04 | 8.2 | 2.8 | 6.2 | 8.0 | 9.32 |
| 0.5 mM Sodium Metabisulfite, 50 mM DAB | 0.888 | 1.811 | 5.2 | 7.06 | 8.46 | 2.0 | 5.9 | 8.0 | 9.53 |
| 1.0 mM Sodium Metabisulfite, 50 mM DAB | 0.889 | 1.678 | 4.8 | 6.69 | 8.18 | 1.9 | 5.4 | 7.5 | 9.20 |
| 2.0 mM Sodium Metabisulfite, 50 mM DAB | 0.876 | 1.454 | 3.04 | 4.60 | 6.23 | 1.7 | 3.5 | 5.3 | 7.11 |
| 5.0 mM Sodium Metabisulfite, 50 mM DAB | 0.862 | 1.213 | 1.97 | 2.31 | 2.59 | 1.4 | 2.3 | 2.7 | 3.00 |
| 10.0 mM Sodium Metabisulfite, 50 mM DAB | 0.838 | 1.127 | 1.57 | 1.71 | 1.8 | 1.3 | 1.9 | 2.0 | 2.15 |
| 5.0 mM Ascorbic Acid, 50 mM DAB | 0.894 | 1.167 | 1.52 | 1.84 | 2.23 | 1.3 | 1.7 | 2.1 | 2.49 |
| 10.0 mM Ascorbic Acid, 50 mM DAB | 0.891 | 1.149 | 1.51 | 1.85 | 2.36 | 1.3 | 1.7 | 2.1 | 2.65 |
| 20.0 mM Ascorbic Acid, 50 mM DAB | 0.884 | 1.136 | 1.53 | 1.91 | 2.31 | 1.3 | 1.7 | 2.2 | 2.61 |
| 50.0 mM Ascorbic Acid, 50 mM DAB | 0.884 | 1.085 | 1.5 | 1.89 | 2.31 | 1.2 | 1.7 | 2.1 | 2.61 |
| 100.0 mM Ascorbic Acid, 50 mM DAB | 0.757 | 1.004 | 1.04 | 1.35 | 1.5 | 1.3 | 1.4 | 1.8 | 1.98 |

TABLE 15
Stability of DAB Formulations. DAB was formulated in a variety of buffers and incubated at 37° C. At the indicated time (listed in
hours), absorbance spectra were obtained using a NanoDrop ND-1000 spectrometer. The absorbance values at 520 nm and the
ratio of the 520 nm absorbance at each time to that prior to incubation (“0 Hrs”) are shown.

| | | Absorbance (520 nm) | | | | | | | Ratio Abs₅₂₀ₙₘ Time 0/Time T | | | | | |
|---|---|---|---|---|---|---|---|---|---|---|---|---|---|---|
| | | Time (Hrs) | | | | | | | | | | | | |
| | | 0 | 13.35 | 36.53 | 59.28 | 90.1 | 158 | 395 | 13.35 | 36.53 | 59.28 | 90.1 | 158 | 395 |
| 1 | 1 mM DTPA, 1 mM Ribose, 50 mM DAB | 0.029 | 0.037 | 0.097 | 0.254 | 0.54 | 1.38 | 4.8 | 1.3 | 3.3 | 8.8 | 18.6 | 47.6 | 165.5 |
| 2 | 1 mM DTPA, 1 mM Ascorbic Acid, | 0.033 | 0.069 | 0.093 | 0.234 | 0.33 | 0.486 | 1.24 | 2.1 | 2.8 | 7.1 | 10.0 | 14.7 | 37.6 |
| | 50 mM DAB | | | | | | | | | | | | | |
| 3 | 1 mM DTPA, 1 mM Sodium Metabisulfite, | 0.033 | 0.055 | 0.088 | 0.247 | 0.44 | 1.21 | 5.73 | 1.7 | 2.7 | 7.5 | 13.3 | 36.7 | 173.6 |
| | 50 mM DAB | | | | | | | | | | | | | |
| 4 | 1 mM DTPA, 10 mM Ribose, 50 mM DAB | 0.034 | 0.113 | 0.084 | 0.191 | 0.22 | 0.413 | 2.61 | 3.3 | 2.5 | 5.6 | 6.5 | 12.1 | 76.8 |
| 5 | 1 mM DTPA, 10 mM Ascorbic Acid, | 0.034 | 0.094 | 0.091 | 0.478 | 0.93 | 2.64 | 5.59 | 2.8 | 2.7 | 14.1 | 27.4 | 77.6 | 164.4 |
| | 50 mM DAB | | | | | | | | | | | | | |
| 6 | 1 mM DTPA, 10 mM Sodium Metabisulfite, | 0.031 | 0.043 | 0.053 | 0.084 | 0.1 | 0.173 | 0.74 | 1.4 | 1.7 | 2.7 | 3.2 | 5.6 | 23.9 |
| | 50 mM DAB | | | | | | | | | | | | | |
| 7 | 10 mM DTPA, 1 mM Ribose, 50 mM DAB | 0.031 | 0.041 | 0.046 | 0.069 | 0.11 | 0.272 | 1.53 | 1.3 | 1.5 | 2.2 | 3.5 | 8.8 | 49.4 |
| 8 | 10 mM DTPA, 1 mM Ascorbic Acid, | 0.031 | 0.047 | 0.046 | 0.097 | 0.14 | 0.263 | 0.8 | 1.5 | 1.5 | 3.1 | 4.5 | 8.5 | 25.8 |
| | 50 mM DAB | | | | | | | | | | | | | |
| 9 | 10 mM DTPA, 1 mM Sodium Metabisulfite, | 0.034 | 0.039 | 0.055 | 0.079 | 0.12 | 0.218 | 1.09 | 1.1 | 1.6 | 2.3 | 3.5 | 6.4 | 32.1 |
| | 50 mM DAB | | | | | | | | | | | | | |
| 10 | 10 mM DTPA, 10 mM Ribose, 50 mM DAB | 0.032 | 0.121 | 0.085 | 0.191 | 0.22 | 0.511 | 3.32 | 3.8 | 2.7 | 6.0 | 6.9 | 16.0 | 103.8 |
| 11 | 10 mM DTPA, 10 mM Ascorbic Acid, | 0.031 | 0.061 | 0.063 | 0.317 | 0.71 | 2.31 | 3.61 | 2.0 | 2.0 | 10.2 | 22.9 | 74.5 | 116.5 |
| | 50 mM DAB | | | | | | | | | | | | | |
| 12 | 10 mM DTPA, 10 mM Sodium Metabisulfite, | 0.029 | 0.036 | 0.045 | 0.061 | 0.08 | 0.121 | 0.44 | 1.2 | 1.6 | 2.1 | 2.8 | 4.2 | 15.2 |
| | 50 mM DAB | | | | | | | | | | | | | |
| 13 | 1 mM Ribose, 1 mM DTPA, 50 mM DAB | 0.035 | 0.04 | 0.09 | 0.369 | 0.75 | 1.73 | 5.75 | 1.1 | 2.6 | 10.5 | 21.4 | 49.4 | 164.3 |
| 14 | 1 mM Ribose, 1 mM Ascorbic Acid, | 0.031 | 0.089 | 0.123 | 0.494 | 0.62 | 0.822 | 1.62 | 2.9 | 4.0 | 15.9 | 20.0 | 26.5 | 52.3 |
| | 50 mM DAB | | | | | | | | | | | | | |
| 15 | 1 mM Ribose, 1 mM Sodium Metabisulfite, | 0.033 | 0.042 | 0.615 | 2.72 | 4.59 | 7.74 | 13.7 | 1.3 | 18.6 | 82.4 | 139.1 | 234.5 | 415.2 |
| | 50 mM DAB | | | | | | | | | | | | | |
| 16 | 1 mM Ribose, 10 mM DTPA, 50 mM DAB | 0.043 | 0.047 | 0.052 | 0.083 | 0.11 | 0.252 | 1.43 | 1.1 | 1.2 | 1.9 | 2.6 | 5.9 | 33.3 |
| 17 | 1 mM Ribose, 10 mM Ascorbic Acid, | 0.032 | 0.09 | 0.131 | 0.517 | 0.97 | 2.8 | 1.37 | 2.8 | 4.1 | 16.2 | 30.3 | 87.5 | 42.8 |
| | 50 mM DAB | | | | | | | | | | | | | |
| 18 | 1 mM Ribose, 10 mM Sodium Metabisulfite, | 0.039 | 0.041 | 0.047 | 0.071 | 0.1 | 0.15 | 0.53 | 1.1 | 1.2 | 1.8 | 2.6 | 3.8 | 13.6 |
| | 50 mM DAB | | | | | | | | | | | | | |
| 19 | 10 mM Ribose, 1 mM DTPA, 50 mM DAB | 0.032 | 0.121 | 0.08 | 0.185 | 0.21 | 0.425 | 2.75 | 3.8 | 2.5 | 5.8 | 6.6 | 13.3 | 85.9 |
| 20 | 10 mM Ribose, 1 mM Ascorbic Acid, | 0.034 | 0.12 | 0.059 | 0.204 | 0.27 | 0.475 | 1.35 | 3.5 | 1.7 | 6.0 | 7.9 | 14.0 | 39.7 |
| | 50 mM DAB | | | | | | | | | | | | | |
| 21 | 10 mM Ribose, 1 mM Sodium Metabisulfite, | 0.033 | 0.13 | 0.177 | 0.19 | 0.21 | 0.418 | 2.68 | 3.9 | 5.4 | 5.8 | 6.4 | 12.7 | 81.2 |
| | 50 mM DAB | | | | | | | | | | | | | |
| 22 | 10 mM Ribose, 10 mM DTPA, 50 mM DAB | 0.032 | 0.116 | 0.124 | 0.181 | 0.21 | 0.489 | 3.01 | 3.6 | 3.9 | 5.7 | 6.6 | 15.3 | 94.1 |
| 23 | 10 mM Ribose, 10 mM Ascorbic Acid, | 0.036 | 0.128 | 0.144 | 0.297 | 0.58 | 2.11 | 2.35 | 3.6 | 4.0 | 8.3 | 16.1 | 58.6 | 65.3 |
| | 50 mM DAB | | | | | | | | | | | | | |
| 24 | 10 mM Ribose, 10 mM Sodium Metabisulfite, | 0.04 | 0.041 | 0.047 | 0.051 | 0.05 | 0.105 | 0.42 | 1.0 | 1.2 | 1.3 | 1.3 | 2.6 | 10.5 |
| | 50 mM DAB | | | | | | | | | | | | | |
| 25 | 1 mM Ascorbic Acid, 1 mM DTPA, | 0.035 | 0.073 | 0.15 | 0.24 | 0.34 | 0.522 | 1.18 | 2.1 | 4.3 | 6.9 | 9.7 | 14.9 | 33.7 |
| | 50 mM DAB | | | | | | | | | | | | | |
| 26 | 1 mM Ascorbic Acid, 1 mM Ribose, | 0.039 | 0.101 | 0.398 | 0.592 | 0.71 | 0.906 | 1.92 | 2.6 | 10.2 | 15.2 | 18.2 | 23.2 | 49.2 |
| | 50 mM DAB | | | | | | | | | | | | | |
| 27 | 1 mM Ascorbic Acid, 1 mM Sodium | 0.036 | 0.123 | 0.222 | 0.341 | 0.46 | 0.629 | 1.48 | 3.4 | 6.2 | 9.5 | 12.8 | 17.5 | 41.1 |
| | Metabisulfite, 50 mM DAB | | | | | | | | | | | | | |
| 28 | 1 mM Ascorbic Acid, 10 mM DTPA, | 0.042 | 0.046 | 0.068 | 0.098 | 0.15 | 0.261 | 0.79 | 1.1 | 1.6 | 2.3 | 3.6 | 6.2 | 18.8 |
| | 50 mM DAB | | | | | | | | | | | | | |
| 29 | 1 mM Ascorbic Acid, 10 mM Ribose, | 0.036 | 0.11 | 0.171 | 0.202 | 0.27 | 0.467 | 1.35 | 3.1 | 4.8 | 5.6 | 7.5 | 13.0 | 37.5 |
| | 50 mM DAB | | | | | | | | | | | | | |
| 30 | 1 mM Ascorbic Acid, 10 mM Sodium | 0.034 | 0.091 | 0.183 | 0.256 | 0.37 | 0.374 | 0.3 | 2.7 | 5.4 | 7.5 | 10.9 | 11.0 | 8.8 |
| | Metabisulfite, 50 mM DAB | | | | | | | | | | | | | |
| 31 | 10 mM Ascorbic Acid, 1 mM DTPA, | 0.031 | 0.099 | 0.243 | 0.483 | 0.93 | 2.64 | 4.67 | 3.2 | 7.8 | 15.6 | 30.0 | 85.2 | 150.6 |
| | 50 mM DAB | | | | | | | | | | | | | |
| 32 | 10 mM Ascorbic Acid, 1 mM Ribose, | 0.038 | 0.094 | 0.267 | 0.527 | 1.01 | 2.73 | 3.01 | 2.5 | 7.0 | 13.9 | 26.6 | 71.8 | 79.2 |
| | 50 mM DAB | | | | | | | | | | | | | |
| 33 | 10 mM Ascorbic Acid, 1 mM Sodium | 0.035 | 0.122 | 0.15 | 0.528 | 0.93 | 2.53 | 2.87 | 3.5 | 4.3 | 15.1 | 26.6 | 72.3 | 82.0 |
| | Metabisulfite, 50 mM DAB | | | | | | | | | | | | | |
| 34 | 10 mM Ascorbic Acid, 10 mM DTPA, | 0.034 | 0.059 | 0.142 | 0.323 | 0.73 | 2.27 | 4.35 | 1.7 | 4.2 | 9.5 | 21.5 | 66.8 | 127.9 |
| | 50 mM DAB | | | | | | | | | | | | | |
| 35 | 10 mM Ascorbic Acid, 10 mM Ribose, | 0.033 | 0.129 | 0.197 | 0.272 | 0.54 | 1.71 | 3.3 | 3.9 | 6.0 | 8.2 | 16.4 | 51.8 | 100.0 |
| | 50 mM DAB | | | | | | | | | | | | | |
| 36 | 10 mM Ascorbic Acid, 10 mM Sodium | 0.035 | 0.133 | 0.317 | 0.549 | 0.67 | 1.04 | 4.11 | 3.8 | 9.1 | 15.7 | 19.1 | 29.7 | 117.4 |
| | Metabisulfite, 50 mM DAB | | | | | | | | | | | | | |
| 37 | 1 mM Sodium Metabisulfite, 1 mM DTPA, | 0.035 | 0.06 | 0.141 | 0.254 | 0.44 | 1.18 | 6 | 1.7 | 4.0 | 7.3 | 12.6 | 33.7 | 171.4 |
| | 50 mM DAB | | | | | | | | | | | | | |
| 38 | 1 mM Sodium Metabisulfite, 1 mM Ribose, | 0.034 | 0.055 | 1.02 | 2.48 | 4.27 | 7.19 | 12.8 | 1.6 | 30.0 | 72.9 | 125.6 | 211.5 | 376.5 |
| | 50 mM DAB | | | | | | | | | | | | | |
| 39 | 1 mM Sodium Metabisulfite, 1 mM Ascorbic | 0.036 | 0.118 | 0.236 | 0.303 | 0.41 | 0.606 | 1.24 | 3.3 | 6.6 | 8.4 | 11.4 | 16.8 | 34.4 |
| | Acid, 50 mM DAB | | | | | | | | | | | | | |
| 40 | 1 mM Sodium Metabisulfite, 10 mM DTPA, | 0.043 | 0.043 | 0.06 | 0.084 | 0.11 | 0.237 | 1.02 | 1.0 | 1.4 | 2.0 | 2.6 | 5.5 | 23.7 |
| | 50 mM DAB | | | | | | | | | | | | | |
| 41 | 1 mM Sodium Metabisulfite, 10 mM Ribose, | 0.032 | 0.124 | 0.179 | 0.191 | 0.2 | 0.428 | 2.59 | 3.9 | 5.6 | 6.0 | 6.3 | 13.4 | 80.9 |
| | 50 mM DAB | | | | | | | | | | | | | |
| 42 | 1 mM Sodium Metabisulfite, 10 mM Ascorbic | 0.034 | 0.118 | 0.176 | 0.519 | 0.95 | 2.63 | 4.01 | 3.5 | 5.2 | 15.3 | 27.9 | 77.4 | 117.9 |
| | Acid, 50 mM DAB | | | | | | | | | | | | | |
| 43 | 10 mM Sodium Metabisulfite, 1 mM DTPA, | 0.03 | 0.046 | 0.066 | 0.081 | 0.08 | 0.162 | 0.68 | 1.5 | 2.2 | 2.7 | 2.7 | 5.4 | 22.7 |
| | 50 mM DAB | | | | | | | | | | | | | |
| 44 | 10 mM Sodium Metabisulfite, 1 mM Ribose, | 0.036 | 0.039 | 0.043 | 0.056 | 0.06 | 0.12 | 0.44 | 1.1 | 1.2 | 1.6 | 1.7 | 3.3 | 12.2 |
| | 50 mM DAB | | | | | | | | | | | | | |
| 45 | 10 mM Sodium Metabisulfite, 1 mM Ascorbic | 0.034 | 0.096 | 0.179 | 0.233 | 0.32 | 0.367 | 0.31 | 2.8 | 5.3 | 6.9 | 9.4 | 10.8 | 9.1 |
| | Acid, 50 mM DAB | | | | | | | | | | | | | |
| 46 | 10 mM Sodium Metabisulfite, 10 mM DTPA, | 0.033 | 0.04 | 0.051 | 0.064 | 0.06 | 0.121 | 0.44 | 1.2 | 1.5 | 1.9 | 1.8 | 3.7 | 13.3 |
| | 50 mM DAB | | | | | | | | | | | | | |
| 47 | 10 mM Sodium Metabisulfite, 10 mM Ribose, | 0.028 | 0.042 | 0.048 | 0.051 | 0.04 | 0.09 | 0.41 | 1.5 | 1.7 | 1.8 | 1.4 | 3.2 | 14.6 |
| | 50 mM DAB | | | | | | | | | | | | | |
| 48 | 10 mM Sodium Metabisulfite, 10 mM Ascorbic | 0.032 | 0.125 | 0.279 | 0.52 | 0.65 | 0.885 | 3.79 | 3.9 | 8.7 | 16.3 | 20.3 | 27.7 | 118.4 |
| | Acid, 50 mM DAB | | | | | | | | | | | | | |
| 49 | 1 mM DTPA, 50 mM DAB | 0.036 | 0.092 | 0.331 | 0.654 | 1.12 | 2.34 | 6.85 | 2.6 | 9.2 | 18.2 | 31.1 | 65.0 | 190.3 |
| 50 | 10 mM DTPA, 50 mM DAB | 0.044 | 0.048 | 0.084 | 0.133 | 0.2 | 0.463 | 1.86 | 1.1 | 1.9 | 3.0 | 4.5 | 10.5 | 42.3 |
| 51 | 1 mM Ribose, 50 mM DAB | 0.035 | 0.131 | 1.446 | 2.99 | 4.89 | 8.05 | 13.85 | 3.7 | 41.3 | 85.4 | 139.7 | 230.0 | 395.7 |
| 52 | 10 mM Ribose, 50 mM DAB | 0.034 | 0.127 | 0.188 | 0.196 | 0.21 | 0.446 | 2.75 | 3.7 | 5.5 | 5.8 | 6.2 | 13.1 | 80.9 |
| 53 | 1 mM Ascorbic Acid, 50 mM DAB | 0.037 | 0.227 | 0.626 | 0.822 | 0.95 | 1.2 | 2.42 | 6.1 | 16.9 | 22.2 | 25.7 | 32.4 | 65.4 |
| 54 | 10 mM Ascorbic Acid, 50 mM DAB | 0.036 | 0.184 | 0.369 | 0.659 | 1.28 | 3.25 | 6.56 | 5.1 | 10.3 | 18.3 | 35.6 | 90.3 | 182.2 |
| 55 | 1 mM Sodium Metabisulfite, 50 mM DAB | 0.038 | 0.232 | 1.062 | 2.56 | 5.09 | 9.25 | 15.05 | 6.1 | 27.9 | 67.4 | 133.9 | 243.4 | 396.1 |
| 56 | 10 mM Sodium Metabilufite, 50 mM DAB | 0.034 | 0.072 | 0.135 | 0.191 | 0.23 | 0.376 | 1.06 | 2.1 | 4.0 | 5.6 | 6.8 | 11.1 | 31.2 |
| 57 | Nothing, 50 mM DAB | 0.041 | 0.677 | 2.366 | 4.11 | 6.48 | 11.05 | 16.3 | 16.5 | 57.7 | 100.2 | 158.0 | 269.5 | 397.6 |
| 58 | 50 mM DAB, 5 mM HCl, 85% Methanol, | 0.122 | 0.157 | 0.181 | 0.209 | 0.27 | 0.438 | 0.87 | 1.3 | 1.5 | 1.7 | 2.2 | 3.6 | 7.1 |
| | 50 mM DAB | | | | | | | | | | | | | |

TABLE 16
Stability of DAB Formulations.

| | Time (hours) | 90.1 Hrs |
|---|---|---|
| 1 | 1 mM DTPA, 1 mM Ribose, 50 mM DAB | Deep Maroon, no precipitation |
| 2 | 1 mM DTPA, 1 mM Ascorbic Acid, 50 mM DAB | Orange-Brown, no precipitation |
| 3 | 1 mM DTPA, 1 mM Sodium Metabisulfite, 50 mM DAB | Maroon, no precipitation |
| 4 | 1 mM DTPA, 10 mM Ribose, 50 mM DAB | Brown, no precipitation |
| 5 | 1 mM DTPA, 10 mM Ascorbic Acid, 50 mM DAB | Very Dark Brown, No precipitation |
| 6 | 1 mM DTPA, 10 mM Sodium Metabisulfite, 50 mM DAB | Light Pink, small brown precipitate on wall |
| 7 | 10 mM DTPA, 1 mM Ribose, 50 mM DAB | Orange-Brown, no precipitation |
| 8 | 10 mM DTPA, 1 mM Ascorbic Acid, 50 mM DAB | Darker Orange-Brown, no precipitation |
| 9 | 10 mM DTPA, 1 mM Sodium Metabisulfite, 50 mM DAB | Light Pink, no precipitation |
| 10 | 10 mM DTPA, 10 mM Ribose, 50 mM DAB | Brown, no precipitation |
| 11 | 10 mM DTPA, 10 mM Ascorbic Acid, 50 mM DAB | Very Dark Brown, No precipitation |
| 12 | 10 mM DTPA, 10 mM Sodium Metabisulfite, 50 mM DAB | Light Pink, precipitation on walls |
| 13 | 1 mM Ribose, 1 mM DTPA, 50 mM DAB | Very dark maroon, no precipitation |
| 14 | 1 mM Ribose, 1 mM Ascorbic Acid, 50 mM DAB | Dark Orange-Brown, no precipitation |
| 15 | 1 mM Ribose, 1 mM Sodium Metabisulfite, 50 mM DAB | Very Very Dark purple, no precipitation |
| 16 | 1 mM Ribose, 10 mM DTPA, 50 mM DAB | Light Orange-Brown, no precipitation |
| 17 | 1 mM Ribose, 10 mM Ascorbic Acid, 50 mM DAB | Very Dark Brown, No precipitation |
| 18 | 1 mM Ribose, 10 mM Sodium Metabisulfite, 50 mM DAB | Light Pink, some brown precipitation |
| 19 | 10 mM Ribose, 1 mM DTPA, 50 mM DAB | Orange-Brown, no precipitation |
| 20 | 10 mM Ribose, 1 mM Ascorbic Acid, 50 mM DAB | Orange-Brown, no precipitation |
| 21 | 10 mM Ribose, 1 mM Sodium Metabisulfite, 50 mM DAB | Orange-Brown, no precipitation |
| 22 | 10 mM Ribose, 10 mM DTPA, 50 mM DAB | Orange-Brown, no precipitation |
| 23 | 10 mM Ribose, 10 mM Ascorbic Acid, 50 mM DAB | Orange-Brown, no precipitation |
| 24 | 10 mM Ribose, 10 mM Sodium Metabisulfite, 50 mM DAB | Lighter yellow-brown, white precipitation |
| 25 | 1 mM Ascorbic Acid, 1 mM DTPA, 50 mM DAB | Darker Orange-Brown, no precipitation |
| 26 | 1 mM Ascorbic Acid, 1 mM Ribose, 50 mM DAB | Dark Orange-Brown, no precipitation |
| 27 | 1 mM Ascorbic Acid, 1 mM Sodium Metabisulfite, 50 mM DAB | Dark Orange-Brown, no precipitation |
| 28 | 1 mM Ascorbic Acid, 10 mM DTPA, 50 mM DAB | Lighter yellow-brown, no precipitation |
| 29 | 1 mM Ascorbic Acid, 10 mM Ribose, 50 mM DAB | Light brown, no precipitation |
| 30 | 1 mM Ascorbic Acid, 10 mM Sodium Metabisulfite, 50 mM DAB | Orange-Brown, no precipitation |
| 31 | 10 mM Ascorbic Acid, 1 mM DTPA, 50 mM DAB | Dark Brown, no precipitation |
| 32 | 10 mM Ascorbic Acid, 1 mM Ribose, 50 mM DAB | Dark Brown, no precipitation |
| 33 | 10 mM Ascorbic Acid, 1 mM Sodium Metabisulfite, 50 mM DAB | Dark Brown, no precipitation |
| 34 | 10 mM Ascorbic Acid, 10 mM DTPA, 50 mM DAB | Dark Brown, no precipitation |
| 35 | 10 mM Ascorbic Acid, 10 mM Ribose, 50 mM DAB | Dark Brown, no precipitation |
| 36 | 10 mM Ascorbic Acid, 10 mM Sodium Metabisulfite, 50 mM DAB | Dark Brown, brown precipitation |
| 37 | 1 mM Sodium Metabisulfite, 1 mM DTPA, 50 mM DAB | Purple, no precipitation |
| 38 | 1 mM Sodium Metabisulfite, 1 mM Ribose, 50 mM DAB | Very Very Dark Purple, no precipitation |
| 39 | 1 mM Sodium Metabisulfite, 1 mM Ascrobic Acid, 50 mM DAB | Orange-Brown, no precipitation |
| 40 | 1 mM Sodium Metabisulfite, 10 mM DTPA, 50 mM DAB | Light Pink, no precipitation |
| 41 | 1 mM Sodium Metabisulfite, 10 mM Ribose, 50 mM DAB | Orange-Brown, no precipitation |
| 42 | 1 mM Sodium Metabisulfite, 10 mM Ascrobic Acid, 50 mM DAB | Very Dark Brown, No precipitation |
| 43 | 10 mM Sodium Metabisulfite, 1 mM DTPA, 50 mM DAB | Light pink, small purple precipitate |
| 44 | 10 mM Sodium Metabisulfite, 1 mM Ribose, 50 mM DAB | Light Pink, precipitate |
| 45 | 10 mM Sodium Metabisulfite, 1 mM Ascrobic Acid, 50 mM DAB | Orange-Brown, no precipitation |
| 46 | 10 mM Sodium Metabisulfite, 10 mM DTPA, 50 mM DAB | Light Pink, small precipitate |
| 47 | 10 mM Sodium Metabisulfite, 10 mM Ribose, 50 mM DAB | Light yellow, precipitate |
| 48 | 10 mM Sodium Metabisulfite, 10 mM Ascrobic Acid, 50 mM DAB | Dark brown, brown precipitate |
| 49 | 1 mM DTPA, 50 mM DAB | Dark purple, no precipitation |
| 50 | 10 mM DTPA, 50 mM DAB | Light purple, no precipitate |
| 51 | 1 mM Ribose, 50 mM DAB | Very dark purple, no precipitate |
| 52 | 10 mM Ribose, 50 mM DAB | Light yellow, no precipitate |
| 53 | 1 mM Ascorbic Acid, 50 mM DAB | Purple-brown, no precipitate |
| 54 | 10 mM Ascorbic Acid, 50 mM DAB | Dark brown, no precipitate |
| 55 | 1 mM Sodium Metabisulfite, 50 mM DAB | Very dark purple, no precipitate |
| 56 | 10 mM Sodium Metabilufite, 50 mM DAB | Pink, light precipitate |
| 57 | Nothing, 50 mM DAB | Very dark purple, no precipitate |

Physical description of DAB-containing solutions listed in Table 15 after 90.1 hrs of incubation at 37° C. DAB was formulated in a variety of buffers and incubated at 37° C.

TABLE 17
Effect of antioxidant on DAB stability.

| 50 mM DAB, | | | | | | |
|---|---|---|---|---|---|---|
| 10 mM DTPA, | | | | | | |
| 65 Propylene | Absorbance 520 nm | | | | | |
| Glycol with: | 0 | 13 h | 94 h | 132 h | 309 h | |
| 1 mM Sodium | 0.036 | 0.058 | 0.114 | 0.152 | 0.152 | |
| Metabisulfite | | | | | | |
| 10 mM Sodium | 0.039 | 0.052 | 0.106 | 0.139 | 0.139 | Precipi- |
| Metabisulfite | | | | | | tate |
| 20 mM Sodium | 0.033 | 0.04 | 0.089 | 0.103 | 0.103 | Precipi- |
| Metabisulfite | | | | | | tate |
| 50 mM Sodium | 0.012 | 0.087 | 0.073 | 0.088 | 0.088 | Precipi- |
| Metabisulfite | | | | | | tate |
| 1 mM Sodium | 0.043 | 0.054 | 0.106 | 0.136 | 0.136 | |
| Sulfite | | | | | | |
| 10 mM Sodium | 0.045 | 0.06 | 0.115 | 0.152 | 0.152 | |
| Sulfite | | | | | | |
| 20 mM Sodium | 0.046 | 0.058 | 0.118 | 0.151 | 0.151 | |
| Sulfite | | | | | | |
| 50 mM Sodium | 0.042 | 0.053 | 0.096 | 0.115 | 0.115 | |
| Sulfite | | | | | | |

TABLE 18
Effect of DAB Concentration on Signal Intensity*

| | | Final DAB | Signal |
|---|---|---|---|
| | | conc. | Intensity |
| Slide | DAB Source | (mM) | Score |
| 1 | Invitrogen-Zymed (85-9143) | 2.5 | 2.5 |
| 2 | Next Gen. Formulation | 2.5 | 2.75 |
| 3 | Next Gen. Formulation | 3.75 | 3.25 |
| 4 | Next Gen. Formulation | 5 | 3.75 |
| 5 | Next Gen. Formulation | 6.25 | 4.0 |
| 6 | Vision ImmPact ™ (SK-4105) | NA^{#} | 4.0 |
| 7 | Biocare Cardassian (DBC859L10) | NA | 3.25 |
| 8 | PowerVision+ ™ | NA | 2.5 |
| | (DPVB + 110DAB) | | |
| 9 | Pierce (34002) | NA | 0.5 |

*IHC was performed on colon cancer tissue specimens (137734) and stained for expression of CEA using anti-CEA antibody (Invitrogen-Zymed #08-1057) and the Invitrogen-Zymed Broad Spectrum Secondary Antibody (#85-9043). The Next Generation DAB (250 mM DAB, 10 mM DTPA, 1 mM Sodium Sulfite, 65% Propylene glycol) and Hydrogen Peroxide Buffer (200 mM Sodium Acetate pH 5, 50 mM Imidazole, 1 mM DTPA, 0.03% Hydrogen Peroxide) were mixed <15 minutes before use.
^{#}Not Available

TABLE 19
Effect of buffer and DAB on HRP activity.

| | | Max at |
|---|---|---|
| | HRP Activity | 5 min |
| | ΔAbs₄₆₅/sec | Abs₄₆₅ |
| Invitrogen Kit Reagents | 0.00390 | 0.3330 |
| 200 mM NaOAc, pH 5.0, 1 mM DTPA, | 0.00470 | 0.2690 |
| 50 mM Imidazole 0.03% H₂O₂, | | |
| Kit DAB | | |
| 200 mM NaOAc, pH 5.0, 1 mM DTPA, | 0.00520 | 0.2570 |
| 50 mM Imidazole 0.03% H₂O₂, | | |
| 50 mM DAB | | |
| 100 mM Imidazole, 0.015% H₂O₂, | 0.00765 | 0.1980 |
| 50 mM DAB | | |
| 100 mM Imidazole, Kit H₂O₂, | 0.00780 | 0.2085 |
| 50 mM DAB | | |
| 200 mM Imidazole, 0.015% H₂O₂, | 0.00830 | 0.3025 |
| 50 mM DAB | | |
| 100 mM Imidazole, 0.005% H₂O₂, | 0.00323 | 0.1655 |
| 50 mM DAB | | |
| 100 mM Imidazole, 0.001% H₂O₂, | 0.00095 | 0.0675 |
| 50 mM DAB | | |
| 100 mM Imidazole, 0.030% H₂O₂, | 0.00695 | 0.1965 |
| 50 mM DAB | | |

HRP (9.3 μg/mL) was assayed in 200 mM Sodium Acetate, pH 5, 1.5 mM DAB, 0.015% H₂O₂, 0.2% Gelatin with the indicated buffer and sources of DAB.

TABLE 20
Effect of buffer on hydrogen peroxide stability.

| | Time 0 | | 112 hours | | |
|---|---|---|---|---|---|
| | HRP | Signal | HRP | Signal | Activity |
| | Activity | 5 min | Activity | 5 min | 112 h/Tₒ |
| 200 mM Sodium Citrate, pH 3.0, 0.03% H₂O₂ | 0.0120 | 1.32 | 0.0162 | 1.65 | 136% |
| 200 mM Sodium Citrate, pH 4.0, 0.03% H₂O₂ | 0.0178 | 0.88 | 0.0142 | 1.20 | 80% |
| 200 mM Sodium Acetate, pH 4.0, 0.03% H₂O₂ | 0.0097 | 0.83 | 0.0137 | 1.30 | 141% |
| 200 mM Sodium Citrate, pH 5.0, 0.03% H₂O₂ | 0.0065 | 0.71 | 0.0081 | 1.01 | 126% |
| 200 mM Sodium Acetate, pH 5.0, 0.03% H₂O₂ | 0.0063 | 0.81 | 0.0090 | 1.30 | 144% |
| 200 mM Sodium Citrate, pH 6.0, 0.03% H₂O₂ | 0.0054 | 0.88 | 0.0072 | 1.22 | 132% |
| 200 mM Sodium Phosphate, pH 6.0, 0.03% H₂O₂ | 0.0054 | 0.85 | 0.0064 | 1.16 | 119% |
| 200 mM MES, pH 6.5, 0.03% H₂O₂ | 0.0041 | 0.81 | 0.0011 | 0.11 | 26% |
| 200 mM Sodium Phosphate, pH 7.0, 0.03% H₂O₂ | 0.0027 | 0.51 | 0.0031 | 0.65 | 115% |
| 200 mM HEPES, pH 7.4, 0.03% H₂O₂ | 0.0016 | 0.39 | 0.0003 | 0.03 | 16% |
| 200 mM Tris, ph 8.0, 0.03% H₂O₂ | 0.0008 | 0.16 | 0.0025 | 0.53 | 306% |
| 200 mM Sodium Bicarbonate, 0.03% H₂O₂ | 0.0003 | 0.04 | 0.0004 | 0.05 | 140% |
| 50 mM Ammonium Chloride, 0.03% H₂O₂ | 0.0016 | 0.21 | 0.0012 | 0.11 | 75% |
| 50 mM Imidazole, 0.03% H₂O₂ | 0.0236 | 0.61 | 0.0115 | 0.90 | 49% |
| 50 mM 6-Aminocaproic Acid, 0.03% H₂O₂ | | | 0.0065 | 0.86 | |
| 50 mM Ethanolamine, 0.03% H₂O₂ | | | 0.0012 | 0.11 | |
| 10 mM Polyethylene Glycol, 0.03% H₂O₂ | | | 0.0013 | 0.30 | |
| 10 mM Propanediol, 0.03% H₂O₂ | | | 0.0021 | 0.48 | |
| 10 mM Diethylene Glycol, 0.03% H₂O₂ | | | 0.0018 | 0.37 | |
| 10 mM Glycerol, 0.03% H₂O₂ | | | 0.0020 | 0.55 | |
| 10 mM D-Ribose, 0.03% H₂O₂ | 0.0026 | 0.48 | 0.0016 | 0.30 | 61% |
| 10 mM Sodium Metabisulfite, 0.03% H₂O₂ | | | 0.0005 | 0.30 | |
| 10 mM Acetonitrile, 0.03% H₂O₂ | | | 0.0020 | 0.51 | |
| 10 mM Ascorbic Acid, 0.03% H₂O₂ | | | 0.0006 | 0.04 | |
| Kit Buffer | 0.0023 | 0.53 | 0.0021 | 0.56 | 91% |
| Water | 0.0028 | 0.57 | 0.0020 | 0.50 | 71% |

TABLE 21
Effect of DAB concentration and presence of imidazole on HRP activity.

| | | Max at |
|---|---|---|
| | HRP Activity | 5 min |
| | ΔAbs₄₆₅/sec | Abs₄₆₅ |
| 200 mM NaAc, pH 5.0, 1 mM DTPA, 50 mM Imidazole, | 0.0039 | 0.18 |
| 0.5 mM DAB, 0.015% H₂O₂ | | |
| 200 mM NaAc, pH 5.0, 1 mM DTPA, 50 mM Imidazole, | 0.0051 | 0.30 |
| 1.0 mM DAB, 0.015% H₂O₂ | | |
| 200 mM NaAc, pH 5.0, 1 mM DTPA, 50 mM Imidazole, | 0.0036 | 0.27 |
| 1.25 mM DAB, 0.015% H₂O₂ | | |
| 200 mM NaAc, pH 5.0, 1 mM DTPA, 50 mM Imidazole, | 0.0038 | 0.47 |
| 0.05% Gelatin 1.5 mM DAB, 0.015% H₂O₂ | | |
| 100 mM NaAc, pH 5.0, 1.5 mM DAB, 0.015% H₂O₂, | 0.0036 | 0.56 |
| 0.02% Gelatin | | |
| 200 mM NaAc, pH 5.0, 1 mM DTPA, 1.5 mM DAB, | 0.0026 | 0.40 |
| 0.015% H₂O₂, 0.02% Gelatin | | |
| 100 mM NaAc, pH 5.0, 1.5 mM DAB, 0.015% H₂O₂ | 0.0033 | 0.43 |
| 100 mM NaAc, pH 5.0, 1.5 mM DAB, 0.015% H₂O₂, 0.2% Gelatin | 0.0038 | 0.58 |
| 200 mM NaAc, pH 5.0, 1.5 mM DAB, 0.015% H₂O₂, 0.2% Gelatin | 0.0036 | 0.65 |
| 200 mM NaAc, pH 5.0, 0.5 mM DAB, 0.015% H₂O₂, 0.2% Gelatin | 0.0042 | 0.26 |
| 200 mM NaAc, pH 5.0, 1.0 mM DAB, 0.015% H₂O₂, 0.2% Gelatin | 0.0052 | 0.49 |
| 200 mM NaAc, pH 5.0, 2.0 mM DAB, 0.015% H₂O₂, 0.2% Gelatin | 0.0031 | 0.60 |
| 200 mM NaAc, pH 5.0, 3.0 mM DAB, 0.015% H₂O₂, 0.2% Gelatin | 0.0026 | 0.47 |
| 200 mM NaAc, pH 5.0, 6.0 mM DAB, 0.015% H₂O₂, 0.2% Gelatin | 0.0024 | 0.40 |

The data are the values shown in FIG. 8.

TABLE 22
Effect of imidazole concentration on HRP activity.

| | ΔAbs_{465 nm}/sec | Abs_{465 nm} |
|---|---|---|
| 200 mM NaAc, pH 5.0, 1.5 mM DAB, | 0.003 | 0.66 |
| 0.015% H2O2, 0.2% Gelatin | | |
| 200 mM NaAc, pH 5.0, 1.5 mM DAB, | 0.004 | 0.71 |
| 20 mM Imidazole, 0.015% H2O2, | | |
| 0.2% Gelatin | | |
| 200 mM NaAc, pH 5.0, 1.5 mM DAB, | 0.007 | 0.74 |
| 50 mM Imidazole, 0.015% H2O2, | | |
| 0.2% Gelatin | | |
| 200 mM NaAc, pH 5.0, 1.5 mM DAB, | 0.007 | 0.70 |
| 100 mM Imidazole, 0.015% H2O2, | | |
| 0.2% Gelatin | | |
| 200 mM NaAc, pH 5.0, 1.5 mM DAB, | 0.009 | 0.58 |
| 200 mM Imidazole, 0.015% H2O2, | | |
| 0.2% Gelatin | | |
| 200 mM NaAc, pH 5.0, 1.5 mM DAB, | 0.011 | 0.57 |
| 400 mM Imidazole, 0.015% H2O2, | | |
| 0.2% Gelatin | | |

TABLE 23
Effect of additives on HRP activity.

| | ΔAbs_{465 nm}/sec | Abs_{465 nm} |
|---|---|---|
| 200 mM NaAc, pH 5.0, 1.5 mM DAB, | 0.003 | 0.66 |
| 0.015% H2O2, 0.2% Gelatin | | |
| 200 mM NaAc, pH 5.0, 1.5 mM DAB, | 0.005 | 0.68 |
| 10 mM Fumarate, 0.015% H2O2, | | |
| 0.2% Gelatin | | |
| 200 mM NaAc, pH 5.0, 1.5 mM DAB, | 0.009 | 0.50 |
| 10% Dextran Sulfate, 0.015% H2O2, | | |
| 0.2% Gelatin | | |
| 200 mM NaAc, pH 5.0, 1.5 mM DAB, | 0.009 | 0.56 |
| 5% Dextran Sulfate, 0.015% H2O2, | | |
| 0.2% Gelatin | | |
| 200 mM NaAc, pH 5.0, 1.5 mM DAB, | 0.008 | 0.50 |
| 2.5% Dextran Sulfate, 0.015% H2O2, | | |
| 0.2% Gelatin | | |
| 200 mM NaAc, pH 5.0, 1.5 mM DAB, | 0.004 | 0.57 |
| 1% Dextran Sulfate, 0.015% H2O2, | | |
| 0.2% Gelatin | | |
| 200 mM NaAc, pH 5.0, 1.5 mM DAB, | 0.010 | 0.82 |
| 5% Dextran Sulfate, 100 mM | | |
| Imidazole 0.015% H2O2, | | |
| 0.2% Gelatin | | |
| 200 mM NaAc, pH 5.0, 1.5 mM DAB, | 0.008 | 0.72 |
| 5% Dextran Sulfate, 100 mM | | |
| Imidazole 10 mM Fumarate, | | |
| 0.015% H2O2, 0.2% Gelatin | | |
| 200 mM NaAc, pH 5.0, 1.5 mM DAB, | 0.006 | 0.65 |
| 5% Dextran Sulfate, 10 mM Fumarate, | | |
| 0.015% H2O2 | | |
| 200 mM NaAc, pH 5.0, 1.5 mM DAB, | 0.011 | 0.43 |
| 1% Dextran Sulfate, 10 mM Fumarate, | | |
| 0.015% H2O2, 2 mM Nickel Chloride | | |

TABLE 24
Description of Chromogen and pH of DAB detection kits from different suppliers.

| Source | Buffer | Volume | Chrom Description | pH | Volume | Combination |
|---|---|---|---|---|---|---|
| ZytoDot SPEC HER2 Probe Kit (Cat No. C-3003) | 7.4 | 1000 uL | Intense Black | | 50 uL | 7.2 |
| Spot-Light CISH HER-2 (84-0146) | 6.1 | 50 uL | Brown | | 50 uL | 4.3 |
| Pierce DAB Substrate Kit (Prod #34002) | 5.5 | 1000 uL | Clear | | 100 uL | 5.5 |
| Vector ImmPact ™ DAB (Cat. No. SK-4105) | 6.6 | 1000 uL | Very Light Pink | 1.1 | 100 uL | 4.2 |
| BioCare Medical Betazoid DAB Chromogen Kit (Cat. No. BDB2004H) | 6.4 | 1000 uL | Black | | 100 uL | 4.0 |
| Diagnostics Biosystems DAB Plus (Cat. No. K0478) | 7.5 | 1000 uL | Very Light Brown | | 100 uL | 6.2 |
| Powervision ™ ImmunoVision Technologies Inc (Cat. No. DPVB + 110 DAB) | 4.5 | 100 uL | Light Yellow | 0.4 | 50 uL | 4.4 |
| Powervision ™ Immunovision Chromogene ™ Polymer-HRP ISH Detection Kit | 4.6 | 100 uL | Light Yellow | | 100 uL | 4.4 |

TABLE 25
Effect of buffer and DAB on HRP activity.

| | HRP | Max at |
|---|---|---|
| | Activity | 5 min |
| | ΔAbs₄₆₅/sec | Abs₄₆₅ |
| Invitrogen Reagents | 0.00055 | 0.1605 |
| PowerVision+ ™ Histostaining Kit | 0.00870 | 0.6830 |
| PowerVision+ ™ Histostaining Kit | 0.00945 | 0.6640 |
| Vector VIP | 0.00645 | 0.8030 |
| IMMPact ™ DAB | 0.01005 | 1.1740 |
| Invitrogen Kit Reagents | 0.00390 | 0.3330 |
| 200 mM NaOAc, pH 5.0, 1 mM DTPA, | 0.00520 | 0.2570 |
| 50 mM Imidazole 0.03% H₂O₂, | | |
| 1.5 mM DAB | | |
| 200 mM NaOAc, pH 5.0, 1 mM DTPA, | 0.00810 | 0.3445 |
| 50 mM Imidazole 0.03% H₂O₂, | | |
| PowerVision+ ™ DAB | | |
| PowerVision+ ™ Buffer 1.5 mM DAB | 0.00650 | 0.8780 |
| 200 mM NaOAc, pH 5.0, 1 mM DTPA, | 0.00495 | 0.2510 |
| 50 mM Imidazole 0.03% H₂O₂, | | |
| Impact DAB | | |
| IMMPact ™ diluent, 1.5 mM DAB | 0.01130 | 0.9870 |

HRP (9.3 μg/mL) was assayed in 200 mM Sodium Acetate, pH 5, 1.5 mM DAB, 0.015% H₂O₂, 0.2% Gelatin with the indicated buffer and sources of DAB.

TABLE 26
Lot-to-Lot Reproducibility of Next Generation DAB Chromogen

| | | | | Signal |
|---|---|---|---|---|
| Slide | Tissue | 1° Ab^{#} | DAB* | Intensity |
| 1 | Breast | Estrogen Receptor | Prototype | 3.75 |
| | Carcinoma | (#08-0149) | Lot 1 | |
| 2 | Breast | Estrogen Receptor | Prototype | 3.75 |
| | Carcinoma | (#08-0149) | Lot 2 | |
| 3 | Breast | Estrogen Receptor | Prototype | 4.0 |
| | Carcinoma | (#08-0149) | Lot 3 | |
| 4 | Breast | Estrogen Receptor | SuperPicture | 3.25 |
| | Carcinoma | (#08-0149) | #87-9663 | |
| 5 | Breast | Ki67 (#08-1129) | Prototype | 3.25 |
| | Carcinoma | | Lot 1 | |
| 6 | Breast | Ki67 (#08-1129) | Prototype | 3.5 |
| | Carcinoma | | Lot 2 | |
| 7 | Breast | Ki67 (#08-1129) | Prototype | 3.25 |
| | Carcinoma | | Lot 3 | |
| 8 | Breast | Ki67 (#08-1129) | SuperPicture | 3.0 |
| | Carcinoma | | #87-9663 | |

*All prototype lots of DAB were formulated as follows: 200 mM DAB, 20 mM HCl, 10 mM DTPA, 1 mM Sodium Sulfite, 65% Propylene Glycol. The DAB formulation was diluted 1/40 in the prototype Hydrogen Peroxide Buffer (200 mM Sodium Acetate pH 5.5, 50 mM Imidazole, 1 mM DTPA, 0.03% Hydrogen Peroxide).
^{#}IHC was performed to detect expression of Estrogen Receptor and Ki67 according to the manufacturer's instructions.

TABLE 27
Intra-run Reproducibility of Next Generation Detection Reagents*

| | | Signal Intensity Score^{#} | | | |
|---|---|---|---|---|---|
| Sample | Tissue | Slide 1 | Slide 2 | Slide 3 | Mean ± SD |
| 1 | 10. Colon Adenocarcinoma | 0.0 | 0.5 | 0.5 | 0.33 ± 0.29 |
| 2 | 22. Colon Adenocarcinoma | 4.0 | 3.5 | 3.75 | 3.75 ± 0.25 |
| 3 | 29. Colon Adenocarcinoma | 3.25 | 3.0 | 3.25 | 3.17 ± 0.14 |
| 4 | 31. Colon Adenocarcinoma | 3.5 | 3.5 | 3.5 | 3.50 ± 0.00 |
| 5 | 35. Colon Adenocarcinoma | 3.75 | 3.25 | 3.5 | 3.50 ± 0.25 |
| 6 | 41. Colon Adenocarcinoma | 2.5 | 2.5 | 2.5 | 2.50 ± 0.00 |
| 7 | 45. Colon Adenocarcinoma | 4.0 | 3.25 | 3.25 | 3.50 ± 0.43 |
| 8 | 47. Colon Adenocarcinoma | 3.5 | 3.0 | 3.5 | 3.33 ± 0.29 |
| 9 | 48. Colon Adenocarcinoma | 3.5 | 3.5 | 3.5 | 3.50 ± 0.00 |
| 10 | 53. Colon Mucinous Carcinoma | 3.25 | 3.25 | 3.25 | 3.25 ± 0.00 |
| 11 | 57. Signet Ring Adenocarcinoma | 3.5 | 3.5 | 3.75 | 3.58 ± 0.14 |
| 12 | 59. Signet Ring Adenocarcinoma | 3.25 | 3.5 | 3.75 | 3.50 ± 0.25 |

*IHC was performed using anti-CEA (Invitrogen C/N 080057) primary antibody developed with a prototype Next Generation SuperPicture ™ Detection Kit tested in triplicate across 12 tissue samples.
^{#}Staining intensity was scored following Invitrogen Quality Procedures, Document No. TM-041. Maximum staining intensity score = 4.0

TABLE 28
Day-to-Day Reproducibility of Next Generation Detection Platform

| | | Signal Intensity Score | | | |
|---|---|---|---|---|---|
| Sample | Human Tissue | Day 1 | Day 2 | Day 3 | Mean ± SD |
| 1 | Cervix | 3.0 | 3.5 | 3.0 | 3.17 ± 0.29 |
| 2 | Esophagus | 3.25 | 1.5 | 3.25 | 2.67 ± 1.01 |
| 3 | Kidney | 3.25 | 3.25 | 3.25 | 3.25 ± 0.00 |
| 4 | Liver | 3.75 | 3.5 | 3.5 | 3.58 ± 0.14 |
| 5 | Pancreas | 3.25 | 3.5 | 3.25 | 3.33 ± 0.14 |
| 6 | Small Intestine | 3.75 | 3.25 | 3.75 | 3.58 ± 0.29 |
| 7 | Tonsil | 3.75 | 3.5 | 3.5 | 3.58 ± 0.14 |
| 8 | Tonsil -Ch6 | 3.75 | 3.0 | 3.75 | 3.50 ± 0.43 |
| 9 | Uterus | 3.75 | 4.0 | 3.5 | 3.75 ± 0.25 |
| 10 | Skin | 2.75 | 2.75 | 2.75 | 2.75 ± 0.00 |

*IHC was performed using anti-PCNA (Invitrogen C/N 081110) primary antibody and developed with a prototype Next Generation SuperPicture ™ Detection Kit repeated on 3 different days.
^{#}Staining intensity was scored following Invitrogen Quality Procedures, Document No. TM-041. Score range = 0.0-4.0

TABLE 29
DAB Raw Material Comparison*

| | | | DAB | DAB For- | Signal |
|---|---|---|---|---|---|
| Slide | Tissue | 1° Ab^{#} | Source* | mulation | Intensity# |
| 1 | Tonsil | PCNA | Aldrich | Next Gen. | 3.25 |
| | | (#08-1110) | D12384 | | |
| 2 | Tonsil | PCNA | Aldrich | Next Gen. | 3.25 |
| | | (#08-1110) | D12384 | | |
| 3 | Tonsil | PCNA | Sigma | Next Gen. | 3.5 |
| | | (#08-1110) | D5637 | | |
| 4 | Tonsil | PCNA | Sigma | Next Gen. | 3.5 |
| | | (#08-1110) | D5637 | | |
| 5 | Tonsil | PCNA | Aldrich | Next Gen. | 3.75 |
| | | (#08-1110) | 261890 | | |
| 6 | Tonsil | PCNA | Aldrich | Next Gen. | 3.75 |
| | | (#08-1110) | 261890 | | |
| 7 | Tonsil | PCNA | Fluka | Next Gen. | 3.75 |
| | | (#08-1110) | 32750 | | |
| 8 | Tonsil | PCNA | Fluka | Next Gen. | 3.25 |
| | | (#08-1110) | 32750 | | |
| 11 | Tonsil | PCNA | NA | Zymed | 2.5 |
| | | (#08-1110) | | 87-9663 | |

*IHC was performed using anti-PCNA (Invitrogen C/N 081110) primary antibody and developed with a prototype Next Generation SuperPicture ™ Detection Kit using multiple raw material sources of DAB.
#Staining intensity was scored following Invitrogen Quality Procedures, Document No. TM-041. Score range = 0.0-4.0

TABLE 30
Equivalency Testing of Next Generation Detection Reagents*

| | | | | Colon | | | Colon | Esophagus | | Basal | | Signal | |
|---|---|---|---|---|---|---|---|---|---|---|---|---|---|
| | | | Small | Adenocarcinoma | | | Mucinous | Carcinoma | | Cell | | Intensity | |
| DAB Source/Vendor | Uterus | Tonsil | Intestine | 1 | 2 | 3 | Carcinoma | 1 | 2 | Carc. | Glioma | Mean | SD |
| Invitrogen SuperPicture ™ | 3.0 | 3.5 | 3.25 | 3.0 | 3.75 | 3.0 | 3.75 | 2.5 | 3.5 | 2.75 | 3.0 | 3.18 | 0.40 |
| Next Generation Detection Reagents | 3.5 | 3.75 | 3.75 | 3.25 | 3.5 | 3.5 | 4.0 | 2.75 | 3.75 | 3.0 | 3.75 | 3.50 | 0.37 |
| Next Generation Detection Reagents | 3.25 | 3.5 | 4.0 | 3.5 | 3.75 | 3.75 | 3.75 | 2.5 | 3.5 | 3.0 | 3.75 | 3.48 | 0.43 |
| (30 days, 37° C.) | | | | | | | | | | | | | |
| ThermoPierce | 2.5 | 3.0 | 3.0 | 2.75 | 3.5 | 2.75 | 3.5 | 2.0 | 3.25 | 2.25 | 3.5 | 2.91 | 0.52 |
| Vector ImmPact ™ | 3.5 | 3.75 | 3.75 | 3.75 | 3.5 | 3.75 | 4.0 | 3.0 | 4.0 | 3.5 | 4.0 | 3.68 | 0.30 |
| Powervision Plus ™ | 3.0 | 3.5 | 3.5 | 3.5 | 3.5 | 3.75 | 4.0 | 2.75 | 4.0 | 3.25 | 3.75 | 3.50 | 0.39 |
| Biocare Cardassian w/ Enhancer | 2.75 | 3.0 | 3.25 | 3.25 | 3.25 | 3.25 | 3.75 | 2.5 | 3.5 | 3.0 | 3.5 | 3.18 | 0.36 |

*IHC was performed using Invitrogen's “ready to use” predilute 2^{nd} Generation primary antibodies targeting Proliferating Cell Nuclear Antigen (mouse anti-PCNA; #08-1110), Epidermal Growth Factor Receptor (mouse anti-EGFR; #08-1205), and Carcinoembryonic Antigen (mouse anti-CEA; #08-1057).
Human uterus, tonsil, and small intestine tissue samples (were assayed for PCNA expression.
Colon carcinoma tissue samples were assayed for CEA expression.
Esophagus carcinoma, basal cell carcinoma, and glioma tissue samples were assayed for EGFR expression.

1. A composition for stabilizing a chromogenic electron donor, said composition comprising:
a) a chelating agent;
b) a polyol; and
c) an antioxidant.

2. The composition of claim 1, wherein the chromogenic electron donor is diaminobenzidine (DAB).

3. The composition of claim 1, wherein the chelating agent is selected from the group consisting of DTPA, EDTA, EGTA, 1,10 phenanthroline, and diethylenetriaminepentamethylenephosphonic acid.

4. The composition of claim 3, wherein the chelating agent is DTPA.

5. The composition of claim 1, wherein the polyol is selected from the group consisting of propylene glycol, polyethylene glycol, and a sugar.

6. The composition of claim 5, wherein the polyol is propylene glycol.

7. The composition of claim 1, wherein the antioxidant is selected from the group consisting of sodium sulfite and sodium metabisulfite.

8. The composition of claim 7, wherein the antioxidant is sodium sulfite.

9. A composition, said composition comprising:
a) a chromogenic electron donor;
b) a chelating agent;
c) a polyol; and
c) an antioxidant,
wherein said chromogenic electron donor is stabilized by means of said chelating agent, said polyol, and said antioxidant.

10. The composition of claim 9, wherein the chromogenic electron donor is diaminobenzidine (DAB).

11. The composition of claim 9, wherein the chelating agent is selected from the group consisting of DTPA, EDTA, EGTA, 1,10 phenanthroline, and diethylenetriaminepentamethylenephosphonic acid.

12. The composition of claim 11, wherein the chelating agent is DTPA.

13. The composition of claim 9, wherein the polyol is selected from the group consisting of propylene glycol, polyethylene glycol, and a sugar.

14. The composition of claim 13, wherein the polyol is propylene glycol.

15. The composition of claim 9, wherein the antioxidant is selected from the group consisting of sodium sulfite and sodium metabisulfite.

16. The composition of claim 15, wherein the antioxidant is sodium sulfite.

17. A composition for stabilizing hydrogen peroxide, said composition comprising:
a) a buffer;
b) a chelating agent; and
c) a nitrogen-containing compound.

18. The composition of claim 17, wherein the buffer is an acetate buffer.

19. The composition of claim 17, wherein the chelating agent is DTPA.

20. The composition of claim 17, wherein the nitrogen-containing compound is imidazole.

21. A composition, said composition comprising:
a) hydrogen peroxide;
b) a buffer;
c) a chelating agent; and
d) a nitrogen-containing compound,
wherein said hydrogen peroxide is stabilized by means of said buffer, said chelating agent, and said nitrogen-containing compound.

22. The composition of claim 21, wherein the buffer is an acetate buffer.

23. The composition of claim 21, wherein the chelating agent is DTPA.

24. The composition of claim 21, wherein the nitrogen-containing compound is imidazole.

25-37. (canceled)
